(19) United States
(12) Patent Application Publication
(10) Pub. No.: US 2019/0017029 A1
(43) Pub. Date: Jan. 17, 2019

(54) Generation of Expandable Cardiovascular Progenitor Cells

(71) Applicant: The J. David Gladstone Institutes, a testament trust established under the Will of J. David, San Francisco, CA (US)

(72) Inventors: Yu Zhang, Day City, CA (US); Nan Cao, Guangzhou (CN); Sheng Ding, Orinda, CA (US)

(21) Appl. No.: 16/076,824

(22) PCT Filed: Feb. 24, 2017
(86) PCT No.: PCT/US2017/019295

§ 371 Date: Aug. 9, 2018

Related U.S. Application Data
(60) Provisional application No. 62/299,583, filed on Feb. 25, 2016.

(51) Int. Cl. C12N 5/077; A61K 35/34; A61P 9/00; C12N 5/071

(52) U.S. Cl. CPC C12N 5/0657; C12N 2501/2303; A61K 35/34; A61P 9/00; C12N 5/069; C12N 2500/90; C12N 2506/03; C12N 2501/2306; C12N 2501/115; C12N 2501/2311; C12N 2501/125; C12N 2501/135; C12N 2501/33; C12N 2501/15; C12N 2506/02; C12N 2501/155; C12N 2501/16; C12N 2501/165; C12N 5/0661

(57) ABSTRACT

Methods and compositions for expanding cardiovascular progenitor cells are described herein that include use of compositions and culture media that have at least the following components: BMP4, Activin A, a glycogen synthase kinase 3 inhibitor, and an inhibitor of FGF, VEGF, and PDGF signaling. The methods include contacting cardiovascular progenitor cells with a culture medium having BMP4, Activin A, a glycogen synthase kinase 3 inhibitor, and an inhibitor of FGF, VEGF, and PDGF signaling, to generate an expanded cardiovascular progenitor cell population.

### PRIORITY APPLICATIONS

This application claims the benefit of priority to U.S. Provisional Application Ser. No. 62/299,583, filed Feb. 25, 2016, the contents of which are specifically incorporated herein by reference in their entity.

### BACKGROUND

Heart failure (HF) is a devastating disease and major cause of morbidity and mortality worldwide. Heart failure often follows myocardial infarction (MI) that is usually accompanied by a massive loss of cardiomyocytes (CMs). These CMs cannot be regenerated by the adult mammalian heart and cannot yet be replaced and/or regenerated via cell-based therapies. Unfortunately, transplanting CMs into an infarcted heart yields only transient and marginal benefits (Burridge et al., 2012). Shortly after transplantation, most CMs are soon lost. These effects are likely caused by the limited proliferative capacity of fully differentiated CMs as well as deficient blood-vessel formation to supply oxygen and nutrients (Lam et al., 2009). Thus, to create more effective regenerative therapies, a cell type is needed that can be extensively expanded in vitro and that can robustly differentiate into cardiovascular cells in a diseased heart.

### SUMMARY

Methods and compositions are described herein for generating cells with extensive proliferative ability and restricted cardiovascular differentiation potential. These cells can be generated from pluripotent stem cells or by conversion of adult cell types into cardiovascular progenitor cells using a conversion medium. The cells so generated are referred to herein as induced, expandable cardiovascular progenitor cells (ieCPCs). The ieCPCs can be propagated robustly in the optimized chemically defined conditions that include BMP4, Activin A, a glycogen synthase kinase 3 inhibitor, and an inhibitor of FGF, VEGF, and PDGF signaling. The ieCPCs can be propagated and sub-cultured (passaged) for more than 18 passages. For example, more than 10¹⁶ ieCPCs can be generated from 10⁵ starting fibroblasts. Even when expanded long-term, the ieCPCs broadly express cardiac-signature genes and retain their potential for single-step, direct differentiation into functional cardiomyocytes (CMs), endothelial cells (ECs), and smooth muscle cells (SMCs) in vitro. When transplanted into infarcted mouse hearts, ieCPCs spontaneously generated CMs, ECs, and SMCs and improved heart performance for up to 12 weeks post-infarction. Therefore, ieCPCs can be employed for powerful new cardiac-regenerative therapies.

One aspect of the invention is method for expanding cardiovascular progenitor cells comprising contacting the cardiovascular progenitor cells with a culture medium that includes BMP4, Activin A, a glycogen synthase kinase 3 inhibitor, and an inhibitor of FGF, VEGF, and PDGF signaling.

### DESCRIPTION OF THE FIGURES

FIG. 1A-1M illustrate the generation and characterization of ieCPCs. FIG. 1A is a schematic diagram of hypothesis-driven screening protocol for identifying conditions that can expand ieCPCs, where D means day, DOX means doxycycline; and JI1 means Jak inhibitor 1. FIG. 1B illustrates expression of cardiovascular progenitor cell (CPC) markers on day 14 as detected by qPCR of cells cultured in basal medium (open bars) or of cells cultured in medium containing BACS (BMP, Activin A, CHIR99021, and SU5402)(speckled bars). As shown essentially no cardiovascular progenitor cell (CPC) markers are expressed by cells cultured in basal medium. FIG. 1C shows representative flow-cytometry analyses illustrating the percentage of Flk-1⁺/PdgfR-α⁺ (F⁺/P⁺) cells treated with BACS (BMP, Activin A, CHIR99021, and SU5402). Basal ieCPC medium without BACS served as the control. FIG. 1D graphically illustrates the percentage of Flk-1⁺/PdgfR-α⁺ (F⁺/P⁺) cells treated with BACS (BMP, Activin A, CHIR99021, and SU5402) over time (days 8-14). Basal ieCPC medium without BACS served as the control. FIG. 1E shows representative flow-cytometry analyses for detection of cTnT-expressing cells that may be differentiated from freshly isolated Flk-1⁻/PdgfR-α⁻ (F⁻/P⁻), Flk-1⁻/PdgfR-α⁺ (F⁻/P⁺), and Flk-1⁺/PdgfR-α⁺ (F⁺/P⁺) cells treated with either basal differentiation medium, BMP4, or IWP2. FIG. 1F graphically illustrates the percentage of cTnT⁺ cells as detected by flow cytometry in the cell population differentiated from freshly isolated Flk-1⁻/PdgfR-α⁻ (F⁻/P⁻), Flk-1⁻/PdgfR-α⁺ (F⁻/P⁺), and Flk-1⁺/PdgfR-α⁺ (F⁺/P⁺) cells treated with either basal differentiation medium or IWP2 (left graph); or the percentage of cTnT cells as detected by flow cytometry in the cell population differentiated from freshly isolated Flk-1⁺/PdgfR-α⁺ (F⁺/P⁺) cells treated with basal differentiation medium, BMP4, or IWP2. FIG. 1G graphically illustrates the percentage of cTnT⁺ in cells differentiated from Flk-1⁺/PdgfR-α⁺ (F⁺/P⁺) cells at passage 2 (P2) treated with IWP2 as detected by flow-cytometry. FIG. 1H shows expression of cardiomyocyte (CM) markers cTnI and cTnT, endothelial cell (EC) markers CD31 and VE-cadherin, and smooth muscle cell (SMC) markers α-SMA and calponin as detected by immunofluorescence in ieCPCs cultured in basal differentiation medium for 10 days. FIG. 11I shows expression of Gata4, Mef2c, Isl1, Nkx2-5, Gata4, and Ki-67 in purified Flk-1⁺/PdgfR-α⁺ (F⁺/P⁺) cells as detected by immunofluorescence. Scale bars, 100 μm. Data are means±S.E., n≥3. *P<0.05; **P<0.01; n.s., P>0.05. FIG. 1J graphically illustrates cTnT expression in cells differentiated from F⁻/P⁻, F⁻/P⁺, and F⁺/P⁺ cells as detected by immunostaining (staining intensity multiplied by area). **P<0.01. n.s., P>0.05. FIG. 1K graphically illustrates cTnT expression as detected by immunostaining of Flk-1⁺/PdgfR-α⁺ (F⁺/P⁺) cells treated with basal differentiation medium (basal), 20 ng/ml BMP4, or 5 μM IWP2. **P<0.01. FIG. 1L graphically illustrates relative expression of mesodermal-related genes during the generation of ieCPCs as detected by quantitative PCR (qPCR). All gene expression levels were normalized to expression levels of secondary mouse embryonic fibroblasts (2^{nd} MEFs) on day −2. FIG. 1M graphically illustrates relative expression of cardiac progenitor cell-related genes during the generation of ieCPCs by quantitative PCR (qPCR). All gene expression levels were normalized to the expression levels of 2^{nd} MEFs on day −2.

FIG. 2A-2K illustrate some of the characteristic of isolated ieCPCs expanded long term under chemically defined conditions. FIG. 2A graphically illustrates growth of ieCPCs during long-term expansion with BACS (BMP, Activin A. CHIR99021, and SU5402). FIG. 2B shows representative images illustrating the typical morphology of ieCPCs at passage 3, 8, and 18. FIG. 2C shows the percentage of Flk-1⁺/PdgfR-α⁺ (F⁺/P⁺) cells as detected by flow cytometry at passage 3, 10, and 18. FIG. 2D shows expression of Gata4, Mef2c, Ki-67, Nkx2-5, and Isl1 in ieCPCs at passage 15 as detected by immunofluorescence. Scale bars, 100 μm. FIG. 2E shows the percentage of F⁺/P⁺ cells detected by flow cytometry after culturing with BACS or after removing individual components. FIG. 2F graphically illustrates the percentage of F⁺/P⁺ cells as detected by flow cytometry after culturing with BACS or with individual components of BACS omitted. FIG. 2G graphically illustrates the cell number after culturing with BACS or removing individual components. Data were collected after three passages (n=3). The symbol—indicates that the compound was omitted; the letter B means BMP4; the letter A means Activin A; the letter C means CHIR99021; the letter S means SU5402. Data are means±S.E., *P<0.05; **P<0.01. FIG. 2H graphically illustrates Nanog, Esrrb, and Zfp42 expression in cells treated with 2 μg/ml doxycycline (DOX) from day 0 to 6 and in cells cultured in ieCPC- or iPSC-reprogramming conditions from day 7 to 14 as detected by qPCR analysis. FIG. 2I graphically illustrates the relative expression of the cardiomyocyte genes Tnrt2 (left graph) and Myl2 (right graph) as examined by qPCR when cells were cultured in BASCS or in media without one of the BACS compounds (−B means BMP4 removed; −A means Activin A removed; —C means CHIR99021 removed; —S means SU5402 removed). FIG. 2J graphically illustrates the relative expression of the endothelial cell (EC) genes Pecam1 (left graph) and Cdh5 (right graph) as examined by qPCR of ieCPCs cultured in BACS, or in BACS without one of the BACS compounds from BACS components (−B means BMP4 removed; −A means Activin A removed; −C means CHIR99021 removed; —S means SU5402 removed). FIG. 2K graphically illustrates the relative expression of smooth muscle cell (SMC) genes Tag/n (left graph) and Cnn1 (right graph) in ieCPCs cultured in BACS or after removal of individual compounds from BACS, as examined by qPCR. The symbol—means the medium was depleted of the indicated compound; the letter B means BMP4; the letter A means Activin A; the letter C means CHIR99021; and the letter S means SU5402. Scale bars, 100 μm. Data are means±S.E., *P<0.05.

FIG. 3A-3F illustrates that ieCPCs acquire the transcriptional signatures of developing CPCs. FIG. 3A illustrates transcriptome analysis revealing differences in gene expression among passage 3 (P3) and 12 (P12) ieCPCs, as well as their parental MEFs, cells at reprogramming D9 (D9), and ieCPC cardiac derivatives (ieCPC-CDs) as detected by RNA-seq. FIG. 3B graphically illustrates gene ontology (GO) analyses of upregulated and downregulated genes in ieCPCs P3/MEFs. FIG. 3C shows a summary of principal component analysis of the global gene-expression profile across all tested cell types, where the circle symbols show data for cells generated as described herein, the square symbols represent data by Devine et al. (eLife 3 (2014); D_): and the triangle symbols represent data by Wamstad et al. (Cell 151: 206-220 (2012);_W). The terms Pos and Tot represent data for CPCs with or without purification with a Smarcd3-GFP⁺ reporter, respectively. The abbreviation CPs means cardiac progenitors; the abbreviation MES means mesoderm. FIG. 3D-1 to 3D-3 illustrate expression of cardiovascular progenitor cell (CPC)-related marker genes in all tested samples as detected by RNA-seq. FIG. 3D-1 illustrates expression of CPC-related transcription factors marker genes. FIG. 3D-2 illustrates expression of CPC-related chromatin remodeler marker genes. FIG. 3D-3 illustrates expression of CPC-related cell-signaling molecules. FIG. 3E graphically summarizes gene-ontology (GO) analyses of up-regulated (top) and down-regulated (bottom) genes in ieCPCs passage 3 (P3) cells at reprogramming day 9 (D9). FIG. 3F graphically summarizes GO analyses of up-regulated (top) and down-regulated (bottom) genes in ieCPCs P3/ieCPC cardiac derivatives (ieCPC-CDs).

FIG. 4A-4I illustrates that ieCPCs expanded long term efficiently differentiate into functional cardiomyocytes in vitro. FIG. 4A illustrates expression of multiple cardiomyocyte (CM) markers in ieCPC-CMs as detected by immunofluorescence analyses. Scale bars, 100 μm. FIG. 4B shows a heat map illustrating expression of cardiomyocyte transcripts in MEFs, ieCPCs, and ieCPC cardiac derivatives (ieCPC-CDs; identified as CDs), and primary neonatal ventricle (Neo ventricle). FIG. 4C illustrates the percent of ieCPC-CMs expressing cTnT after 10 days of differentiation by late passage (P10-15) ieCPCs, as detected by flow-cytometry analyses. FIG. 4D illustrates expression of α-actinin and cTnT in ieCPC-CMs as detected by immunofluorescence analyses. The right panels show the boxed areas that were taken from the left panels and so that the right panels are at higher magnification. Scale bars, 20 μm. FIG. 4E shows images of ieCPC-CMs obtained by transmission electron microscopy, where the arrows identify Z-bands; the area or brackets between two arrows identify sarcomeric units; and the asterisks identify mitochondria. Scale bar, 1 μm. FIG. 4F shows representative traces of simultaneous action potentials (APs) (identified as changes in membrane potential (Eₘ)) and Ca²⁺ transients (Fluo-4 fluorescence expressed relative to baseline (F/F₀)) in ieCPC-CMs. FIG. 4G shows tabulated parameters describing action potentials (APs): maximum upstroke velocity (dV/dtₘₐₓ); overshoot potential (OSP); minimum diastolic potential (MDP); AP duration at 500/% and 90% of repolarization (APD₅₀ & APD₉₀); and Ca²⁺ transients: peak relative fluorescence (Peak CaT) and Ca²⁺-transient duration from 10% of the rising phase to 90% decay (CaTD_{10-90%}.). FIG. 4H shows the effects of isoproterenol and carbachol on beating frequency in ieCPC-CMs (*P<0.05, n=6), where the left graph illustrates beating frequency when isoproterenol or carbachol are added, and the right graph shows the mean beating frequency when isoproterenol or carbachol is present. FIG. 4I illustrates caffeine-induced release of Ca²⁺ from sarcoplasmic reticulum in ieCPC-CMs, where the left trace shows the Ca²⁺ transients, and the right graph illustrates the peak height of the CPC-CM Ca²⁺ transients (**P<0.01, n=10). Data are the mean values ±S.E.

FIG. 5A-5G shows that ieCPCs expanded long term efficiently differentiate into functional ECs and SMCs in vitro. FIG. 5A illustrates expression of endothelial cell (EC) markers in ieCPC-ECs as detected by immunofluorescence analyses. FIG. 5B illustrates the percent of cells expressing CD31 after 10 days of maintaining late stage ieCPCs under EC differentiation conditions, as detected by flow-cytometry analyses. FIG. 5C shows that ieCPC-ECs, but not control 2^{nd} MEFs, form a capillary-like network on a thin layer of matrigel. FIG. 5D shows uptake of ac-LDL by ieCPC-ECs (bottom images), but not by control 2^{nd} MEFs (top images). FIG. 5E shows expression of smooth muscle cell (SMC) markers in ieCPC-SMCs as detected by immunofluorescence. FIG. 5F shows that ieCPC-SMCs, but not control ieCPCs, display similar contractile ability compared to primary SMCs in response to 100 μM carbachol. FIG. 5G graphically illustrates the percent cell-surface area contraction of each cell type, summarized from 29 ieCPC-SMCs, 28 primary SMCs, and 28 ieCPCs. Data are means±S.E., **P<0.01. Scale bars, 100 μm.

FIGS. 6A-6L illustrate that ieCPCs give rise to CMs, ECs, and SMCs in vivo and improve cardiac function after myocardial infarction (MI). FIG. 6A shows expression of RFP and cardiomyocyte (CM) markers in tissue sections collected 2 weeks after transplanting passage 10 RFP-labeled ieCPCs into infarcted hearts of immunodeficient mice, as detected by immunofluorescence analyses. FIGS. 6B and 6C show expression of RFP and endothelial cell (EC) markers in tissue sections collected 2 weeks after transplanting passage 10 RFP-labeled ieCPCs into infarcted hearts of immunodeficient mice, as detected by immunofluorescence analyses. FIGS. 6D and 6E show expression of RFP and SMC markers in tissue sections collected 2 weeks after transplanting passage 10 RFP-labeled ieCPCs into infarcted hearts of immunodeficient mice, as detected by immunofluorescence analyses. Scale bars, 100 μm. FIGS. 6F and 6G graphically illustrate the ejection fraction (left graphs) and fractional shortening (right graphs) of the left ventricle (LV) quantified by echocardiography. FIG. 6F shows results from a first independent experiment. FIG. 6G shows results from a second independent experiment. D, days; W, weeks. FIG. 6H schematically illustrates the location of sections taken of representative hearts that are shown in FIG. 6I-6K. FIG. 6I illustrates masson-trichrome stained heart sections taken at eight levels (L1-L8) for detection of cardiac fibrosis 12 weeks after coronary ligation and administration of 2^{nd} MEFs or ieCPCs. The ligation site is marked as X. Sections of representative hearts are shown in (FIG. 6I) with quantification in (FIG. 6J). Scar tissue (%)=(the sum of fibrotic area or length at L1-L8/the sum of left ventricle area or circumference at L1-L8)×100. Scale bars, 500 μm. FIG. 6J graphically illustrates the percent scar tissue area measured in two of the heart sections shown in FIGS. 6H and 6I. FIG. 6K graphically illustrates the left ventricle (LV) circumference of mouse hearts 12 weeks after transplantation of 2^{nd} MEFs or ieCPCs. Data was summarized from 48 sections for each group. Data are means±S.E., *P<0.05. FIG. 6L shows representative images illustrating teratoma formation in infarcted mouse heart injected with mouse embryonic stem cells (mESCs) (left panel) and lack of tumor formation in heart injected with ieCPCs (right panel). Scale bars, 1 mm.

FIG. 7A-7F illustrate that BACS captures and expands CPCs derived from mESCs. FIG. 7A shows representative images illustrating the typical morphology of mESC-derived CPCs cultured in BACS at passage 5 and 10. FIG. 7B illustrates the percentage of Flk-1⁺/PdgfR-α⁺ (F⁺/P⁺) cells detected by flow cytometry at passages 5 and 10. FIG. 7C illustrates expression of Ki-67, Nkx2-5, Gata4, Mef2c, and Isl1 in mESC-derived CPCs at passage 10 as detected by immunofluorescence. FIG. 7D illustrates expression of CM, EC, and SMC markers in mESC-derived CPCs cultured in CM-, EC-, and SMC-specific differentiation conditions for 10 days as detected by immunofluorescence. Scale bars, 100 μm. FIG. 7E illustrates percentage of cells expressing cTnT, CD31, and α-SMA in mESC-derived CPCs cultured in the same differentiation conditions as in FIG. 7D, as detected by flow-cytometry analyses. FIG. 7F illustrates hierarchical clustering analysis of the cell types indicated along the x-axis based on expression of pluripotent, mesodermal, CPC-, and CM-specific markers detected by qPCR.

FIG. 8A-8E illustrates reprogramming of Tail-Tip fibroblasts into ieCPCs. FIG. 8A shows representative images illustrating the typical morphology of ieCPCs derived from tail-tip fibroblasts (TTF-ieCPCs) at passages 2 and 10. FIG. 8B illustrates the percentage of Flk-1+/PdgfR-α+ cells detected by flow cytometry at passages 2 and 10 of TTF-ieCPCs. FIG. 8C shows representative images illustrating expression of Gata4, Mef2c, Ki-67, Nkx2-5, and Isl1 in TTF-ieCPCs at passage 10, as detected by immunofluorescence. FIG. 8D shows representative images illustrating expression of cardiomyocyte (CM; cardiac) markers cTnT and cTnI, endothelial cell (EC) markers CD31 and VE-cadherin, and smooth muscle cell (SMC) markers α-SMA and calponin as detected by immunofluorescence in TTF-ieCPCs cultured in CM-, EC-, and SMC-specific differentiation conditions for 10 days. Scale bars, 100 μm. FIG. 8E illustrates the percent of cells expressing the cardiomyocyte marker cTnT, the endothelial cell marker CD31, and the SMC marker α-SMA in TTF-ieCPCs cultured in the same differentiation conditions as in FIG. 8D.

### DETAILED DESCRIPTION

Methods and compositions are described herein for expanding cardiovascular progenitor cells that involve contacting the cardiovascular progenitor cells with a culture medium comprising BMP4, Activin A, a glycogen synthase kinase 3 inhibitor, and an inhibitor of FGF, VEGF, and PDGF signaling.

### Expansion of Cardiovascular Progenitor Cells

Cardiovascular progenitor cells (CPCs) that can be expanded include those that express Gata4, Mef2c, Tbx5, and Nkx2-5, Flk-1, Pdg/R-α, or any combination thereof. In some instances the cardiovascular progenitor cells express at least the following two markers: FIk-1 and Pdg/R-α (i.e., the cells are Flk-1⁺/PdgfR-α⁺ cells). The cardiovascular progenitor cells express such markers before, during, and after expansion.

Expansion of the cardiovascular progenitor cells is conducted in vitro in a culture medium that includes BMP4, Activin A, a glycogen synthase kinase 3 inhibitor, and an inhibitor of FGF, VEGF, and PDGF signaling. The combination of BMP4, Activin A, a glycogen synthase kinase 3 inhibitor, and an inhibitor of FGF, VEGF, and PDGF signaling is referred to herein as “BACS.” In some instances, Jak inhibitor 1 and/or ascorbic acid can be present in the culture medium with the BACS components.

Bone morphogenetic protein-4 (BMP-4) is a member of the group of bone morphogenic proteins and a ventral mesoderm inducer. BMP-4 can be included in a culture medium for expansion of cardiovascular progenitor cells at a concentration of about 0.5 to 50 ng/mL, about 1.0-30 ng/mL, about 1.5-20 ng/mL, about 2.0-15 ng/mL, about 2.5-10 ng/mL, about 3-8 ng/mL, about 4-6 ng/mL, or any range derivable therein. In certain cases, BMP-4 is included in the culture media at a concentration of about 1, 2, 3, 4, 5, 6, 7, 8, 9, or about 10 ng/mL. As shown in the Examples provided herein, cardiovascular progenitor cells expand well in a concentration of BMP-4 that is about 5 ng/mL. A concentration of about 5 ng/mL BMP-4 can therefore be employed in the culture media for expanding cardiovascular progenitor cells.

Activin A is a member of the TGF-β family first identified in late 1980s as an inducer of follicle-stimulating hormone. Activin A is highly conserved in evolution and throughout the animal kingdom. It regulates a variety of biologic processes including cell proliferation, hematopoiesis, wound healing, fibrosis, and mesodermal development. Activin A signals through the Activin type I (Alk 4 or 7) and type II (ActRII or ActRIIB) receptors and shares with TGFβ the activation of the Smad cascade. See, Phillips et al., Cytokine Growth Factor Rev. 20(2): 153-64 (2009); Werner, Cytokine Growth Factor Rev. 17(3): 157-71 (2006).

Activin A can be included in a culture medium at a concentration, for example, from about 0.5 ng/ml to about 100 ng/ml, or from about 1.0 ng/ml to about 75 ng/ml, or from about 2 ng/ml to about 50 ng/ml, or from about 3 ng/ml to about 40 ng/ml, or from about 5 ng/ml to about 30 ng/ml, or from about 6 ng/ml to about 20 ng/ml, or from about 7 ng/ml to about 15 ng/ml, or from about 8 ng/ml to about 12 ng/ml, or about 10 ng/ml.

Examples of glycogen synthase kinase 3 (GSK3) inhibitors that can be employed include one or more of the following compounds:
CHIR99021 (6-(2-(4-(2,4-dichlorophenyl)-5-(4-methyl-1H-imidazol-2-yl)pyrimidin-2-ylamino)ethylamino)nicotinonitrile);
1-azakenpaullone (9-Bromo-7,12-dihydro-pyrido[3′,2′:2,3]azepino[4,5-b]indol-6(5H)-one), BIO ((2′Z,3′E)-6-Bromoindirubin-3′-oxime);
AR-A014418 (N-(4-Methoxybenzyl)-N′-(5-nitro-1,3-thiazol-2-yl)urea);
Indirubin-3′-monoxime;
5-Iodo-indirubin-3′-monoxime;
kenpaullone (9-Bromo-7,12-dihydroindolo-[3,2-d] [1]benzazepin-6(5H)-one);
SB-415286 (3-[(3-Chloro-4-hydroxyphenyl)amino]-4-(2-nitro-phenyl)-1H-pyrrole-2,5-dione);
SB-216763 (3-(2,4-Dichlorophenyl)-4-(1-methyl-1H-indol-3-yl)-1H-pyrrole-2,5-dione);
Maybridge SEW00923SC (2-anilino-5-phenyl-1,3,4-oxadiazole);
(Z)-5-(2,3-Memylenedioxyphenyl)imidazolidine-2,4-dione,
TWS119 (3-(6-(3-aminophenyl)-7H-pyrrolo[2,3-d]pyrimidin-4-yloxy)phenol);
CHIR98014 (N2-(2-(4-(2,4-dichlorophenyl)-5-(1H-imidazol-1-yl)pyrimidin-2-ylamino)ethyl)-5-nitropyridine-2,6-diamine);
SB415286 (3-(3-chloro-4-hydroxyphenylamino)-4-(2-nitrophenyl)-1H-pyrrole-2,5-dione);
Tideglusib (also known as NP031112, or NP-12; 1,2,4-Thiadiazolidine-3,5-dione, 2-(1-naphthalenyl)-4-(phenylmethyl));
LY2090314 (1H-Pyrrole-2,5-dione, 3-imidazo[1,2-a]pyridin-3-yl-4-[1,2,3,4-tetrahydro-2-(1-piperidinylcarbonyl)pyrrolo[3,2,1-jk][1,4]benzodiazepin-7-yl]);
lithium salt (e.g., LiCl); or
any combination thereof.

For example, the glycogen synthase kinase 3 (GSK3) inhibitor can, for example, be CHIR99021, SB216763, TWS119, CHIR98014, Tideglusib, SB415286, LY2090314, or any combination thereof. In some embodiments, the glycogen synthase kinase 3 (GSK3) inhibitor can be CHIR99021, whose structure is shown below.

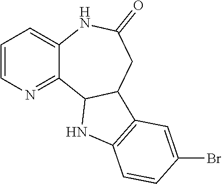 [SMILES: O=C1CC2c3cc(Br)ccc3NC2c2ncccc2N1]

The glycogen synthase kinase 3 (GSK3) inhibitors can also be in the form of a salt or hydrate of any of the foregoing compounds. Methods and assays for determining a level of GSK-3 inhibition are available to a skilled person and include, for example, the methods and assays described in Liao et al., Endocrinology, 145(6): 2941-2949 (2004); and in U.S. Pat. No. 8,323,919, both of which are specifically incorporated by reference herein in their entireties.

To increase the proportion of cells that express markers indicative of a cardiovascular progenitor phenotype, a selected population of cells is contacted or mixed with one or more GSK3 inhibitors for a time and at a concentration sufficient to differentiate or re-direct the cells to a cardiovascular progenitor lineage.

Glycogen synthase kinase 3 (GSK3) inhibitors can be employed in the compositions and methods described herein in a variety of amounts and/or concentrations. For example, one or more glycogen synthase kinase 3 (GSK3) inhibitors can be employed at a concentration of about 0.01 micromolar to about 1 millimolar in a solution, or about 0.1 micromolar to about 100 micromolar in a solution, or about 0.5 micromolar to about 10 micromolar in a solution, or about 1 micromolar to about 5 micromolar in a solution. In some cases one or more glycogen synthase kinase 3 (GSK3) inhibitors can be employed at a concentration of about 1 micromolar.

The inhibitor of FGF, VEGF, and PDGF signaling can be any of the following:
SU 5402 (chemical name 2-[(1,2-Dihydro-2-oxo-3H-indol-3-ylidene)methyl]-4-methyl-1H-pyrrole-3-propanoic acid), available from Tocris (see website at tocris.com/dispprod.php?ItemId=217279#. VsYARVKxVps;
PD173074 (chemical name N-[2-[[4-(Diethylamino)butyl]amino]-6-(3,5-dimethoxyphenyl)pyrido[2,3-d]pyrimidin-7-yl]-N′-(1,1-dimethylethyl)urea) available from Tocris (see website at tocris.com/dispprod.php?ItemId=190969#.Vs4HM1KxVps);
AP 24534 (also known as ponatinib; chemical name 3-(2-Imidazo[1,2-b]pyridazin-3-ylethynyl)-4-methyl-N-[4-[(4-methyl-1-piperazinyl)methyl]-3-(trifluoromethyl)phenyl]-benzamide) available from Tocris (see website at tocris.com/dispprod.php?ItemId=298351 #.VsX8wlKxVpshttps;
FIIN 1 hydrochloride (chemical name N-(3-((3-(2,6-dichloro-3,5-dimethoxyphenyl)-7-(4-(diethylamino)butylamino)-2-oxo-3,4-dihydropyrimido[4,5-d]pyrimidin-1(2H)-yl)methyl)phenyl)acrylamide), available from Tocris (see website at tocris.com/dispprod.php? ItemId=271572#.VsX-DVKxVps;
R 1530 (chemical name 5-(2-Chlorophenyl)-7-fluoro-1,2-dihydro-8-methoxy-3-methylpyrazolo[3,4-b][1,4]benzodiazepine), available from Tocris (see website at tocris.com/dispprod.php?ItemId=376348#. VsX_P1KxVps;
SU 6668 (chemical name 5-[1,2-Dihydro-2-oxo-3H-indol-3-ylidene)methyl]-2,4-dimethyl-1H-pyrrole-3-propanoic acid), available from Tocris (see website at tocris.com/dispprod.php?ItemId=219395#. VsYApFKxVps;
Sunitinib malate (also known as SU 11248; chemical name N-[2-(Diethylamino)ethyl]-5-[(Z)-(5-fluoro-1,2-dihydro-2-oxo-3H-indol-3-ylidine)methyl]-2,4-dimethyl-1H-pyrrole-3-carboxamide (2S)-2-hydroxybutanedioate salt), available from Tocris (see website at tocris.com/dispprod.php?ItemId=243827#. VsYBFFKxVps;
Toceranib (also known as Palladia; chemical name 5-[(Z)-(5-Fluoro-1,2-dihydro-2-oxo-3H-indol-3-ylidene)methyl]-2,4-dimethyl-N-[2-(1-pyrrolidinyl)ethyl]-1H-pyrrole-3-carboxamide), available from Tocris (see website at tocris.com/dispprod.php?ItemId=253448#. VsYBplKxVps; and/or
Brivanib alaninate (also known as BMS-582664; chemical name (S)-(R)-1-((4-((4-fluoro-2-methyl-1H-indol-5-yl)oxy)-5-methylpyrrolo[2,1-f][1,2,4]triazin-6-yl)oxy)propan-2-yl 2-aminopropanoate) available from Bristol-Myers Squibb.

The inhibitor of FGF, VEGF, and PDGF signaling can be employed in the compositions and methods described herein in a variety of amounts and/or concentrations. For example, one or more FGF, VEGF, and PDGF signaling inhibitors can be employed at a concentration of about 0.01 micromolar to about 1 millimolar in a solution, or about 0.1 micromolar to about 100 micromolar in a solution, or about 0.5 micromolar to about 10 micromolar in a solution, or about 1 micromolar to about 5 micromolar in a solution. In some cases one or more FGF, VEGF, and PDGF signaling inhibitors can be employed at a concentration of about 2 micromolar.

The number of cardiovascular progenitor cells seeded into culture media containing the BACS factors for expansion can vary. For example, cells can be seeded into BACS culture media at a density of about 1×10³ to about 1×10⁸ cells/ml, or a density of about 1×10⁴ to about 1×10⁷ cells/ml, or a density of about 1×10⁵ to about 1×10⁶ cells/ml. If only small numbers of cardiovascular progenitor cells are available, the cell population can be pre-expanded on a feeder layer of cells, or in a small volume. For example, small numbers of cardiovascular progenitor cells can be pre-expanded in microtiter wells at a density of about 1×10² to about 1×10⁴ cells/well, where the BACS and other useful factors can be present in the cell culture medium.

The time of contacting or mixing the BACS with cardiovascular progenitor cells can vary. For example, the cardiovascular progenitor cells can be cultured in a medium containing BACS for at least 2 days, at least 5 days, at least 10 days, at least 15 days, at least 20 days, at least 23 days, at least 25 days, at least 30 days, at least 50 days, at least 100 days, at least 200 days, at least 250 days, at least 500 days.

The cardiovascular progenitor cells can be subcultured or passaged while being expanded or just maintained in a culture medium containing BACS. For example, the cardiovascular progenitor cells can be passaged or subcultured at least about 10 times, or at least about 15 times, or at least about 16 times, or at least about 18 times, or at least about 20 times, or at least about 25 times, or at least about 50 times, or at least about 100 times. In some cases, the cardiovascular progenitor cells can be passaged or subcultured for about 1 to about 200 passages (subculturings), or for about 2 to about 100 passages (subculturings), or for about 5 to about 50 passages (subculturings), or for about 10 to about 30 passages (subculturings), or for about 15 to about 25 passages (subculturings).

In some instances the cardiovascular progenitor cells can be grown up (expanded) by at least 5-fold, or at least 10-fold, or at least 20-fold, or at least 50 fold, or at least 100-fold, or at least 1000-fold, or at least 10,000-fold, or at least 100,000-fold, or at least 1,000,000-fold, or at least 10,000,000-fold, or at least 100,000,000-fold, or at least 1,000,000,000-fold, or at least 10,000,000,000-fold, or at least 100,000,000,000-fold.

A Jak inhibitor 1 (JI1) can be present for at least one day in the medium before, during, or after the cardiovascular progenitor cells are being expanded. The Jak inhibitor 1 (JI1) can therefore be present in the culture medium with the BACS. The Jak inhibitor 1 (JI1) is also known as P6, Pyridone 6, DBI, JAK1 Inhibitor I, JAK2 Inhibitor I, JAK3 Inhibitor X. The chemical name for the Jak inhibitor 1 is 2-(1,1-Dimethylethyl)-9-fluoro-3,6-dihydro-7H-benz[h]-imidaz[4,5-f]isoquinolin-7-one); and it is available from EMD Millipore (see website at emdmillipore.com/US/en/product/InSolution % E2%84% A2-JAK-Inhibitor-I---CAS-457081-03-7--Calbiochem,EMD_BIO-420097.

For example, the Jak inhibitor 1 can be employed at a concentration of about 0.01 micromolar to about 500 micromolar in a solution, or about 0.05 micromolar to about 100 micromolar in a solution, or about 0.1 micromolar to about 10 micromolar in a solution, or about 0.2 micromolar to about 5 micromolar, or about 0.3 micromolar to about 1 micromolar in a solution. In some cases the Jak inhibitor 1 can be employed at a concentration of about 0.5 micromolar.

Ascorbic acid can also be present in the culture medium employed for expansion of cardiovascular progenitor cells. For example, ascorbic acid can be employed at a concentration of about 1 micromolar to about 1000 micromolar in a solution, or about 10 micromolar to about 700 micromolar in a solution, or about 50 micromolar to about 500 micromolar in a solution, or about 100 micromolar to about 400 micromolar, or about 150 micromolar to about 350 micromolar in a solution. In some cases ascorbic acid can be present in the expansion medium at a concentration of about 250 micromolar.

The base media employed to which the BACS, Jak inhibitor 1, and/or ascorbic acid factors are added can be a convenient cell culture medium.

The term “cell culture medium” (also referred to herein as a “culture medium” or “medium” or“culture media”) as referred to herein is a medium for culturing cells containing nutrients that maintain cell viability and support proliferation. The cell culture medium can contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are available to those skilled in the art.

Examples cell culture media that can be employed include mTESR-1 medium (StemCell Technologies, Inc., Vancouver, Calif.), or Essential 8′ medium (Life Technologies, Inc.) on a Matrigel substrate (BD Biosciences, NJ) or on a Corning® Synthemax surface, or in Johansson and Wiles CDM supplemented with insulin, transferrin, lipids and polyvinyl alcohol (PVA) as substitute for Bovine Serum Albumin (BSA). Examples of commercially available media also include, but are not limited to, Dulbecco's Modified Eagle's Medium (DMEM), Minimal Essential Medium (MEM), Basal Medium Eagle (BME), knockout DMEM, Advanced DMEM/FI2, RPM1 1640, Ham's F-10, Ham's F-12, a-Minimal Essential Medium (aMEM), Glasgow's Minimal Essential Medium (G-MEM), Iscove's Modified Dulbecco's Medium, or a general purpose media modified for use with pluripotent cells, such as X-VIVO (Lonza) or a hematopoietic base media.

The culture media can contain a variety of supplements such as serum, knockout serum replacement (KSR), embryonic stem cell (ESC)-qualified FBS, Glutamax, non-essential amino acids, β-mercaptoethanol (β-ME), nucleosides, nucleotides, ESC-qualified nucleosides, N2 supplement, B27 (with or without Vitamin A), Glutamax, bovine serum albumin (BSA), and combinations thereof.

### Obtaining Cardiovascular Progenitor Cells (CPCs)

The cardiovascular progenitor cells to be expanded can be obtained from a variety of sources. For example, the cardiovascular progenitor cells can be generated from adult or embryonic cells as described herein or as described in WO 2015/038704 (specifically incorporated herein by reference in its entirety). In another example, the cardiovascular progenitor cells can be generated by differentiation of stem cells into cardiovascular progenitor cells. The stem cells can be pluripotent stem cells, induced pluripotent stem cells, multipotent stem cells, unipotent stem cells, or combinations thereof.

For example, a starting population of cells can be converted into cardiovascular progenitor cells. Such a starting population of cells can be derived from essentially any source, and can be heterogeneous or homogeneous. In certain embodiments, the cells to be converted into cardiovascular progenitor cells are adult cells, including essentially any accessible adult cell type(s). In other embodiments, the cells used according to the invention are adult stem cells, progenitor cells, or somatic cells. In still other embodiments, the cells treated with any of the compositions and/or methods described herein include any type of cell from a newborn, including, but not limited to newborn cord blood, newborn stem cells, progenitor cells, and tissue-derived cells (e.g., somatic cells). The starting population can include essentially any live somatic cell type or stem cell. Such a starting population of cells can be reprogrammed, for example, by the compositions and/or methods described in WO 2015/038704.

As illustrated herein, fibroblasts can be reprogrammed to cross lineage boundaries and to be directly converted to a cardiovascular progenitor cell type.

Various cell types from all three germ layers have been shown to be suitable for somatic cell reprogramming by genetic manipulation, including, but not limited to liver and stomach (Aoi et al., Science 321(5889):699-702 (2008); pancreatic β cells (Stadtfeld et al., Cell Stem Cell 2: 230-40 (2008); mature B lymphocytes (Hanna et al., Cell 133: 250-264 (2008); human dermal fibroblasts (Takahashi et al., Cell 131, 861-72 (2007); Yu et al., Science 318(5854) (2007); Lowry et al., Proc Natl Acad Sci USA 105, 2883-2888 (2008); Aasen et al., Nat Biotechnol 26(11): 1276-84 (2008); meningiocytes (Qin et al., J Biol Chem 283(48):33730-5 (2008); neural stem cells (DiSteffano et al., Stem Cells Devel. 18(5): (2009); and neural progenitor cells (Eminli et al., Stem Cells 26(10): 2467-74 (2008). Any such cells can be reprogrammed and/or programmed to generate cardiovascular progenitor cells.

In some cases, the cells can be autologous or allogeneic cells (relative to a subject to be treated or who may receive the cells). Thus, somatic cells or adult stem cells can be obtained from a mammal suspected of having or developing a cardiac condition or a cardiac disease, and the cells so obtained can be converted (reprogrammed) into cardiovascular progenitor cells that are expanded using the compositions and methods described herein.

### Reprogramming Methods

Starting cells can be a source of cardiovascular progenitor cells that are expanded using the compositions and methods described herein. In particular, starting cells from a variety of sources can be converted or reprogrammed into cardiovascular progenitor cells. Starting cells are treated for a time and under conditions sufficient to convert the starting cells across lineage and/or differentiation boundaries to form cardiovascular progenitor cells. The methods described herein, and/or the methods described in WO 2015/038704 can be employed for such conversion.

As described herein, expression of transgenes encoding reprogramming factors such as Oct4, Sox2, KIf4, c-Myc, or combinations thereof can be employed to convert differentiated starting cells to a less differentiated state. For example, starting cells can be infected with a lentivirus harboring a doxycyline-inducible transgene encoding one or more of such reprogramming factors as described by Carey et al. (Proc Natl Acad Sci USA 106: 157-162 (2009)) or the expression of a single factor (Oct4) as described in Wang et al. (Cell Rep 6: 951-960 (2014)) can be used to convert differentiated starting cells to a less differentiated state.

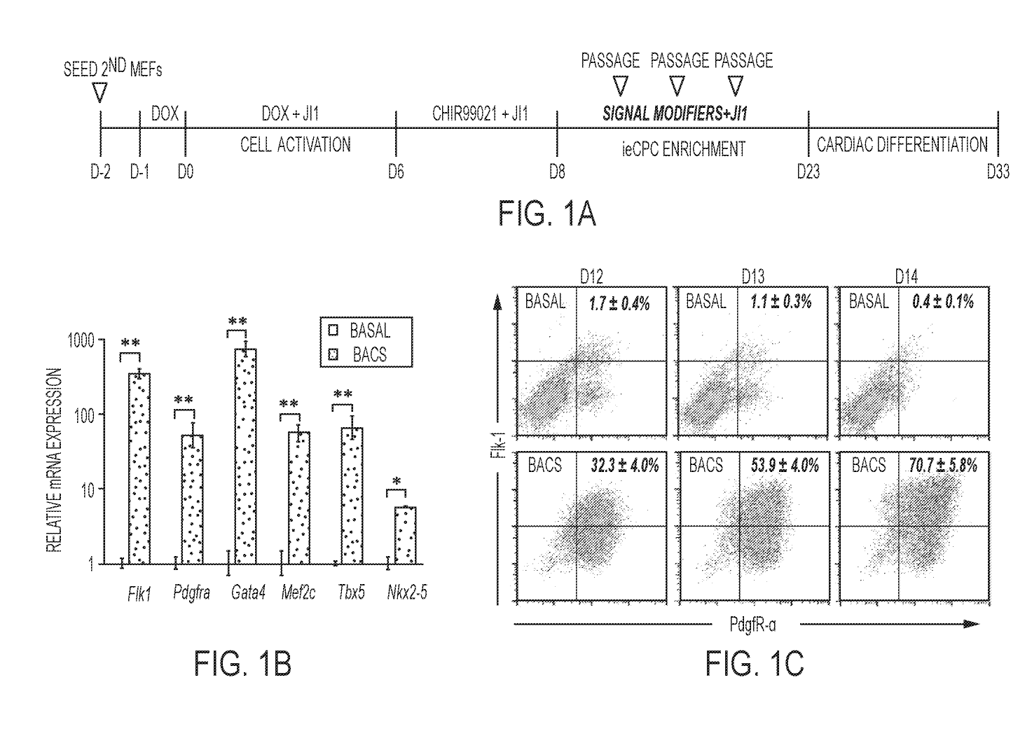

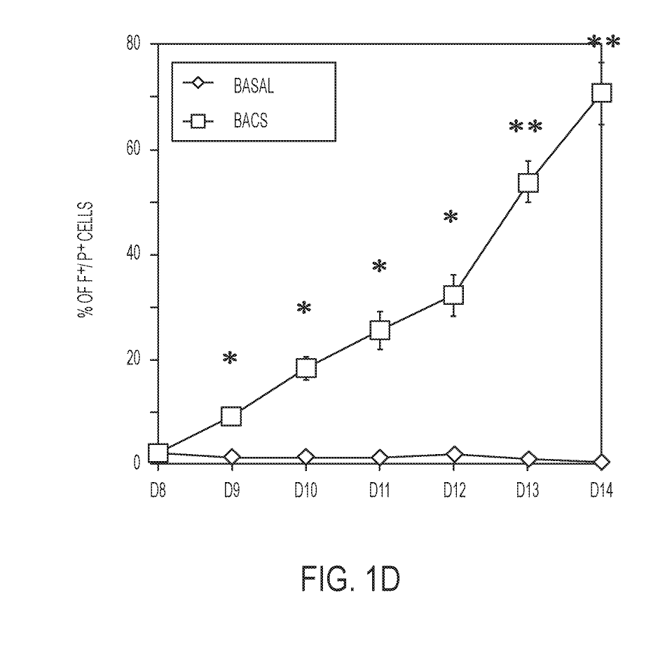

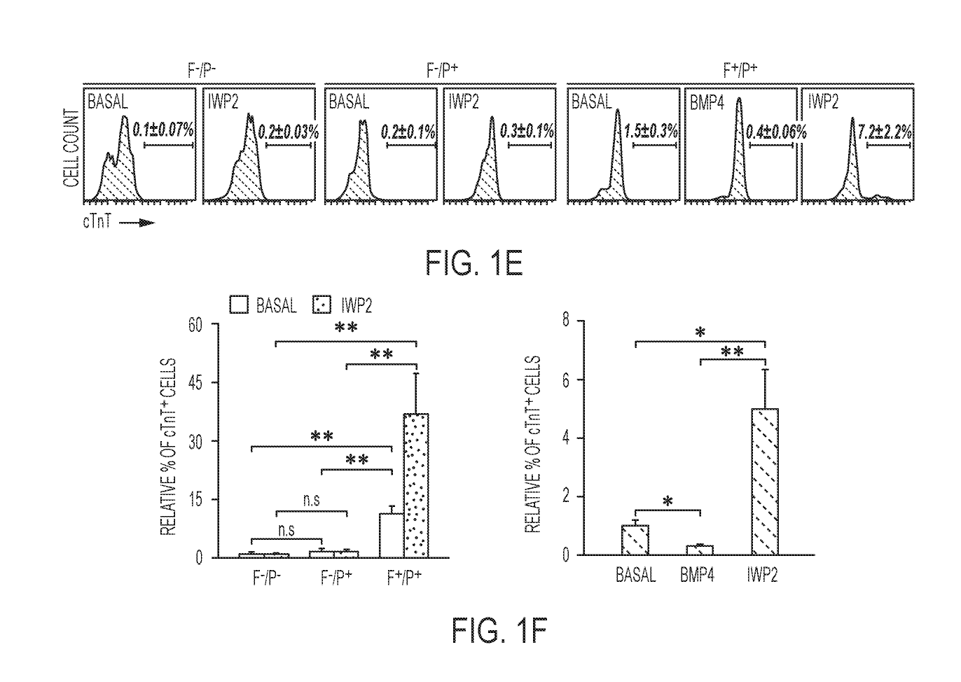

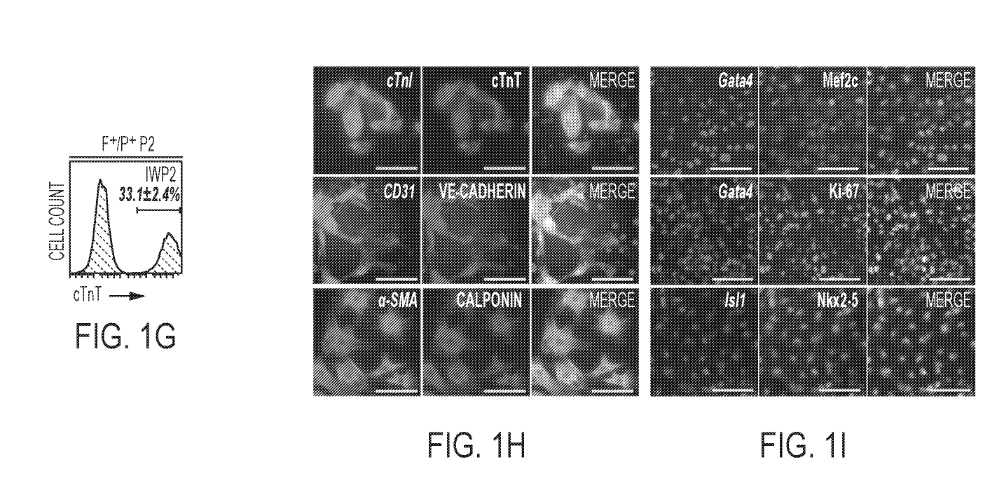

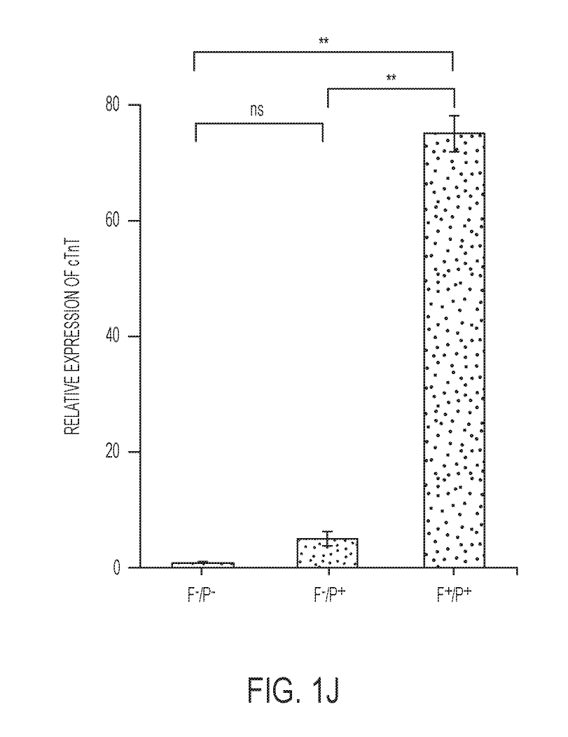

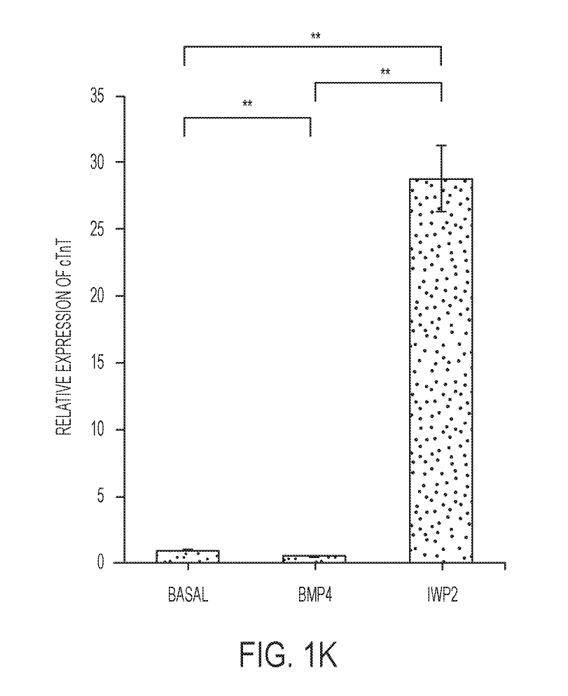

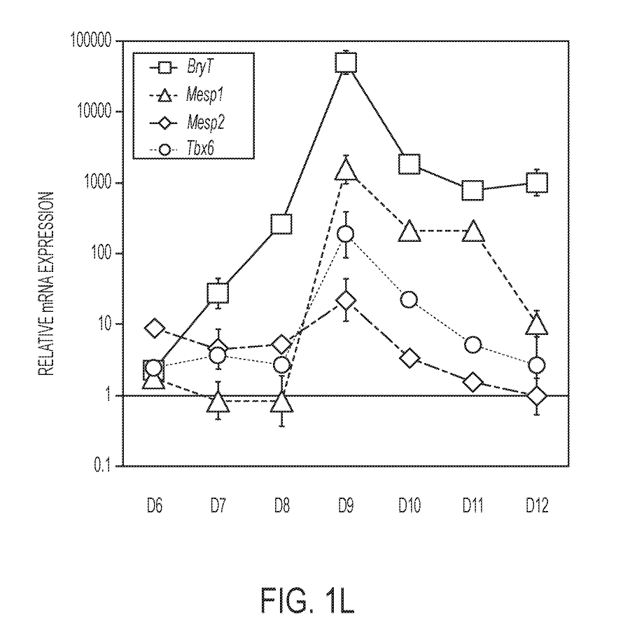

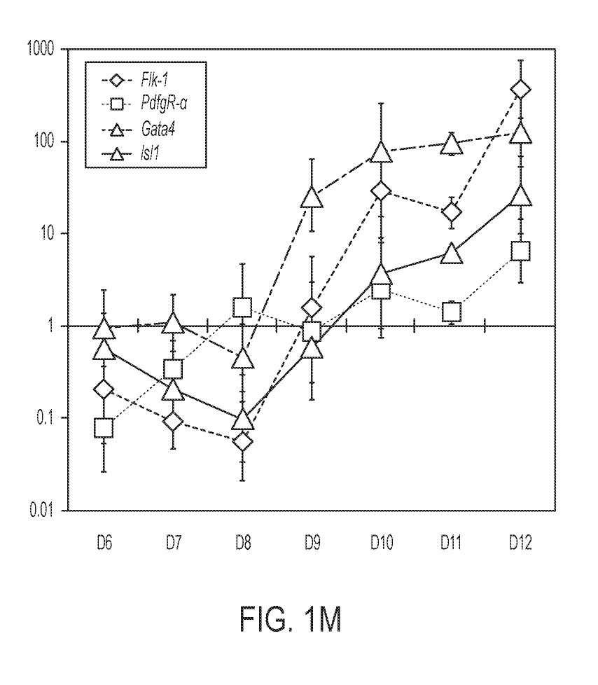

Stem cells and/or cells converted into a less differentiated state can be differentiated or reprogrammed into the cardiac lineage to generate cardiovascular progenitor cells by a variety of methods. For example, after treatment to convert somatic cells to a less differentiated state, the cells can be cultured in a reprogramming medium that includes knockout DMEM supplemented with 5% knockout serum replacement (KSR), 15% embryonic stem cell (ESC)-qualified FBS, 1× Glutamax, 1× non-essential amino acids, 0.1 mM β-mercaptoethanol (β-ME), 1% ESC-qualified nucleosides (Millipore), and 0.5 μM Jak inhibitor 1 (JI1; Millipore). About 2 μg/ml doxycycline can be present in the media when doxycycline is used to induce expression of one or more reprogramming factors such as Oct4, Sox2, Klf4, and c-Myc. The day when the cells are first contacted with the reprogramming medium is deemed to be day 0 in the figures provided herewith (see, e.g., FIG. 1A). The reprogramming medium can be renewed every 1-4 days, or at about every 2 days.

After about six days in the reprogramming medium, the medium can be changed to transdifferentiation medium. The transdifferentiation medium can include knockout DMEM supplemented with 14% knockout serum replacement (KSR), 1% ESC-qualified FBS, 1× Glutamax, 1× nonessential amino acids (NEAA), 0.1 mM β-mercaptoethanol (β-ME), 1% ESC-qualified nucleosides, a GSK-3 inhibitor (e.g., 3 μM CHIR99021; Stemgent), and 0.5 μM Jak inhibitor 1 (JI1; Millipore)).

After about two days in the transdifferentiation medium (i.e. at about day 8), the medium can be switched to ieCPC basal medium. The ieCPC basal medium can include Advanced DMEM/F12: Neural basal (1:1) supplemented with 1×N2, 1×B27 without Vitamin A, IX Glutamax, IX NEAA, 0.05% bovine serum albumin (BSA), and 0.1 mM β-ME.

The BACS factors (BMP4, Activin A, one or more glycogen synthase kinase 3 (GSK3) inhibitors, and one or more FGF, VEGF, and PDGF signaling inhibitors) can be added to the transdifferentiation medium at this point (day 8). Jak inhibitor 1 (JI1) can also be included in the transdifferentiation medium, for example, to suppress the establishment of pluripotency until the cells are ready for processing or purification. In some cases, ascorbic acid and/or other supplements can be present in the transdifferentiation medium. The transdifferentiation medium can be renewed every 1 to 4 days, or every 2 days.

As described herein, the Flk-1⁺/PdgfR-α⁺ cardiovascular progenitor cells are most robustly and stably expanded in media containing the BACS factors. Moreover, the Flk-1⁺/PdgfR-α⁺ cardiovascular progenitor cells express cardiac-signature genes and retain their potential for single-step, direct differentiation into functional cardiomyocytes (CMs), endothelial cells (ECs), and smooth muscle cells (SMCs) in vitro. When transplanted into infarcted mouse hearts, the Flk-1⁺/PdgfR-α⁺ cardiovascular progenitor cells spontaneously generate CMs, ECs, and SMCs. In addition, the infarcted hearts of such mice exhibit improved heart performance for up to 12 weeks post-infarction. Therefore, the FIk-1⁺/PdgfR-α⁺ cardiovascular progenitor cells can be employed for powerful new cardiac-regenerative therapies.

Hence, in some cases Flk-1⁺/PdgfR-α⁺ CPCs can be purified either to enrich the population of cells that will be expanded or to provide a population of expanded cells. Such purification can be performed, for example, by one or more rounds of FACS sorting

Pursuant to the methods described in WO2015/038704, the types of starting cells described herein can be incubated with a reprogramming composition that contains one or more GSK3 inhibitors/WNT agonists, TGF-beta inhibitors, inhibitors of extracellular signal-regulated kinase 1 (ERK1), inhibitors of Ras GTPase-activating protein (Ras-GAP)), Oct-4 activators, p160ROCK inhibitors (where p160ROCK is a rho-associated protein kinase), activators of cardiac myosin, inhibitors of G9a histone methyltransferase, inhibitors of various growth factor receptors such as PDGF receptor beta, protein kinase receptor inhibitors, inhibitors of PDGF-BB receptor, and any combination thereof. The composition can contain at least three of the agents, or at least four of the agents, or at least five of the agents, or at least six of the agents, or at least seven of the agents, or at least eight of the agents.

For reprogramming, the starting cells can be dispersed in a cell culture medium that contains the reprogramming composition at a density that permits cell expansion. For example, about 1 to 10⁴ to about 1 to 10¹⁰ cells can be contacted with the reprogramming composition in a selected cell culture medium, especially when the cells are maintained at a cell density of about 1 to about 10⁸ cells per milliliter, or at a density of about 100 to about 10⁷ cells per milliliter, or at a density of about 1000 to about 10⁶ cells per milliliter.

The time for conversion of starting cells into cardiovascular progenitor cells can vary. For example, the starting cells can be incubated with the reprogramming composition until cardiovascular progenitor cell markers are expressed. Such cardiovascular progenitor cell markers can include any of the following markers: Flk-1, PdgfR-α, NKX2-5. MEF2c, GATA4, ISL1, and any combination thereof. As described herein, cardiovascular progenitor cells that express Flk-1 and PdgfR-α, two cell surface markers, are particularly suited for purification and expansion to generate useful populations of into cardiovascular progenitor cells.

Further incubation of the cells can be performed until expression of late stage cardiac progenitor markers such as Flk-1, PdgfR-α, NKX2-5, MEF2c, or a combination thereof occurs. The late stage cardiac progenitor markers such as Flk-1, PdgfR-α, NKX2-5 and/or MEF2c can be expressed by about 14 days, or by about 15 days, or by about 16 days, or by about 17, or by about 18 days of incubation of cells using the compositions and methods described herein.

In some instances, a reprogrammed population of cells (at a selected stage of reprogramming), or a population of cardiovascular progenitor cells (ieCPCs) can be frozen at liquid nitrogen temperatures, stored for periods of time, and then thawed for use at a later date. If frozen, a population cells can be stored in a 10% DMSO, 30% FCS, within 60% ieCPC basal medium. Once thawed, the cells can be expanded by culturing the cells in a medium that can contain the BACS factors, as well as other selected factors such as growth factors, vitamins, feeder cells, and other components selected by a person of skill in the art.

### Therapy

Cardiovascular progenitor cells are a promising avenue for cardiac-regenerative therapy. These cells evolve from the mesoderm during cardiogenesis, which is a well-orchestrated process in developing embryos that can be recapitulated in differentiating pluripotent stem cells (PSCs). Patterned mesoderm gives rise to a hierarchy of downstream cellular intermediates that represent lineage-restricted CPCs of fully differentiated heart cells, including CMs, endothelial cells (ECs), and smooth muscle cells (SMCs) (Burridge et al., 2012). Each step in this hierarchy is tightly controlled by multiple stage-specific signals (e.g., Wnt, Activin/Nodal, bone morphogenetic protein (BMP), fibroblast growth factor (FGF), and Notch) (Burridge et al., 2012; Bruneau, 2013). Additionally, the gradual loss of multipotency, or commitment of cell fate, is usually accompanied by a decreased capacity of cellular proliferation. Thus, by isolating CPCs that can extensively self-renew and possess multiple, but restricted, potentials to directly differentiate into these three cardiovascular-cell types, we may encourage the development of more effective and potentially safer therapies for cardiac regeneration.

The expanded populations of cardiovascular progenitor cells (ieCPCs) generated as described herein can be employed for tissue reconstitution or regeneration in a mammal such as human patient, a domesticated animal, a zoo animal, or other mammalian subject. The mammal can be in need of such treatment. The ieCPCs broadly express cardiac-signature genes and retain their potential for single-step, direct differentiation into functional cardiomyocytes (CMs), endothelial cells (ECs), and smooth muscle cells (SMCs) both in vitro and in vivo.

For therapy, cardiovascular progenitor cells (ieCPCs), or cells generated therefrom can be administered locally or systemically. Such cells are administered in a manner that permits them to graft or migrate to a diseased or injured tissue site and to reconstitute or regenerate the functionally deficient area. Devices are available that can be adapted for administering cells, for example, to cardiac tissues.

A population of ieCPCs can be introduced by injection, catheter, implantable device, or the like. A population of ieCPCs can be administered in any physiologically acceptable excipient or carrier that does not adversely affect the cells. For example, the ieCPCs can be administered intravenously or through an intracardiac route (e.g., epicardially or intramyocardially). Methods of administering the ieCPCs to subjects, particularly human subjects, include injection or implantation of the cells into target sites in the subjects. The cells of the invention can be inserted into a delivery device which facilitates introduction of the cells after injection or implantation of the device within subjects. Such delivery devices include tubes, e.g., catheters, for injecting cells and fluids into the body of a recipient subject. The tubes can additionally include a needle, e.g., a syringe, through which the cells of the invention can be introduced into the subject at a desired location. The kits described herein can include such devices.

The ieCPCs and/or cells derived therefrom can be inserted into such a delivery device, e.g., a syringe, in different forms. A population of cells can be supplied in the form of a pharmaceutical composition. Such a composition can include an isotonic excipient prepared under sufficiently sterile conditions for human administration. For general principles in medicinal formulation, the reader is referred to CELL THERAPY: STEM CELL TRANSPLANTATION, GENE THERAPY, AND CELLULAR IMMUNOTHERAPY, by G. Morstyn & W. Shendan eds, Cambridge University Press, 1996; and HEMATOPOIETIC STEM CELL THERAPY, E. D. Ball, J. Lister & P. Law, Churchill Livingstone, 2000. The choice of the cellular excipient and any accompanying constituents of the composition that includes a population of cells can be adapted to optimize administration by the route and/or device employed.

As used herein, the term “solution” includes a carrier or diluent in which the cells of the invention remain viable. Carriers and diluents which can be used with this aspect of the invention include saline, aqueous buffer solutions, physiologically acceptable solvents, and/or dispersion media. The use of such carriers and diluents is well known in the art. The solution is preferably sterile and fluid to allow syringability. For transplantation, a solution containing a suspension of cells can be drawn up into a syringe, and the solution containing the cells can be administrated to anesthetized transplantation recipients. Multiple injections may be made using this procedure.

The cells can also be embedded in a support matrix. A composition that includes a population of cells can also include or be accompanied by one or more other ingredients that facilitate engraftment or functional mobilization of the reprogrammed cells. Suitable ingredients include matrix proteins that support or promote adhesion of the reprogrammed cells, or complementary cell types, such as cardiac pacemaker cells, or cardiac cells at different stages of maturation. In another embodiment, the composition may include physiologically acceptable matrix scaffolds. Such physiologically acceptable matrix scaffolds can be resorbable and/or biodegradable.

The population of cells generated by the methods described herein can include low percentages of non-cardiac cells (e.g., fibroblasts). For example, a population of reprogrammed cells for use in compositions and for administration to subjects can have less than about 90% non-cardiac cells, less than about 85% non-cardiac cells, less than about 80% non-cardiac cells, less than about 75% non-cardiac cells, less than about 70% non-cardiac cells, less than about 65% non-cardiac cells, less than about 60% non-cardiac cells, less than about 55% non-cardiac cells, less than about 50%/o non-cardiac cells, less than about 45% non-cardiac cells, less than about 40% non-cardiac cells, less than about 35% non-cardiac cells, less than about 30% non-cardiac cells, less than about 25% non-cardiac cells, less than about 20/o non-cardiac cells, less than about 15% non-cardiac cells, less than about 12% non-cardiac cells, less than about 10% non-cardiac cells, less than about 8% non-cardiac cells, less than about 6% non-cardiac cells, less than about 5% non-cardiac cells, less than about 4%0 non-cardiac cells, less than about 3% non-cardiac cells, less than about 2% non-cardiac cells, or less than about 1% non-cardiac cells of the total cells in the cell population.

In some embodiments, the therapeutic compositions containing ieCPCs and other useful ingredients are administered in a “therapeutically effective amount.” Such a therapeutically effective amount is an amount sufficient to obtain the desired physiological effect, e.g., treatment of a condition, disorder, disease and the like or reduction in symptoms of the condition, disorder, disease and the like. For example, the therapeutic agents can be administered to treat any of the conditions, disorders, or diseases described herein. Examples include congestive heart failure, myocardial infarction, cardiac ischemia, myocarditis, arrhythmia or any combination thereof.

Many cell types are capable of migrating to an appropriate site for regeneration and differentiation within a subject. To determine the suitability of various therapeutic administration regimens and dosages of cell compositions, the cells can first be tested in a suitable animal model. At one level, cells are assessed for their ability to survive and maintain their phenotype in vivo. Cells can also be assessed to ascertain whether they migrate to diseased or injured sites in vivo, or to determine an appropriate dosage such as an appropriate number of cells and/or a frequency of administration of cells. Cell compositions can be administered to immunodeficient animals (such as nude mice, or animals rendered immunodeficient chemically or by irradiation). Tissues can be harvested after a period of regrowth, and assessed as to whether the administered cells or progeny thereof are still present, are alive, and/or have migrated to desired or undesired locations.

Injected cells can be traced by a variety of methods. For example, cells containing or expressing a detectable label (such as green fluorescent protein, or beta-galactosidase) can readily be detected. The cells can be pre-labeled, for example, with BrdU or [³H]-thymidine, or by introduction of an expression cassette that can express green fluorescent protein, or beta-galactosidase. Alternatively, the reprogrammed cells can be detected by their expression of a cell marker that is not expressed by the animal employed for testing (for example, a human-specific antigen). The presence and phenotype of the administered population of reprogrammed cells can be assessed by fluorescence microscopy (e.g., for green fluorescent protein, or beta-galactosidase), by immunohistochemistry (e.g., using an antibody against a mouse or human antigen), by ELISA (using an antibody against a human antigen), or by RT-PCR analysis using primers and hybridization conditions that cause amplification to be specific for human polynucleotides.

A therapeutically effective dose of cells can be about 1×10⁴ to about 1×10⁹ cells, or about 1×10⁴ to about 1×10⁸ cells, or about 1×10⁵ to about 1×10⁸ cells/ml. The dose and the number of administrations can be optimized by those skilled in the art.

The therapeutic regimen can also administration of other active ingredients such as agents useful for treatment of cardiac diseases, conditions and injuries. Such other active ingredients can be administered separately or with the cells. Examples of other active ingredients that be administered include, for example, an anticoagulant (e.g., dalteparin (fragmin), danaparoid (orgaran), enoxaparin (lovenox), heparin, tinzaparin (innohep), and/or warfarin (coumadin)), an antiplatelet agent (e.g., aspirin, ticlopidine, clopidogrel, or dipyridamole), an angiotensin-converting enzyme inhibitor (e.g., Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), and/or Trandolapril (Mavik)), angiotensin II receptor blockers (e.g., Candesartan (Atacand), Eprosartan (Teveten), Irbesartan (Avapro), Losartan (Cozaar), Telmisartan (Micardis), and/or Valsartan (Diovan)), a beta blocker (e.g., Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), and/or Timolol (Blocadren)), Calcium Channel Blockers (e.g., Amlodipine (Norvasc, Lotrel), Bepridil (Vascor), Diltiazem (Cardizem, Tiazac), Felodipine (Plendil), Nifedipine (Adalat, Procardia), Nimodipine (Nimotop), Nisoldipine (Sular), Verapamil (Calan, Isoptin, Verelan), diuretics (e.g, Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol) and/or Spironolactone (Aldactone)), vasodilators (e.g., Isosorbide dinitrate (Isordil), Nesiritide (Natrecor), Hydralazine (Apresoline), Nitrates and/or Minoxidil), statins, nicotinic acid, gemfibrozil, clofibrate, Digoxin, Digitoxin, and/or Lanoxin.

Additional agents can also be included such as antibacterial agents, antimicrobial agents, anti-viral agents, biological response modifiers, growth factors; immune modulators, monoclonal antibodies and/or preservatives. The compositions of the invention may also be used in conjunction with other forms of therapy.

The Examples describe some of the experiments performed in the development of the invention and provide results illustrating that the ieCPCs survive and populate cardiac tissues in mammals suffering from myocardial infarction.

### Example 1: Materials and Methods

This Example describes some of the materials and methods employed in the development of the invention.

### Preparing Doxycyline-Inducible Secondary Mouse Embryonic Fibroblasts and Mouse Tail-Tip Fibroblasts

Secondary mouse embryonic fibroblasts (2^{nd} MEFs) harboring doxycyline (DOX)-inducible transgenes encoding Oct4, Sox2, Klf4, and c-Myc, and a Nanog-GFP reporter to monitor the establishment of pluripotency were derived using method described by Wernig et al. (Nat Biotechnol 26: 916-924 (2008)). Heads, spinal cords, and developing organs were carefully removed from embryos. 2^{nd} MEFs were cultured on gelatin-coated plates in MEF medium (high-glucose Dulbecco's Modified Eagle Medium (DMEM containing 10% fetal bovine serum (FBS) and IX nonessential amino acids (NEAA)).

Mouse tail-tip fibroblasts (TTFs) were prepared using methods described by Efe et al. (Nat Cell Biol 13: 215-222 (2011)) and Wang et al. (Cell Rep 6: 951-960 (2014)). Briefly, tail tips from neonatal and adult mice were minced with a sterile razor blade and then cultured in 10-cm culture dishes containing 2 ml MEF medium. After overnight culture, another 10 ml medium was added to the dish. Seven days later, fibroblasts that migrated out of the tissue samples were collected and expanded.

### Cell-Activation and Signaling-Directed System-Based Cardiac Reprogramming

A Cell-Activation and Signaling-Directed (CASD) system-based cardiac reprogramming was conducted using methods described by Efe et al. (2011). Briefly, 2^{nd} MEFs at passage 3 were seeded onto geltrex (Gibco)-coated plates at a density of 2.5×10⁴ cells/well of a 12-well plate in MEF medium (day −2). Doxycycline (DOX; 2 μg/ml, Sigma-Aldrich) was added into the medium one day later (day −1) and the cells were cultured for another day. Medium was then changed to reprogramming medium (knockout DMEM supplemented with 5% knockout serum replacement (KSR), 15% embryonic stem cell (ESC)-qualified FBS, 1× Glutamax, 1×NEAA, 0.1 mM β-mercaptoethanol (β-ME), 1% ESC-qualified nucleosides (Millipore), 0.5 μM Jak inhibitor 1 (JI1; Millipore), and 2 μg/ml DOX) at day 0. This medium was renewed every 2 days.

On day 6, the medium was changed to transdifferentiation medium (knockout DMEM supplemented with 14% KSR, 1% ESC-qualified FBS, 1× Glutamax, 1× nonessential amino acids (NEAA), 0.1 mM f-mercaptoethanol (β-ME), and 1% ESC-qualified nucleosides, 3 μM CHIR99021 (Stemgent), and 0.5 μM Jak inhibitor 1 (JI1; Millipore)).

On day 8, the medium was switched to ieCPC basal medium (Advanced DMEM/F12: Neural basal (1:1) supplemented with 1×N2, 1×B27 without Vitamin A, 1× Glutamax, 1×NEAA, 0.05% bovine serum albumin (BSA), and 0.1 mM β-ME) supplemented with BACS (5 ng/ml BMP4, 10 ng/ml Activin A, 3 μM CHIR99021, and 2 μM SU5402 (Tocris Bioscience)). JI1 (0.5 μM) was included to suppress the establishment of pluripotency until the cells were ready for purification at day 13. This medium was renewed every 2 days.

To reprogram genetically unmodified TTFs, cells were infected with lentivirus harboring a DOX-inducible transgene encoding the reprogramming factors (Carey et al., 2009) for 12 hours as previously described (Wang et al., 2014). They were cultured in MEF medium for 3 days to recover from viral infection and then seeded onto 12-well geltrex-coated plates at a density of 2.5×10⁴ cells/well in MEF medium and reprogrammed as 2^{nd} MEFs.

All cytokines are from a human source (R&D Systems), and all cultivation substances for cell cultures were from Life Technologies, unless stated otherwise.

Long-Term Expansion of ieCPCs

Flk-1⁺/PdgfR-α⁺ ieCPCs were purified on day 13 by fluorescence-activated cell sorting (FACS) and seeded onto 12-well geltrex-coated tissue culture plates at a density of 1×10⁶ cells/well in ieCPC basal medium with BACS. JI1 was removed, and 250 μM ascorbic acid (Sigma-Aldrich) was added to promote cell growth. Confluent ieCPCs were split by incubating with collagenase B (Roche) at 37° C. for 5 min, followed by accutase (Innovative Cell Technologies) at 37° C. for another 2-5 min. ieCPCs were routinely passaged every four days by seeding onto 12-well plates at a density of 5×10⁵ cells/well, and medium was renewed every two days. Rock inhibitors, such as Y27632 (10 μM, Tocris Bioscience) and thiazovivin (1 μM, Cellagen Technology), can improve ieCPCs survival during sorting, passaging, or recovering from cryopreservation, but were not absolutely necessary.

### Differentiation, Purification, and Expansion of CPCs Derived From Mouse Embryonic Stem Cells

CPC differentiation from mouse embryonic stem cells (mESCs) was performed as described by Kattman et al. (Cell Stem Cell 8, 228-240 (2011)). Briefly, mESCs were dissociated, aggregated in ultralow attachment plates (Corning) at 7.5×10⁴ cells/ml, and cultured for two days in serum-free differentiation (SFD) medium (IMDM: F12 (3:1) supplemented with 0.5×N2, 0.5×B27 without Vitamin A, 1× Glutamax, 0.05% BSA, 450 μM MTG (Sigma-Aldrich), and 250 μM ascorbic acid). After 48 hours, embryoid bodies (EBs) were dissociated and reaggregated for another 24 hours with 0.5 ng/ml BMP4, 5 ng/ml Activin A, 5 ng/ml VEGF, and 250 μM ascorbic acid.

Flk-1⁺/PdgfR-α⁺ CPCs were then purified by FACS, seeded onto 12-well geltrex-coated tissue culture plates at a density of 1×10⁶ cells/well in ieCPC basal medium with BACS and 250 μM ascorbic acid. The cell were routinely passaged every three days.

Differentiation of ieCPCs into Cardiovascular Lineages

For cardiac differentiation, ieCPCs were passaged onto 96-well matrigel-coated plates at a density of 3×10′ cells/cm² and cultured in serum-free differentiation (SFD) medium for 10 days. IWP2 (5 μM) was added to SFD medium during the first six days to increase the yield of cardiomyocytes (CMs) in the functional characterization assays.

For smooth muscle cell (SMC), endothelial cell (EC), and FBS-induced non-specific differentiation, ieCPCs were seeded onto matrigel-coated plates at a density of 1×10⁴ cells/cm², unless stated otherwise. For EC differentiation, ieCPCs were cultured in Endothelial Cell Growth Medium-2 (EGM™-2; Lonza) for 10 days. For SMC differentiation, ieCPCs were cultured in SFD medium supplemented with TGF-β1 (2 ng/ml) and PDGF-BB (10 ng/ml) for 10 days. For FBS-induced differentiation, ieCPCs were cultured in SFD medium supplemented with 10% FBS for 10 days.

Positive control cells were differentiated from mESCs following the protocol of Wobus et al. (Methods in Molecular Biology 185: 127-156 (2002)). Briefly, mESCs were cultivated as EBs for 2 days in DMEM supplemented with 15% FBS using standard hanging-drop methods. Formed EBs were then transferred into ultralow attachment plates and cultured in suspension for 4 days. At day 7, floating EBs were plated onto gelatin-coated dishes and cultured for another two weeks.

Differentiation efficiency of each cell type was calculated with qPCR, flow cytometry, and/or an In Cell Analyzer 2000 (General Electric Healthcare).

Long-Term Expansion of ieCPCs

Flk-1⁺/PdgfR-α⁺ cells were purified by FACS and seeded onto geltrex-coated plates in ieCPC basal medium supplemented with BACS and 250 μM ascorbic acid (Sigma). ieCPCs were routinely passaged every four days, and medium was renewed every two days.

Differentiation of ieCPCs and Mesoderm Precursors into Non-Cardiovascular Lineages

To induce hematopoietic differentiation, the second stage of hematopoietic specification of an established protocol by Grigoriadis et al. (Blood 115: 2769-2776 (2010)) was modified. Briefly, ieCPCs were seeded onto matrigel-coated plates at a density of 1×10⁴ cells/cm² and cultured in StemPro-34 medium containing VEGF (10 ng/ml), bFGF (1 ng/ml), IL-6 (10 ng/ml), IL-3 (40 ng/ml), IL-11 (5 ng/ml) and SCF (100 ng/ml) for 4 days.

For skeletal-muscle differentiation, the second stage of a protocol by Mizuno et al. (FASEB J. 24: 2245-2253 (2010)) was modified. Briefly, ieCPCs were aggregated in ultralow attachment plates at 2.5×10′ cells/ml in skeletal muscle-differentiation medium (DMEM supplemented with 5% horse serum, 1× Glutamax, 1×NEAA, and 0.1 mM β-ME) for 3 days of suspension culture. Cell aggregates were then dissociated, seeded onto matrigel-coated plates at a density of 1×10⁴ cells/cm², and cultured in skeletal-muscle-differentiation medium for 10 days.

Adipogenic and chondrogenic differentiation procedures were modifications of the methods described by Lee et al. (Cell Physiol Biochem 14: 311-324 (2004)). Briefly, for adipogenic differentiation, ieCPCs were seeded onto matrigel-coated plates at a density of 1×10⁴ cells/cm² and cultured in adipogenic-differentiation medium (α-MEM supplemented with 10% FBS, 1 μM dexamethasone (Sigma-Aldrich), 100 μg/ml 3-isobutyl-1-methylxanthine (Sigma-Aldrich), 5 μg/ml insulin, and 60 μM indomethacin (Sigma-Aldrich)) for 14 days. The presence of adipocytes was monitored by Oil Red-O (Sigma-Aldrich) staining. For chondrogenic differentiation, 10 μl concentrated ieCPCs (3×10⁶ cells/ml) were suspended in chondrogenic-differentiation medium (α-MEM supplemented with 1% FBS, 50 μg/ml ascorbic acid, 6.25 μg/ml insulin, and 10 ng/ml TGF-β1) were seeded into the center of each well of 24-well plates and allowed to attach at 37° C. for 2 hr. Chondrogenic-differentiation medium was then carefully added into the plates without detaching the cell aggregates. Cells were cultured in chondrogenic-differentiation medium for 2 weeks, dissociated, and replated onto matrigel-coated dishes for an additional week of culture before analysis. The presence of chondrocytes was monitored by Alizarin Red S (Sigma-Aldrich) staining.

For all non-cardiovascular-lineage differentiations, mesodermal precursors derived from E14 mESCs (harboring a Brachyury-GFP reporter that marks mesodermal precursors) were used as a positive control in parallel. Briefly, mESCs were cultivated as EBs for 2 days in DMEM supplemented with 20% FBS. Brachyury-GFP⁺ cells were sorted by FACS and treated in the same conditions as ieCPCs in all experiments involving non-cardiovascular-lineage differentiation.

### RT-PCR and Quantitative PCR

Total RNA was prepared using the RNeasy Plus Mini Kit with Qiashredder columns (Qiagen). On-column DNase digestion with RNase-free DNase (Qiagen) was performed to remove residual DNA. Total RNA (1 μg) was reverse transcribed into cDNA with the iScript cDNA Synthesis Kit (Bio-Rad). All quantitative PCR (qPCR) reactions were performed in triplicate with iQ SYBR Green Supermix (Bio-Rad) with one twentieth of a cDNA reaction per replicate, which was performed on an ABI 7900HT system (Invitrogen, Applied Biosystems). Expression data were analyzed with DataAssist V3.01 (Life Technologies). Each set of reactions was repeated with cDNA from at least three independent experiments. The primer sequences are listed in Table 1.

TABLE 1
Primer List for qPCR and RT-PCR

| Gene | Forward Primer sequence | Reverse Primer sequence |
|---|---|---|
| T | TTGAACTTTCCTCCATGTGCTGA | TCCCAAGAGCCTGCCACTTT |
| | (SEQ ID NO: 1) | (SEQ ID NO: 2) |
| Mesp1 | CATCGTTCCAGTACGCAGAA | CTAGAAGAGCCAGCATGTCG |
| | (SEQ ID NO: 3) | (SEQ ID NO: 4) |
| Mesp2 | CCCAGAGCCTAGGAACAAGA | GGGTTCTGGAGACACAGAAAG |
| | (SEQ ID NO: 5) | (SEQ ID NO: 6) |
| Tbx6 | CCGAGAAAATGGCAGAAACT | GTGTATCCCCACTCCCACAG |
| | (SEQ ID NO: 7) | (SEQ ID NO: 8) |
| Flk1 | AAACCTCCTGCAAGCAAATG | TCCAGAATCCTCTTCCATGC |
| | (SEQ ID NO: 9) | (SEQ ID NO: 10) |
| Pdgfra | AGAGGAGGAGCTTGAGGGAG | AGAAAATCCGATACCCGGAG |
| | (SEQ ID NO: 11) | (SEQ ID NO: 12) |
| Gata4 | CTGGAAGACACCCCAATCTC | CAGGCATTGCACAGGTAGTG |
| | (SEQ ID NO: 13) | (SEQ ID NO: 14) |
| Mef2c | TGGAGAGATGAAGTGAAGCG | GCACAGCTCAGTTCCCAAAT |
| | (SEQ ID NO: 15) | (SEQ ID NO: 16) |
| Tbx5 | GGCAGTGATGACCTGGAGTT | TGGTTGGAGGTGACTTTGTG |
| | (SEQ ID NO: 17) | (SEQ ID NO: 18) |
| Nkx2-5 | CCAAGTGCTCTCCTGCTTTC | GGCTTTGTCCAGCTCCACT |
| | (SEQ ID NO: 19) | (SEQ ID NO: 20) |
| Isl1 | TGTTTGAAATGTGCGGAGTG | GCATTTGATCCCGTACAACC |
| | (SEQ ID NO: 21) | (SEQ ID NO: 22) |
| Actc1 | AGCTGTCTTCCCGTCCATC | GCTCTGGGCTTCATCACCTA |
| | (SEQ ID NO: 23) | (SEQ ID NO: 24) |
| Kena5 | ATGAGGCCCATCACTGTAGG | AAAATTGGAGACGATGACGG |
| | (SEQ ID NO: 25) | (SEQ ID NO: 26) |
| Pln | CCCAGCTAAGCTCCCATAAG | AACAGGCAGCCAAATGTGA |
| | (SEQ ID NO: 27) | (SEQ ID NO: 28) |
| Atp2a2 | CTGGTGATATAGTGGAAATTGCTG | GGTCAGGGACAGGGTCAGTA |
| | (SEQ ID NO: 29) | (SEQ ID NO: 30) |
| Slc8a1 | TTGAGGACACCTGTGGAGTG | TTCTCATACTCCTCGTCATCG |
| | (SEQ ID NO: 31) | (SEQ ID NO: 32) |
| Kcnj2 | CCATGATCCTGTACCAGCAA | AGAGATGGATGCTTCCGAGA |
| | (SEQ ID NO: 33) | (SEQ ID NO: 34) |
| Cacna1c | CCTGCTGGTGGTTAGCGTG | TCTGCCTCCGTCTGTTTGGAA |
| | (SEQ ID NO: 99) | (SEQ ID NO: 100) |
| Scn5a | CTACCGCATAGTGGAGCACA | CGCTCCTCCAGGTAGATGTC |
| | (SEQ ID NO: 35) | (SEQ ID NO: 36) |
| Myh6 | GCGCATTGAGTTCAAGAAGA | CTTCATCCATGGCCAATTCT |
| | (SEQ ID NO: 37) | (SEQ ID NO: 38) |
| Myh7 | AAGGGCCTGAATGAGGAGTAG | TGCAAAGGCTCCAGGTCTGA |
| | (SEQ ID NO: 39) | (SEQ ID NO: 40) |
| Tnnt2 | GTGTGCAGTCCCTGTTCAGA | ACCCTCAGGCTCAGGTTCA |
| | (SEQ ID NO: 41) | (SEQ ID NO: 42) |
| Tnni3 | GCCTCTGGAGATCATCATGG | CTCGGTAGTTGGCAGAGGAG |
| | (SEQ ID NO: 43) | (SEQ ID NO: 44) |
| Myl2 | CTTCACTATCATGGACCAGAACAG | ACACTTTGAATGCGTTGAGAATGGT |
| | (SEQ ID NO: 45) | (SEQ ID NO: 46) |
| Myl7 | CTCACACTCTTCGGGGAGAA | CTCTTCCTTGTTCACCACCC |
| | (SEQ ID NO: 47) | (SEQ ID NO: 48) |
| Gja5 | CAGTTGAACAGCAGCCAGAG | AGCTCCAGTCACCCATCTTG |
| | (SEQ ID NO: 49) | (SEQ ID NO: 50) |
| Ryr2 | ACATCATGTTTTACCGCCTGAG | TTTGTGGTTATTGAACTCTGGCT |
| | (SEQ ID NO: 51) | (SEQ ID NO: 52) |
| Nanog | CCTCCAGCAGATGCAAGAACTC | CTTCAACCACTGGTTTTTCTGCC |
| | (SEQ ID NO: 53) | (SEQ ID NO: 54) |
| Zfp42 | TATGACTCACTTCCAGGGGG | AGAAGAAAGCAGGATCGCCT |
| | (SEQ ID NO: 55) | (SEQ ID NO: 56) |
| Esrrb | CTCGCCAACTCAGATTCGAT | AGAAGTGTTGCACGGCTTTG |
| | (SEQ ID NO: 57) | (SEQ ID NO: 58) |
| Pecam1 | ACGCTGGTGCTCTATGCAAG | TCAGTTGCTGCCCATTCATCA |
| | (SEQ ID NO: 59) | (SEQ ID NO: 60) |
| Cdh5 | CACTGCTTTGGGAGCCTTC | GGGGCAGCGATTCATTTTTCT |
| | (SEQ ID NO: 61) | (SEQ ID NO: 62) |
| Tek | GAGTCAGCTTGCTCCTTTATGG | AGACACAAGAGGTAGGGAATTGA |
| | (SEQ ID NO: 63) | (SEQ ID NO: 64) |
| Tagln | CAACAAGGGTCCATCCTACGG | ATCTGGGCGGCCTACATCA |
| | (SEQ ID NO: 65) | (SEQ ID NO: 66) |
| Myh11 | AAGCTGCGGCTAGAGGTCA | CCCTCCCTTTGATGGCTGAG |
| | (SEQ ID NO: 67) | (SEQ ID NO: 68) |
| Cnn1 | TCTGCACATTTTAACCGAGGTC | GCCAGCTTGTTCTTTACTTCAGC |
| | (SEQ ID NO: 69) | (SEQ ID NO: 70) |
| Spi1 | ATGTTACAGGCGTGCAAAATGG | TGATCGCTATGGCTTTCTCCA |
| | (SEQ ID NO: 71) | (SEQ ID NO: 72) |
| Gata1 | TGGGGACCTCAGAACCCTTG | GGCTGCATTTGGGGAAGTG |
| | (SEQ ID NO: 73) | (SEQ ID NO: 74) |
| Runx1 | GATGGCACTCTGGTCACCG | GCCGCTCGGAAAAGGACAA |
| | (SEQ ID NO: 75) | (SEQ ID NO: 76) |
| Myod1 | CCACTCCGGGACATAGACTTG | AAAAGCGCAGGTCTGGTGAG |
| | (SEQ ID NO: 77) | (SEQ ID NO: 78) |
| Myog | GAGACATCCCCCTATTTCTACCA | GCTCAGTCCGCTCATAGCC |
| | (SEQ ID NO: 79) | (SEQ ID NO: 80) |
| Myf5 | AAGGCTCCTGTATCCCCTCAC | TGACCTTCTTCAGGCGTCTAC |
| | (SEQ ID NO: 81) | (SEQ ID NO: 82) |
| Adipoq | TGTTCCTCTTAATCCTGCCCA | CCAACCTGCACAAGTTCCCTT |
| | (SEQ ID NO: 83) | (SEQ ID NO: 84) |
| Pparg | TCGCTGATGCACTGCCTATG | GAGAGGTCCACAGAGCTGATT |
| | (SEQ ID NO: 85) | (SEQ ID NO: 86) |
| Lipe | CCAGCCTGAGGGCTTACTG | CTCCATTGACTGTGACATCTCG |
| | (SEQ ID NO: 87) | (SEQ ID NO: 88) |
| Sox5 | AGCCGCAATGCAGGTTTCT | TTGTGCTCTTGTCTGTGTGAAT |
| | (SEQ ID NO: 89) | (SEQ ID NO: 90) |
| Sox9 | GAGCCGGATCTGAAGAGGGA | GCTTGACGTGTGGCTTGTTC |
| | (SEQ ID NO: 91) | (SEQ ID NO: 92) |
| Acan | CCTGCTACTTCATCGACCCC | AGATGCTGTTGACTCGAACCT |
| | (SEQ ID NO: 93) | (SEQ ID NO: 94) |
| Actb | TTCTTTGCAGCTCCTTCGTT | ATGGAGGGGAATACAGCCC |
| | (SEQ ID NO: 95) | (SEQ ID NO: 96) |
| Rpl7 | TCTCTCTTCTTTTCCGGCTG | TTCTTGAGGGTTTCTGGCAC |
| | (SEQ ID NO: 97) | (SEQ ID NO: 98) |

For RT-PCR analyses of clonal assays, total RNA of a single ieCPC clone was collected and reverse-transcribed using the Power SYBR® Green Cells-to-Ct™ Kit (Life Technologies). PCR reactions were performed using the Taq PCR Kit (New England Biolabs). The housekeeping gene Actb was used as an internal control.

### Immunofluorescence Staining Analysis

To analyze intracellular markers, cells were fixed with 4% paraformaldehyde (Sigma-Aldrich) for 15 min, washed three times with phosphate buffered saline (PBS), and incubated in PBS containing 0.3% Triton X-100 (Sigma-Aldrich) and 3% IgG-free BSA (Jackson ImmunoResearch) for 1 hour at room temperature. To analyze cell-surface markers, such as CD31 and VE-Cadherin, cells were grown on matrigel-coated glass coverslips (Warner Instruments) and fixed with pre-chilled acetone for 2 min, rehydrated, washed three times with PBS, and incubated in PBS containing 3% IgG-free BSA for 1 hour at room temperature. Primary antibodies were incubated overnight at 4° C.

The primary antibodies employed included Gata4 (sc-25310, Santa Cruz Biotechnology; 1:200); Mef2c (5030, Cell Signaling Technology; 1:200); Isl1 (39.4D5, Developmental Studies Hybridoma Bank; 1:50); Nkx2-5 (ab35842, Abcam; 1:50); Ki-67 (550609, BD Biosciences; 1:200); cardiac troponin T (cTnT) (MS-295-P1, Thermo Scientific; 1:400); myosin (MF20, Developmental Studies Hybridoma Bank; 1:400); α-actinin (A7732, Sigma-Aldrich; 1:400); myosin light chain (MLC) 2v (10906-1-AP, ProteinTech Group; 1:200); MLC2a (310 11, Synaptic Systems; 1:400); myosin heavy chain (MHC) (ab15, Abcam; 1:500); cardiac troponin I (cTnI) (SC-15368, Santa Cruz Biotechnology; 1:200); α-smooth muscle actin (α-SMA) (A-2547, Sigma-Aldrich; 1:800); calponin (C-2687, Sigma-Aldrich; 1:800); cluster of differentiation 31 (CD31) (BD-550274, BD Biosciences; 1:50); vascular endothelial (VE-)cadherin (SC-9989, Santa Cruz Biotechnology; 1:50); myogenin (SC-12732, Santa Cruz Biotechnology; 1:200); Oct4 (SC-5279, Santa Cruz Biotechnology; 1:500); Nanog (ab80892, Abcam; 1:500); Sox2 (AB5603, EMD Millipore; 1:500), and CD45 (BD-550539, BD Biosciences; 1:50).

After washing cells three times with PBS, the cells were incubated with isotype-matched Alexa Fluorescence-conjugated secondary antibodies (Invitrogen) for 1 hour at room temperature. Cell nuclei were stained with DAPI (D9542, Sigma-Aldrich). Images were acquired with a Zeiss Axio Observer microscope equipped with an Axiocam HRm camera and processed with Axiovision 4.7.1 software. The expression of cTnT (measured by multiplying staining intensity and area) and Isl1 or α-SMA (percentage in total cells) were monitored and analyzed by an IN Cell Analyzer 2000.

For immunostaining in cell-transplantation studies, heart samples were collected two weeks after transplantation, fixed in 0.4% paraformaldehyde overnight, dehydrated in 20% sucrose (Sigma-Aldrich), embedded in OCT compound (VWR International), and frozen in dry ice-conditioned isopentane. Heart samples were cut vertically in 8-μm sections and stained as described above.

### Flow Cytometry and Sorting

To detect cell-surface markers, dissociated cells were incubated with antibodies including Phycoerythrin (PE)-conjugated Flk-1 (12-5821, eBioscience; 1:50). Allophycocyanin (APC)-conjugated-PdgfR-α (17-1401, eBioscience; 1:100), PE-conjugated c-Kit (BD-553869, BD Biosciences; 1:100), APC-conjugated CD45 (BD-559864, BD Biosciences; 1:100), or APC-conjugated CD31 (17-0311, eBioscience; 1:100) antibodies at room temperature for 1-1.5 hr. Isotype-matched normal IgGs were used as negative controls.

To detect intracellular antigens, dissociated cells were fixed and permeabilized with a BD Cytofix/Cytoperm™ Fixation/Permeabilization Kit (BD Biosciences), and then incubated with antibodies for cTnT (MS-295-P1 Thermo Scientific; 1:200) and α-SMA (A-2547, Sigma-Aldrich; 1:200) at 4° C. overnight. After three washes, cells were incubated with isotype-matched Alexa Fluorescence-conjugated secondary antibodies (Invitrogen) for 1 hour at room temperature and detected by an LSR II Flow Cytometer (BD Biosciences).

For sorting, dissociated cells were incubated with PE-conjugated Flk-1 antibody (1:25) and APC-conjugated PdgfR-α antibody (1:50) for 2 hours at 4° C. with rotation. Flk-1⁺/PdgfR-α⁺ cells were sorted with an Aria III Cell Sorter (BD Biosciences). Isotype-matched normal IgGs served as negative controls.

### RNA Sequencing and Alignment

Total RNA was prepared using the RNeasy Plus Mini Kit with Qiashredder columns (Qiagen). Potentially residual DNA was removed by on-column DNase digestion with RNase-free DNase (Qiagen). Total RNA (10 μg) was used as input material for preparing the RNA sequencing (RNA-seq) libraries with an Ovation Ultralow System V2 (NuGEN). Amplified libraries were sequenced on HiSeq 2500.

Paired-end RNA-Seq reads were aligned to the reference assembly mm9 with Tophat 2.0.13 (Kim et al., 2013). Aligned reads were assigned to genes using “featureCounts” (Liao et al., 2014), part of the Subread suite (see website at subread.sourceforge.net/). Differential expression P-values were calculated using edgeR (Robinson et al., Bioinformatics 26: 139-140 (2010)), an R package available through Bioconductor. Genes without at least two samples with a CPM (counts per million) value between 0.5 and 5000 were filtered out. Remaining expression values were normalized using “calcNormFactors” in edgeR. P-values for expression differences between samples were then calculated in edgeR with a negative binomial distribution for gene expression. Finally, FDR (false discovery rate) for each P-value was calculated by the built-in R function “p.adjust” using the Benjamini-Hochberg method. For hierarchical cluster analyses, Cluster 3.0 software was used. For gene-ontology analyses of gene-set enrichment among different samples, the DAVID Functional Annotation Tool was used (see website at david.abcc.ncifcrf.gov/tools.jsp).

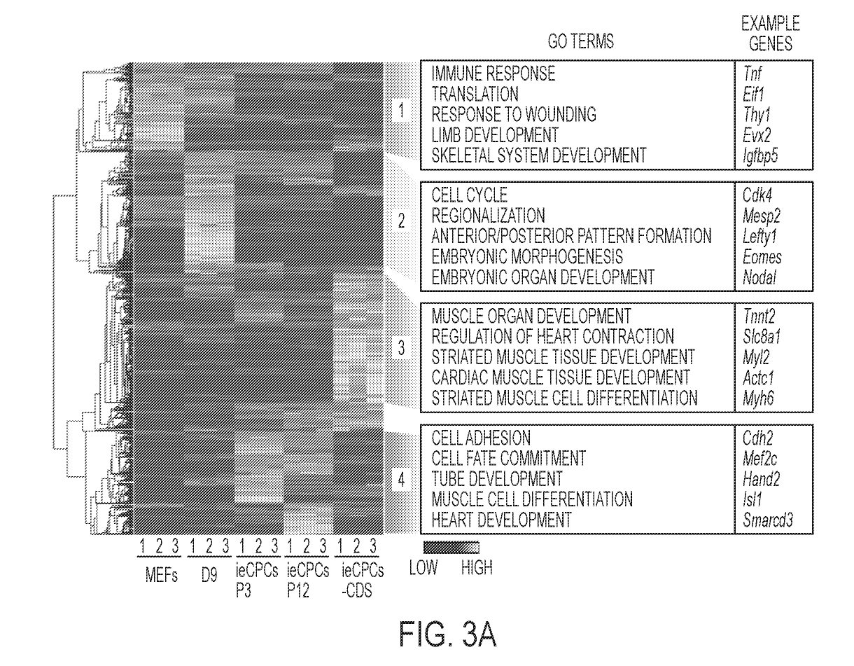

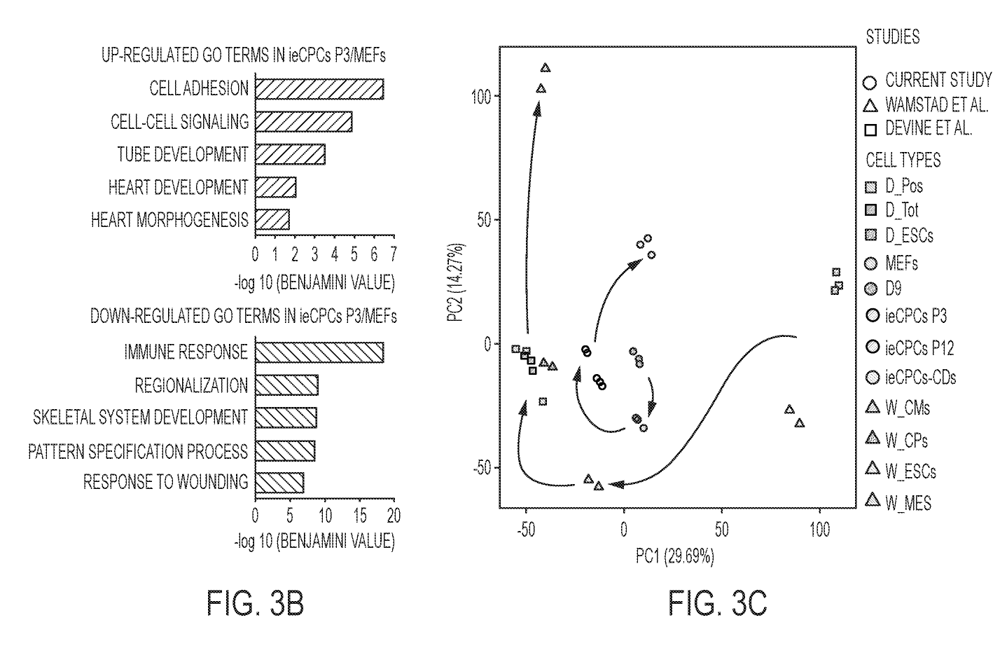

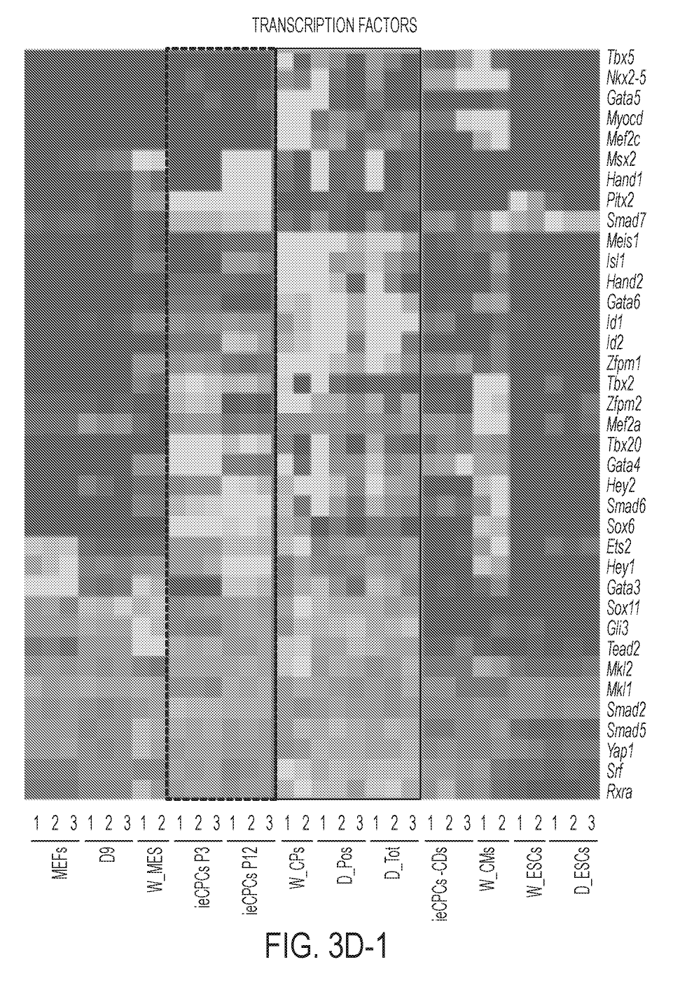

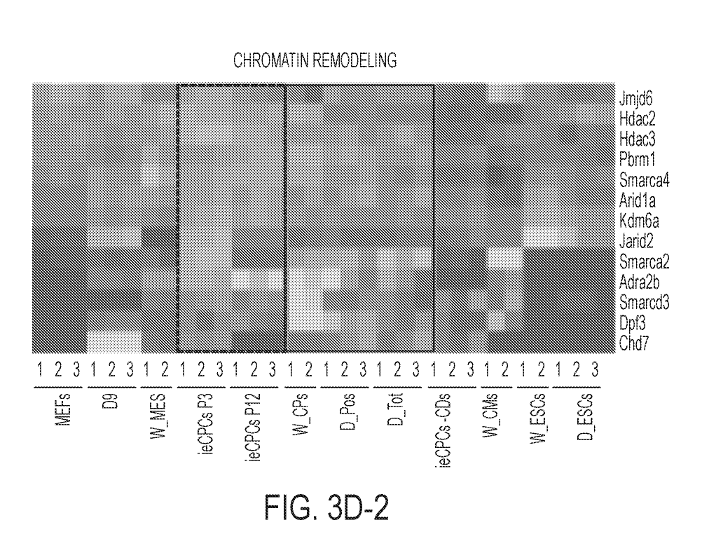

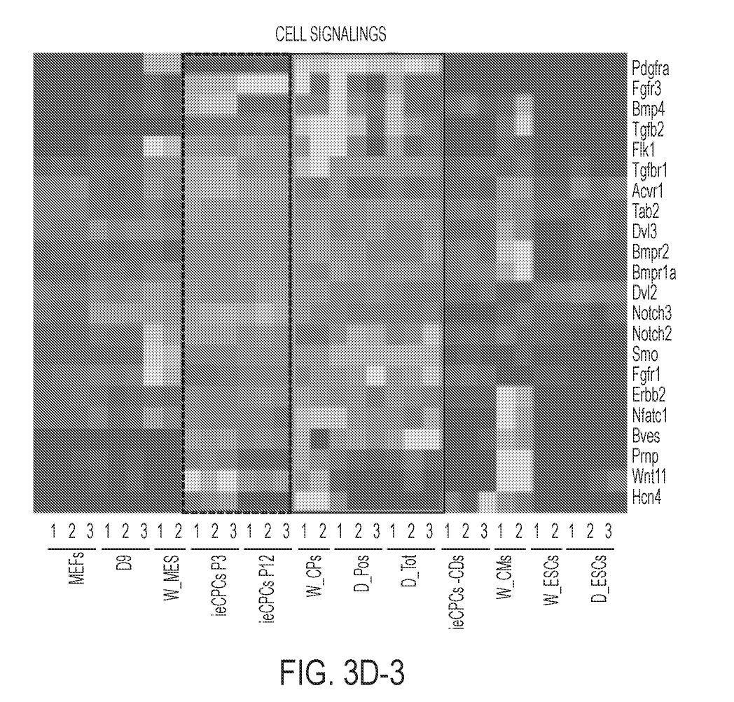

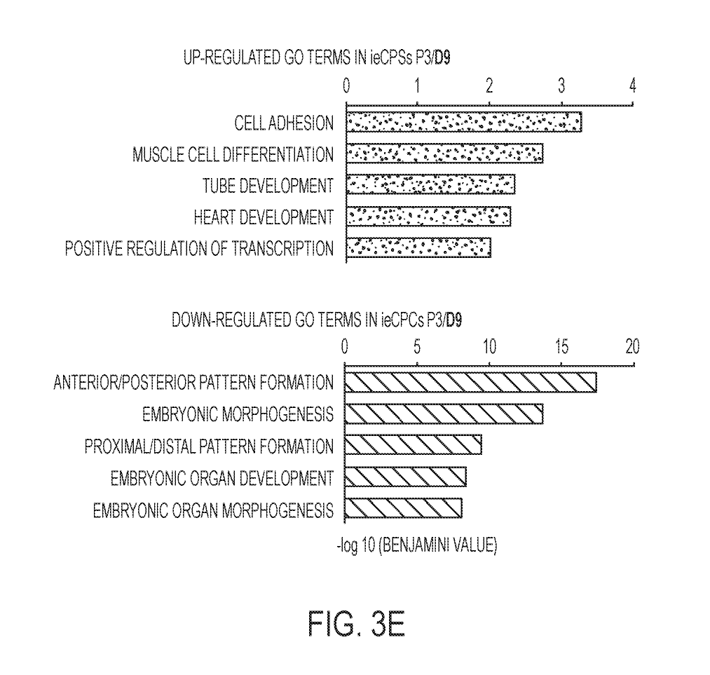

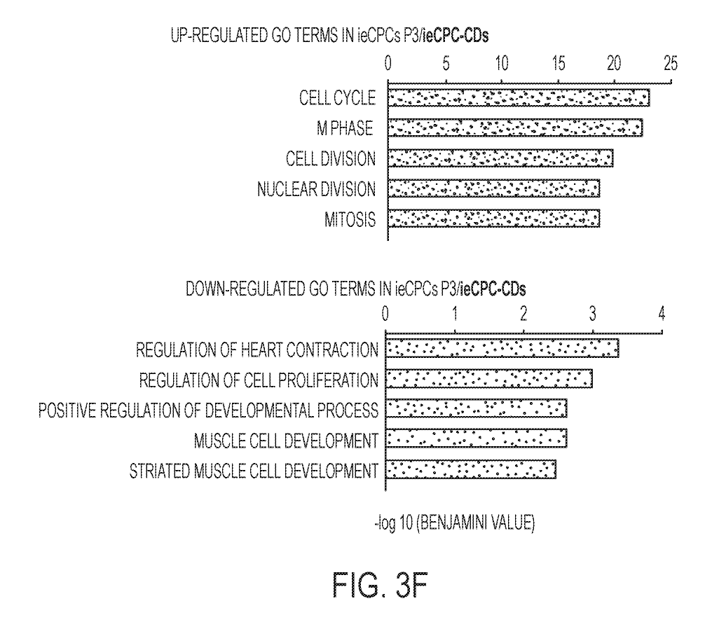

Samples in this work were normalized using the RUVseq (Risso et al., Nature biotechnology 32: 896-902 (2014)) bioconductor package in R. Genes with five or less raw counts in, at most, two samples were filtered out from the analyses. From the remaining set of genes, coefficients of variation were computed for each gene across all samples within each study. One-thousand genes with the lowest mean coefficient of variation of expression across the three studies were then chosen as control genes. Two factors (k=2) were supplied to the RUVg function to remove the unwanted variation associated with the lab of origin for each sample. plotPCA function was used to create the principal component-analyses plot in FIG. 3C.

### Transmission Electron Microscopy

Transmission electron microscopy was performed using methods described by Fu et al. Stem Cell Reports 1: 235-247 (2013)). Briefly, cells were fixed in 2% glutaraldehyde and 1% paraformaldehyde in 0.1M sodium cacodylate buffer, pH 7.4, post-fixed in 2% osmium tetroxide in the same buffer, en-block stained with 2% aqueous uranyl acetate, dehydrated in acetone, infiltrated, and embedded in LX-112 resin (Ladd Research Industries, Burlington, Vt.). Embedded samples were ultrathin sectioned on a Reichert Ultracut S ultramicrotome and counter stained with 0.8% lead citrate. Grids were examined on a JEOL JEM-1230 transmission electron microscope (JEOL USA, Inc.) and photographed with the GatanUltrascan 1000 digital camera (Gatan Inc.).

### Electrophysiology and Intracellular Ca²⁺ Measurements

Contracting cardiomyocytes derived from ieCPCs (ieCPC-CMs) were dispersed by first treating cells with Collagenase B for 5-10 min and then with Accutase for 5 min. Dissociated cells were replated onto matrigel-coated coverslips (Warner Instruments). After visible beating was reconfirmed, the coverslips were loaded with the Ca²⁺-sensitive fluorescent indicator Fluo-4 (see below), and then placed in a superfusion bath (Warner Instruments) on a Nikon TiS inverted microscope equipped with a microfluorometer (lonOptix). Superfusion solutions were warmed to 30° C. with a superfusion system and heated perfusion pencil (ValveLink, AutoMate Scientific).

Single, spontaneously contracting myocytes as well as small clusters of cells were selected for study, with one cell under amphotericin B-perforated patch clamp (Spencer et al. 2014). Briefly, patch electrodes of approximately 2 MΩ (WPI) were tip-filled by dipping (20 s) in an intracellular solution containing (in mM): KCl 120, NaHEPES 20, MgATP 10, K₂EGTA 5, MgCl₂ 2, pH-adjusted to 7.1 with KOH, and then back filled with the same solution containing amphotericin B (240 μg/ml). Coverslips were superfused at a constant flow (Warner Instruments) with modified Tyrode's extracellular solution containing (mM): NaCl 137, NaHEPES 10, dextrose 10, KCl 5, CaCl₂ 2, and MgCl₂ 1, set to pH 7.4 with NaOH. Spontaneous action potentials (APs) were detected in current-clamp mode with zero applied current and digitized for 30 s per data file.

The ieCPC-CMs on coverslips were loaded with Fluo-4 AM in a 1:10 mixture of the indicator dissolved in dry dimethyl sulfoxide at 5 mM, plus Powerload concentrate (Life Technologies). This mixture was diluted 100-fold into extracellular Tyrode's solution and substituted for culture medium in dishes containing coverslips (final indicator concentration, 5 μM). Cells were loaded with dye for 20 min at room temperature and placed in dye-free extracellular solution for 20 min to allow for indicator de-esterification before recordings were taken. Fluo-4 fluorescence transients were recorded with a standard filter set with excitation and emission centered on 480 nm and 535 nm, respectively (Chroma Technology).

Fluorescence was obtained from contracting cells plus a cell-free border using a cell-framing adaptor (IonOptix). Background fluorescence, recorded after removing the cell(s) from the field of view, was subtracted from all recordings. Between sampling periods, the excitation light was blocked with a shutter (Vincent Associates). Background-corrected fluorescence transients were expressed relative to the diastolic fluorescence between spontaneous APs and were considered to reflect underlying changes in intracellular Ca²⁺ concentration. Unless stated, reagents were from Sigma Chem. Co. Pharmacological studies were conducted on the spontaneous fluorescence transients of non-patch-clamped myocytes, and, to maximize coverslip usage, pharmacological agents were applied locally (only to the cell or cluster of interest) with the perfusion pencil.

APs and fluorescence transients were digitized at 5 kHz and low-pass filtered at 2 kHz. The maximum upstroke velocity of the AP (Vₘₐₓ) was calculated with pClamp software (Molecular Devices). AP durations, determined from Vₘₐₓ, were calculated for every AP in a given data file using in-house analysis routines implemented in Excel 2007 (Microsoft). Voltages were corrected for a −5.6 mV liquid junction potential.

### Functional Assays of Endothelial Cells in Vitro

To analyze tube formation on matrigel in vitro, cells were seeded on top of a thin layer of matrigel at a density of 2.5×10⁴ cells/well of a 12-well plate. After 24 hours, cells were subjected to brightfield imaging microscopy to visualize formation of tube-like structures. Uptake of acetylated low-density lipoprotein (ac-LDL) was assessed by incubating cells with 5 μg/ml of ac-LDL conjugated with Alexa Fluor-594 (Invitrogen) for 4 hr. After incubation, cells were washed with PBS before fluorescence microscopy.

### Contractile Assay of Smooth Muscle Cells in Vitro

ieCPCs, ieCPC-SMCs, and primary SMCs were treated with carbachol (100 μM, Sigma-Aldrich) and monitored with a Zeiss Axio Observer microscope in a time-lapse manner at 10-minute intervals for 1 hour. Cell-surface areas were then calculated with ImageJ software.

### Clonal Assay

Single ieCPCs were isolated by FACS, plated onto 384-well plates at one cell per well, and cultured for 3 days in SFD medium supplemented with 20% FBS; 1 μM thiazovivin (Stemgent) was added into the culture medium to improve ieCPCs survival. After overnight culture, each well of the plate was monitored with a microscope and rare wells that contained more than one cell were excluded from experiments. At day 4, medium was changed to SFD supplemented with 10 ng/ml FGF2, 100 ng/ml IGF1, 10 ng/ml EGF, and 250 μM ascorbic acid to promote cell growth. Colonies were scored after 28 days of culture.

### In Vivo Transplantation

Myocardial infarction was induced by permanently ligating the left anterior descending artery in 10-12-week-old male NOD.Cg-Prkdcscid Il2rgtm1Wjl/SzJ (NSG) mice (The Jackson Laboratory) as described by Qian et al. (Nature 485: 593-598 (2012)). ieCPCs and their parental 2^{nd} MEFs were labeled by retrovirus harboring an RFP reporter and transplanted into infarcted hearts. One million donor cells were injected along the boundary between the infarct and border zones immediately after coronary ligation.

For teratoma-formation experiments, mESCs (0.5 million cells, n=2) and ieCPCs (1 million cells, n=6) were similarly injected into the infarcted hearts of NSG immunodeficient mice that were 10-12-weeks-old.

All mouse work was conducted according to the Guide for the Care and Use of Laboratory Animals, as adopted by the National Institutes of Health, and with approval of the University of California, San Francisco Institutional Animal Care and Use Committee.

### Echocardiography

Echocardiography was performed with the Vevo 770 High-Resolution Micro-Imaging System (FUJIFILM VisualSonics) with a 15-MHz linear-array ultrasound transducer as described previously (Qian et al., 2012). Briefly, M-mode tracing with a sweep speed of 50 mms⁻¹ at the papillary muscle level was used to measure left-ventricular anterior and posterior wall thickness. These data were then used for calculating the shortening fraction. B-mode was used for two-dimensional measurements of end-systolic and end-diastolic dimensions, which were obtained for calculating the ejection fraction. All surgeries, cell transplantation, and subsequent analyses were performed blinded and decoded only at the end of the experiments.

### Determination of Scar Size

At the end of the experiments, mice were anaesthetized by 5% isoflurane, and 0.1M KCl was injected into mouse hearts to stop them at diastole. The hearts were then fixed, cut vertically in 5-μm sections, and further processed for histology analyses.

To determine the scar size, Masson-Trichrome staining was performed following a standard protocol (Qian et al., 2012) on hearts 12 weeks after coronary artery ligation. For each group, three representative hearts that had a shortening fraction similar to the average value of the group were collected. Then the scar size was measured with ImagePro software to calculate the percentage of the total left-ventricular area (red plus blue) showing fibrosis (blue). For each sample, scar size was evaluated on transverse sections spanning eight levels and calculated as previously described (Takagawa et al., 2007). From each level, two slices of tissue were measured as technical repeats (for a total of 48 sections).

### Statistical Analysis

Data were presented as means±S.E. Statistical significance of differences was estimated by student's t-test in Microsoft Excel. Differences were considered significant when P-value<0.05.

### Accession Numbers

RNA sequencing data were deposited in NCBI's Gene Expression Omnibus (GEO) database and can be accessed by the accession number GSE77375.

### Example 2: Screening to Capture ieCPCs

To develop effective and personalized cell therapies, CPCs from an easily accessible source were needed. To this end, cell-activation and signaling-directed (CASD) lineage conversion was used, which transiently exposes cells to reprogramming factors and small molecules in conjunction with cardiac-inductive signals. See, Efe et al., Nat Cell Biol 13: 215-222 (2011); and Wang et al., Cell Rep 6: 951-960 (2014). Remarkably, cell populations were observed that expressed markers corresponding to different stages of cardiogenesis during reprogramming, potentially helping to identify and capture the desirable ieCPCs.

To capture and expand putative ieCPCs arising during CASD-lineage conversion, combinations of cardiogenic signals were screened with a functional assay under the hypothesis that ieCPCs would respond and propagate with the right combination of cardiogenic signaling at optimal strength. Cardiac conversion of secondary mouse embryonic fibroblasts (2^{nd} MEFs) was also performed. First, the cells were activated by transiently expressing reprogramming factors for six days and allowed cardiac specification for another two days. From day 8, the activated cells were treated with various combinations of modulators of Wnt, Activin/Nodal, BMP, FGF, VEGF, PDGF, and Notch pathways at different concentrations for another three days and then these cells were passaged three times under the same conditions, where each passage was four days apart. Spontaneous cardiac differentiation was then assessed in cells that had extensively propagated without showing morphological changes over the serial passages. See FIG. 1A for a schematic diagram of the process employed.

These cells were cultured in defined basal differentiation conditions without serum, knock-out serum replacement, or any other exogenous differentiation cues, which is a key criterion in evaluating the committed cardiovascular fate. Cardiac differentiation of these cells was monitored by the presence of contractile patches and the expression of cardiac troponin T (cTnT), a CM-specific marker. After repetitive testing and optimizing, one promising condition was identified that retained the cardiac potential of induced cells after three passages, indicating that putative ieCPCs had been captured. This condition contained BMP4 (5 ng/ml), Activin A (10 ng/ml), CHIR99021 (3 AM, a glycogen synthase kinase 3 inhibitor), and SU5402 (2 μM, an inhibitor of FGF, VEGF, and PDGF signaling), hereafter referred to as BACS.

### Example 3: ieCPC Characterization

To characterize the molecular qualities of the putative ieCPCs, the expression of cardiac markers was examined by quantitative PCR (qPCR).

Consistent with our hypothesis, a panel of transcription factors known to be enriched in committed CPCs—Gata4, Mef2c, Tbx5, and Nkx2-5—were highly expressed in the expanded cells (see FIG. 1B), whereas differentiated CM markers—Tmu2 and Myh6—were barely detectable (data not shown). Interestingly, Flk1 and Pdgfra, which encode the two CPC surface markers Flk-1 and PdgfR-α, were also highly enriched in expanded ieCPCs (FIG. 1B). When expressed simultaneously, these two markers identify CPCs committed to a cardiovascular lineage (see, Hirata et al., J Biosci Bioeng 103: 412-419 (2007); Kattman et al., Cell Stem Cell 8: 228-240 (2011)).

The cell responses to BACS treatment were examined by analyzing Flk-1 and PdgfR-α expression with fluorescence-activated cell sorting (FACS). Flk-1⁺/PdgfR-α⁺ cells first appeared at day 8, responded to BACS treatment, and dominated the total population (more than 70%) by one week later. However, without BACS, these cells did not grow and eventually attenuated (FIGS. 1C and 1D).

To distinguish whether the initial BACS treatment increased Flk-1⁺/PdgfR-α⁺ cells through a mechanism of induction and/or proliferation, 5-ethynyl-2′-deoxyuridine (EdU) incorporation was evaluated in transdifferentiating cells to monitor DNA synthesis in cell culture. The EdU was added to the culture medium with BACS at day 8 and the percentage of EdU⁺/Flk-1⁺/PdgfR-α⁺ cells was examined at day 14. About 33% of Flk-1⁺/PdgfR-α⁺ cells did not incorporate EdU, indicating that they were generated through direct induction and not proliferation. To determine if proliferation is needed to enrich Flk-1⁺/PdgfR-α⁺ cells, the cells were treated with the cell-cycle inhibitor NU6140 (4 μM) along with BACS starting on day 8. The ratio of FIk-1⁺/PdgfR-α⁺ cells dramatically decreased at day 14. These data demonstrate that mechanisms of both cellular induction and proliferation contribute to the generation of Flk-1⁺/PdgfR-α⁺ cells after initial BACS treatment.

Next, the three subpopulations of cells (i.e., Flk-1⁺/PdgfR-α⁺, Flk-1⁻/PdgfR-α⁺, and Flk-1⁻/PdgfR-α⁻) were isolated on day 13 by FACS and their cardiogenic potentials were examined. Only Flk-1⁺/PdgfR-α⁺ cells efficiently gave rise to cTnT⁺ CMs (FIGS. 1E, IF, 1J) and formed spontaneously beating clusters. Flk-1⁺/PdgfR-α⁺ cells that were cultured for two passages in BACS to recover from the initial sorting stress showed more robust cardiac differentiation potential (FIG. 1G). These data demonstrate that Flk-1⁺/PdgfR-α⁺ cells, but not other subpopulations, represent the putative ieCPCs in BACS-treated cells.

### Example 4: ieCPCs are Commited to a Cardiovascular Fate

The Flk-1⁺/PdgfR-α⁺ ieCPCs were examined to ascertain whether these cells truly were committed cardiovascular precursors or were more primitive CPCs that require specific signals and steps to be further differentiated. The inventors found that treating ieCPCs with BMP4 significantly impaired their cardiac differentiation. However, treating the cells with the Wnt inhibitor IWP2 (5 μM), which promotes CM specification only from late-stage CPCs (Burridge et al., 2012), dramatically enhanced cellular differentiation (FIGS. 1E, IF, and 1K). Even in basal differentiation conditions without any specific induction signaling molecules, Flk-1⁺/PdgfR-α⁺ cells directly differentiated into all three cardiovascular lineages, including CMs (cTnT⁺/cTnI⁺), ECs (CD31⁺/VE-cadherin⁺), and SMCs (α-smooth muscle actin (α-SMA)⁺/calponin⁺) (FIG. 1H). These distinct responses show that ieCPCs have already committed to a cardiovascular fate. In addition, ieCPCs highly expressed several committed CPC markers, including Gata4, Mef2c, Isl1, and Nkx2-5, and the proliferative marker Ki-67 (FIG. 1); whereas uncommitted mesoderm genes were only transiently expressed at earlier stages during the generation of ieCPCs (FIG. 1L-1M).

Next, the inventors determined whether ieCPC differentiation was restricted to a cardiovascular fate. Gene expression of mesoderm-derived non-cardiovascular lineages was evaluated for ieCPCs that underwent either CM-, EC-, and SMC-specific differentiation or non-specific differentiation induced by fetal bovine serum (FBS) for 10 days. Under these conditions, no induction of non-cardiovascular genes was observed, including no detectable levels of markers of hematopoietic precursors, skeletal muscles, adipocytes, and chondrocytes.

To further confirm their restricted cardiovascular potentials, ieCPCs were cultured in the induction conditions specified for each non-cardiovascular mesodermal lineage and the differentiation of ieCPCs was compared to mesodermal cells derived from mouse embryonic stem cells (mESCs). In differentiated ieCPCs, c-Kit⁺/CD45⁺ hematopoietic progenitors or more-differentiated c-Kit⁻/CD45⁺ hematopoietic cells were rarely detected. In contrast, mESC-derived mesodermal cells routinely generated those hematopoietic cells. Consistently, ieCPCs exhibited limited or no potential to differentiate into myogenin⁺/MHC⁺ skeletal muscles, oil red-O⁺ adipocytes, or alizarin red-S⁺ chondrocytes when compared with mESC-derived mesodermal cells, even when the ieCPCs were exposed to each lineage-specific induction cue.

To more precisely characterize cells after ieCPC differentiation, the percentage of CMs (cTnT⁺), ECs (CD31⁺), and SMCs (α-SMA⁺) was analyzed by FACS in CM-, EC-, SMC-, and FBS-differentiation conditions. Isl1, a well-recognized CPC marker that diminishes as soon as CPCs enter a differentiation program (Moretti et al., 2006), was used to trace undifferentiated ieCPCs. The inventors found that most (>93%) of the cells in each differentiation condition were either CMs, ECs, SMCs, or undifferentiated ieCPCs, confirming the restricted cardiovascular potentials of ieCPCs. Notably, CM generation was not detected when ieCPCs were differentiated in FBS-containing conditions at a low seeding density (1×10⁴ cells/cm²), which is suitable for inducing most mesodermal lineages. This deficiency can be prevented by using the cell seeding density optimal for CM differentiation (3×10⁵ cells/cm²) and/or by using BMP/Wnt inhibitors.

### Example 5: ieCPCs Can Be Expanded Long-Term

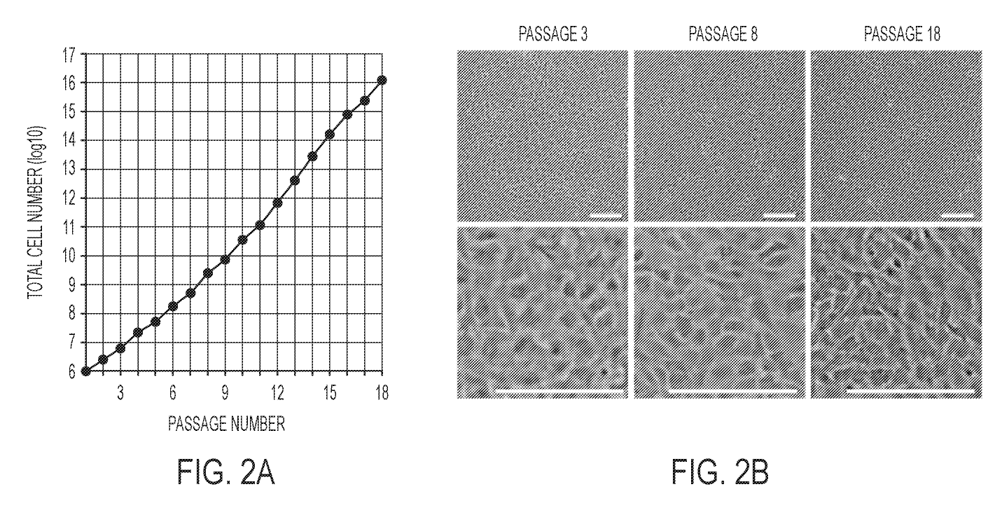

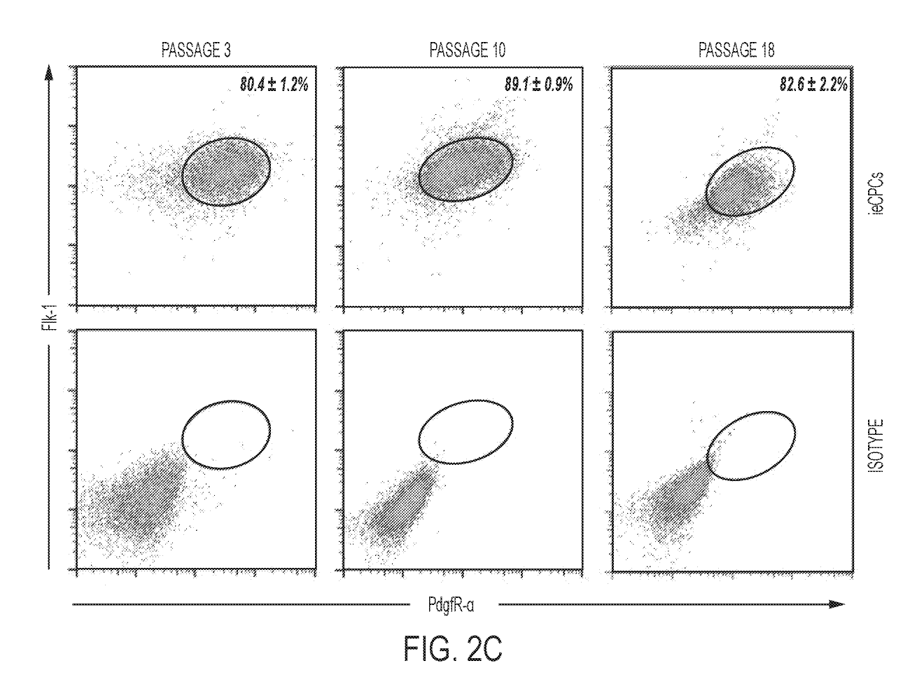

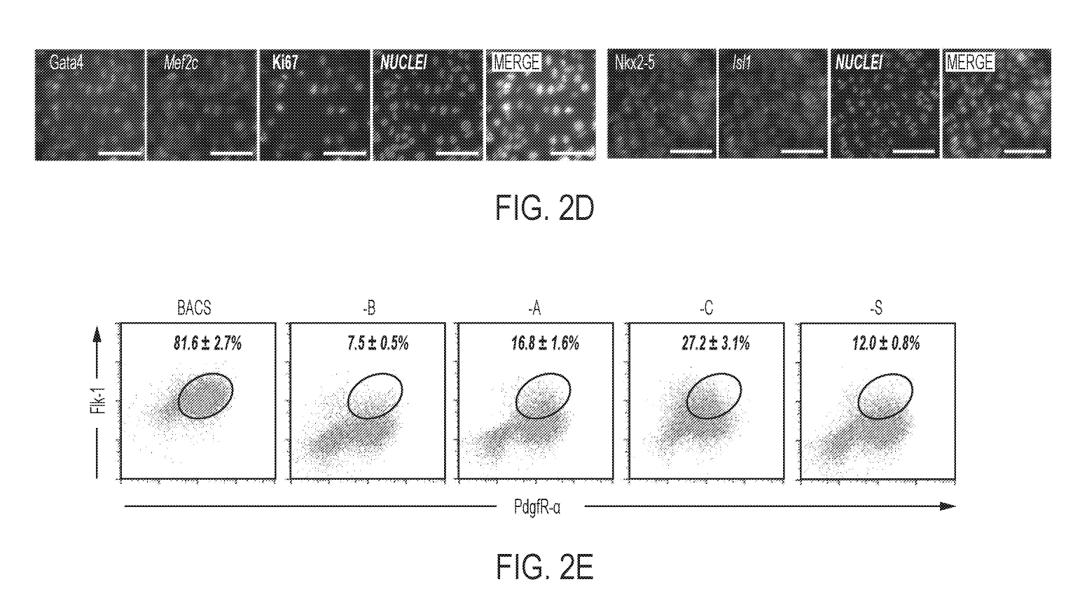

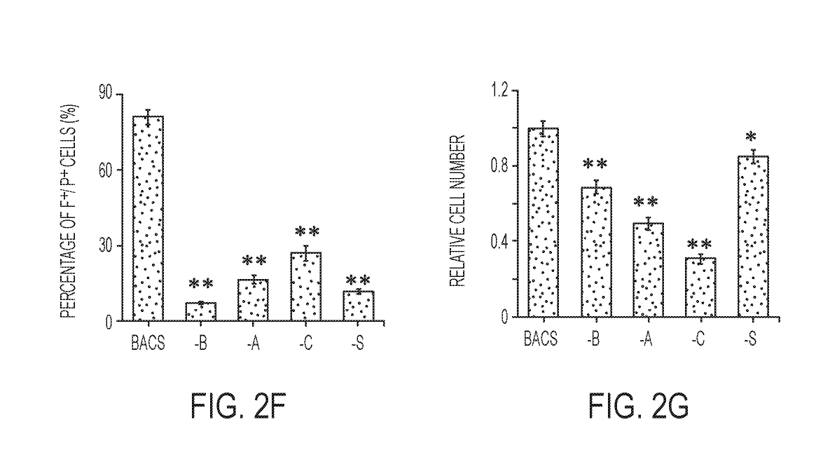

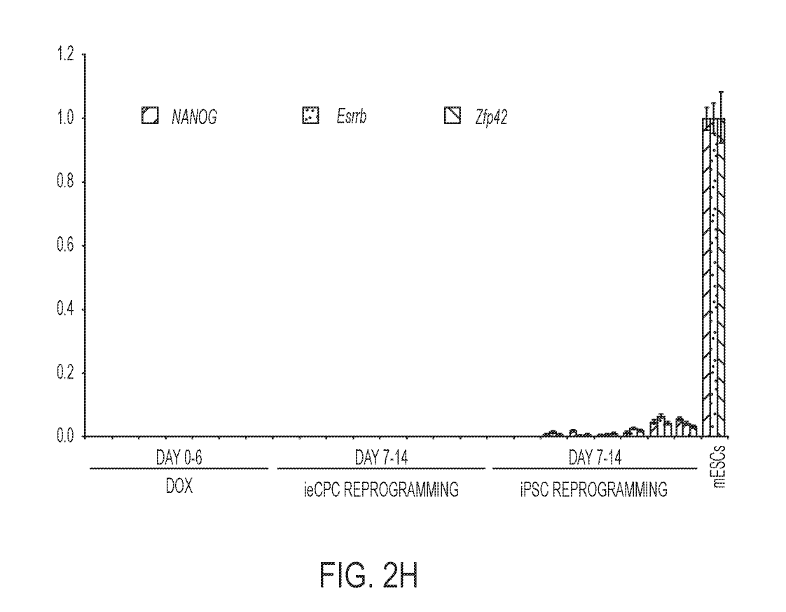

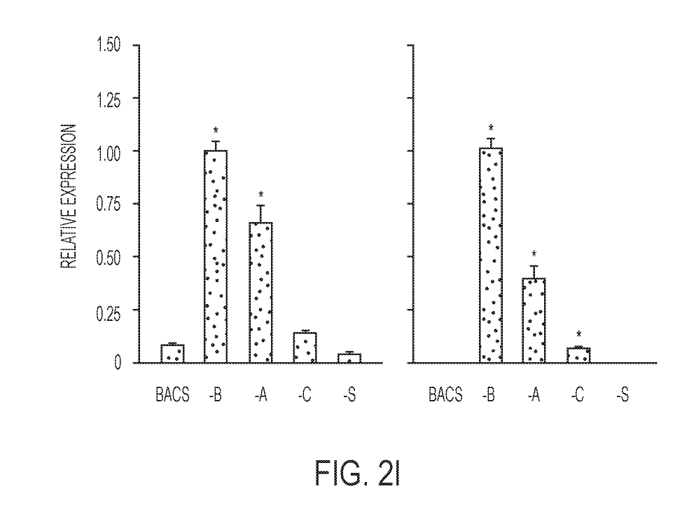

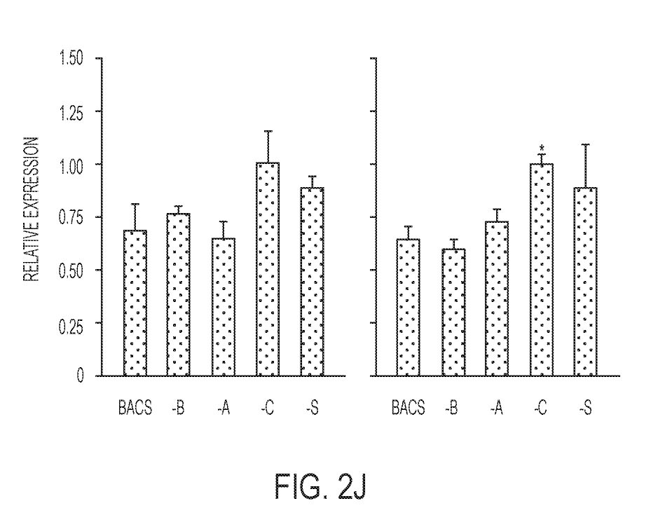

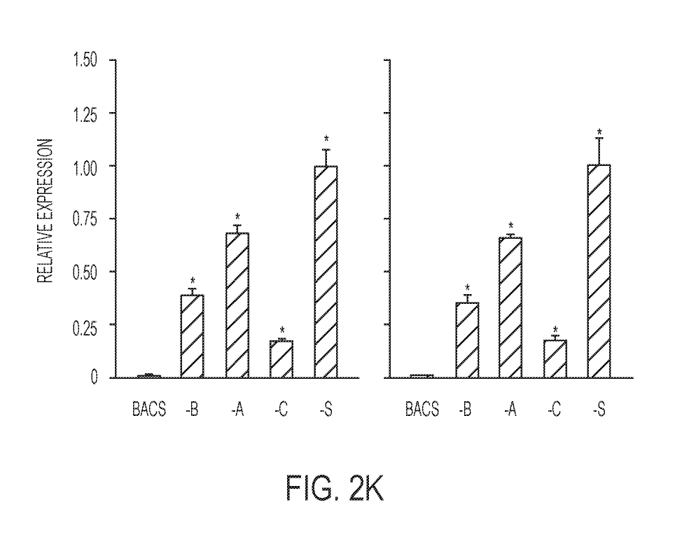

Flk-1⁺/PdgfR-α⁺ ieCPCs were purified by FACS and tested to ascertain whether the cells could be expanded in long-term culture. Purified ieCPCs exhibited normal undifferentiated morphology and could stably propagate in BACS conditions for more than 18 passages (>10¹⁰-fold expansion) (FIG. 2A). To evaluate whether ieCPCs that had been expanded long-term retained their original properties, ieCPCs of early (<5), middle (5-10), and late (>10) passages were compared. At these passage times, the inventors observed that the ieCPCs maintained a high degree of similarity in growth rate (FIG. 2A), undifferentiated morphology (FIG. 2B), and expression of both Flk-1 and PdgfR-α (FIG. 2C) over time. By immunostaining, the inventors observed that the ieCPCs similarly expressed Gata4, Mef2c, Isl1, Nkx2-5, and Ki67 at early, middle, and late passages (FIG. 2D). However, the ieCPCs did not express CM-, EC-, SMC-markers, and pluripotency-related markers (FIG. 2H), even after long-term expansion.

To determine if each component in BACS was required for Flk-1⁺/PdgfR-α⁺ cells to self-renew, each cytokine or chemical was individually omitted and cell expansion was evaluated. Omitting any component of BACS dramatically reduced the percentage of Flk-1⁺/PdgfR-α⁺ cells (FIGS. 2E and 2F) and cell proliferation (FIG. 2G) within three passages, suggesting that each component is indispensable. Removing each component of the BACS cocktail caused SMC-, EC-, or CM-marker genes to accumulate (FIG. 2I-2K), indicating that some cells spontaneously differentiated. These results show that each component of BACS synergistically represses cardiovascular differentiation of ieCPCs to sustain their long-term self-renewal.

To examine the global transcriptional profile of ieCPCs, the transcriptomes were compared of early- and late-passage ieCPCs, their parental MEFs, cells at day 9 of reprogramming (D9), and cardiac derivatives from ieCPCs (ieCPC-CDs) by RNA sequencing. Evaluation using hierarchical cluster analyses showed that early- and late-passage ieCPCs had very similar gene-expression profiles and that those profiles clearly differed from other cell populations (FIG. 3A). Gene ontology (GO) analysis demonstrated that genes specifically expressed in ieCPCs were related to cell adhesion and heart-lineage commitment. Pair-wise comparisons of ieCPCs with MEFs, D9, and ieCPC-CDs showed that ieCPCs were mainly enriched with GO terms associated with CPC fate and functions, such as heart development and cell proliferation. Conversely, ieCPCs lacked GO terms involved in functions of other cell types, such as the immune response in MEFs, early embryonic development of germ layers in D9, and myocyte contraction in ieCPC-CDs (FIGS. 3A, 3B, 3I, and 3J). However, comparing early- and late-passage ieCPCs did not yield any GO terms that met the false discovery rate threshold of ≤0.05, suggesting that ieCPCs retained stable transcriptional signatures when expanded long-term.

To determine whether ieCPCs represent a particular stage of cardiac differentiation of ESCs, the ieCPCs were compared with cells at different stages during cardiac differentiation of mESCs, including undifferentiated ESCs, mesodermal cells, CPCs, and differentiated CMs (Wamstad et al., Cell 151: 206-220 (2012), Devine et al., eLife 3 (2014)). The ieCPCs and ESC-derived CPCs had the highest transcriptional similarity when compared with other reference cell types (FIG. 3C) and the ieCPCs represented an intermediate cardiogenic population between uncommitted mesoderm and terminally differentiated cardiovascular cells (FIG. 3C).

Next, a panel of well-studied genes involved in CPC-fate commitment was evaluated. The ieCPCs highly expressed CPC-related genes, including transcription factors, chromatin remodelers, and cell-signaling molecules (FIG. 3D). However, the ieCPCs weakly expressed markers associated with other cell types, including fibroblasts, early mesoderm, endoderm, ectoderm, non-cardiovascular mesoderm, PSCs, epicardial cells, mesenchymal stem cells, and differentiated CMs, ECs, and SMCs, with expression levels no higher than the ESC-derived CPCs described by Wamstad et al. and Devine et al. Collectively, these results demonstrate that ieCPCs possess a global transcriptional pattern similar to that of normal CPCs derived from ESCs.

### Example 6: Long-Term Expanded ieCPCs Exhibit Multi-Lineage Potentials for Cardiovascular Differentiation in Vitro

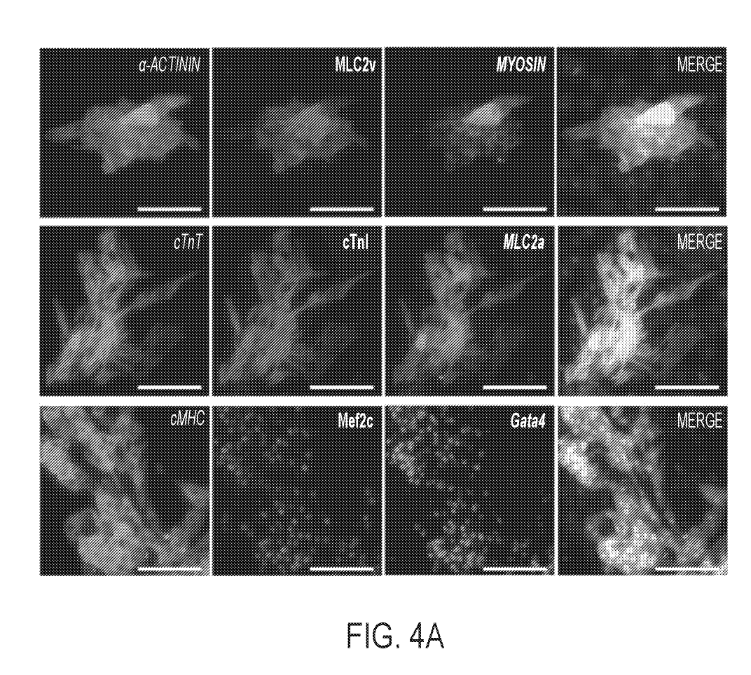

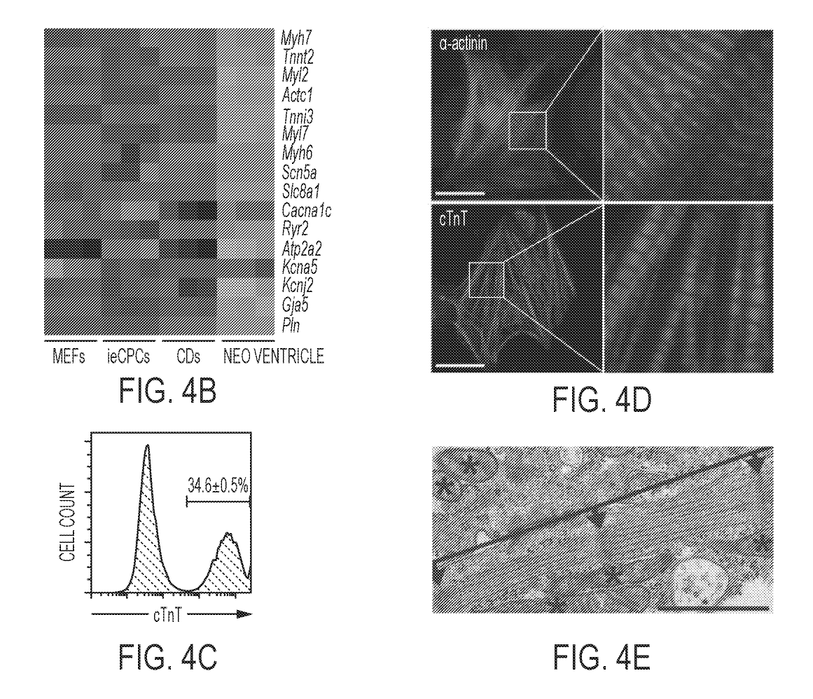

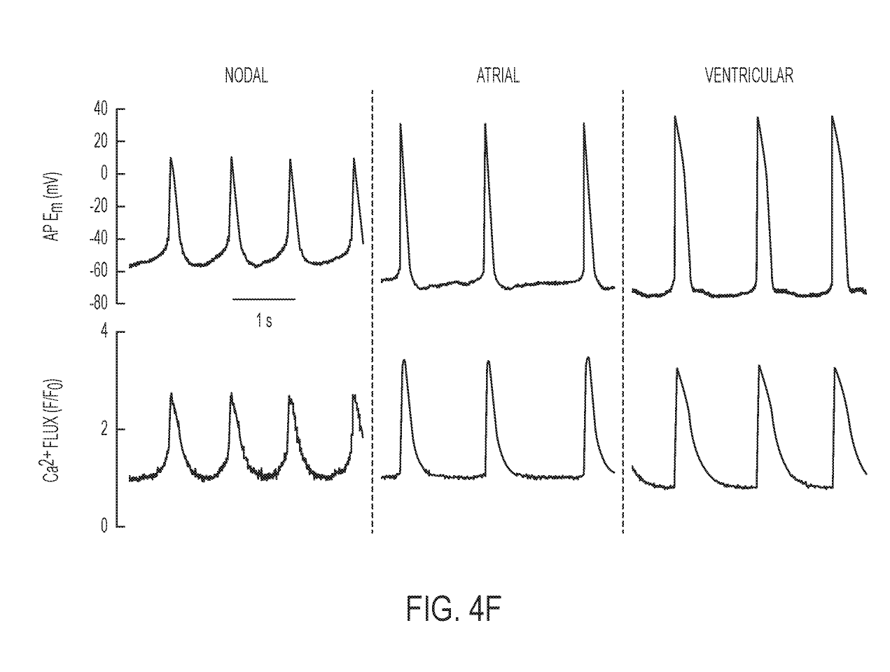

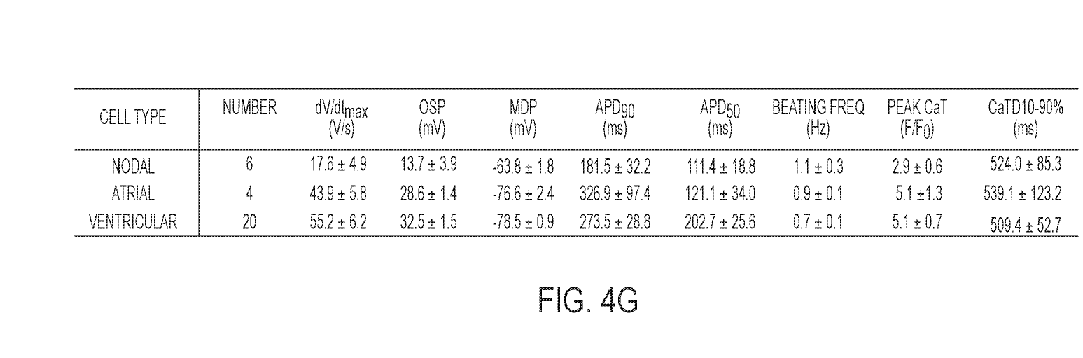

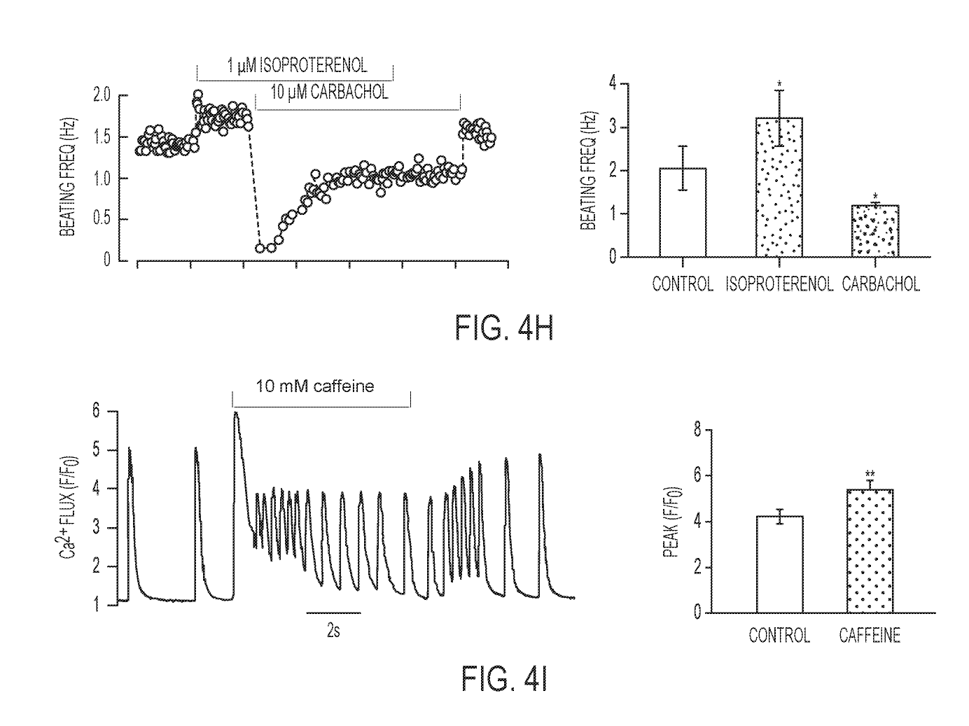

Tests were performed to ascertain whether ieCPCs retain their multi-lineage potential for cardiovascular differentiation when expanded long-term. The ieCPCs were cultured through late passages in differentiation medium supplemented with 1WP2 (5 μM) and monitored their cardiac differentiation daily for spontaneously contracting cells (typically observed first at day 3 of differentiation). The number of beating cells gradually increased until day 10 and remained at a similar level for more than one month. Cardiac differentiation was robust, and synchronized beating sheets formed at day 10. Upon immunostaining, the CMs derived from ieCPCs (ieCPC-CMs) exhibited expression of several CM-specific markers (FIG. 4A). Analysis of gene expression by qPCR demonstrated that many genes important for CM contraction and functional regulation were expressed (FIG. 4B). FACS analysis of cTnT revealed that the efficiency of CM differentiation from ieCPCs was around 35% at day 10 (FIG. 4C). Typically, 10,000 starting ieCPCs produced ˜30,000 total cells, from which we estimate a yield of approximately one CM per input ieCPC.

Next, ieCPC-CMs were further characterized by immunofluorescence of the cardiac-myofilament proteins. As shown in FIG. 4D, single ieCPC-CMs displayed a well-organized sarcomeric structure with clear cross-striations at day 10 (FIG. 4D). This finding was confirmed by transmission electron microscopy, in which the well-organized sarcomeres, myofibrillar bundles, and transverse Z-bands were surrounded by ample mitochondria (FIG. 4E). In addition, intracellular electrical recordings from single beating ieCPC-CMs at day 10 revealed robust action potentials (APs) that were synchronized 1:1 with rhythmic Ca²⁺ transients (FIG. 4F), which closely resembled CMs derived from murine fetal heart or PSCs (Kuzmenkin et al., FASEB J 23, 4168-4180 (2009)). The ratio of AP duration at 90% repolarization (APD₉₀) to APD₅₀ (Kuzmenkin et al., 2009), demonstrated that the ieCPC-CMs had nodal-like (20.0%), atrial-like (13.3%), and ventricular-like (66.7%) APs (FIGS. 4F and 4G). Moreover, 1 μM isoproterenol (a β-adrenergic agonist) significantly increased the frequency of cell contraction and spontaneous Ca⁺ transients. These effects were blocked by 10 μM carbachol, a synthetic muscarinic agonist, suggesting functional and coupled cascades of β-adrenergic and muscarinic signaling in ieCPC-CMs (FIG. 4H). Moreover, 10 mM caffeine elicited large Ca²⁺ transients in ieCPC-CMs (FIG. 4I), indicating the presence of cardiac ryanodine receptors. Thus, the CMs generated from ieCPCs expanded long-term possess physiological features of bona fide CMs and are functional in vitro.

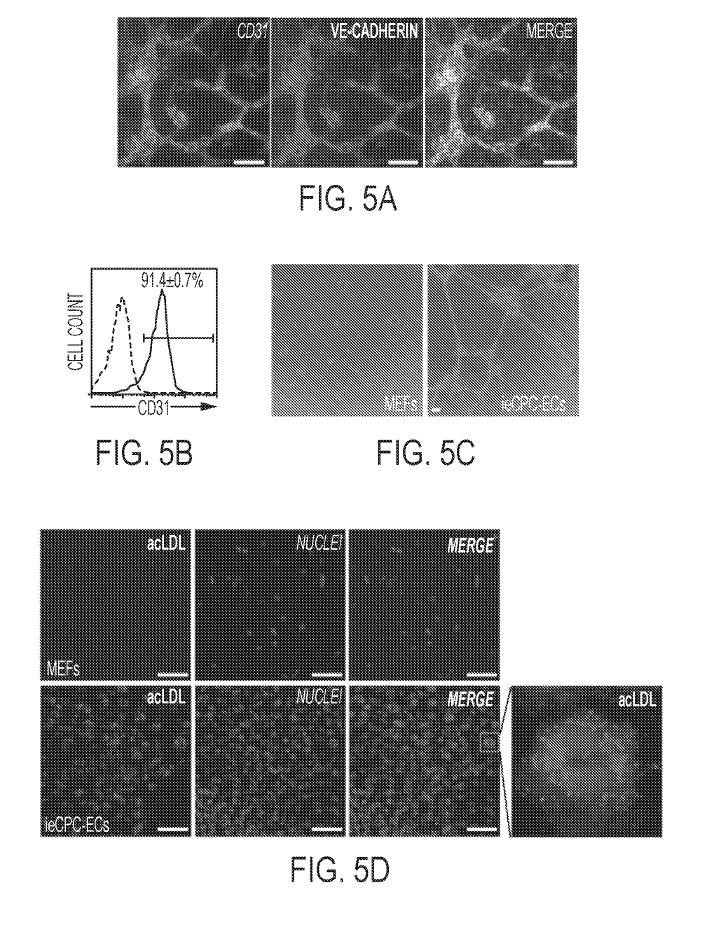

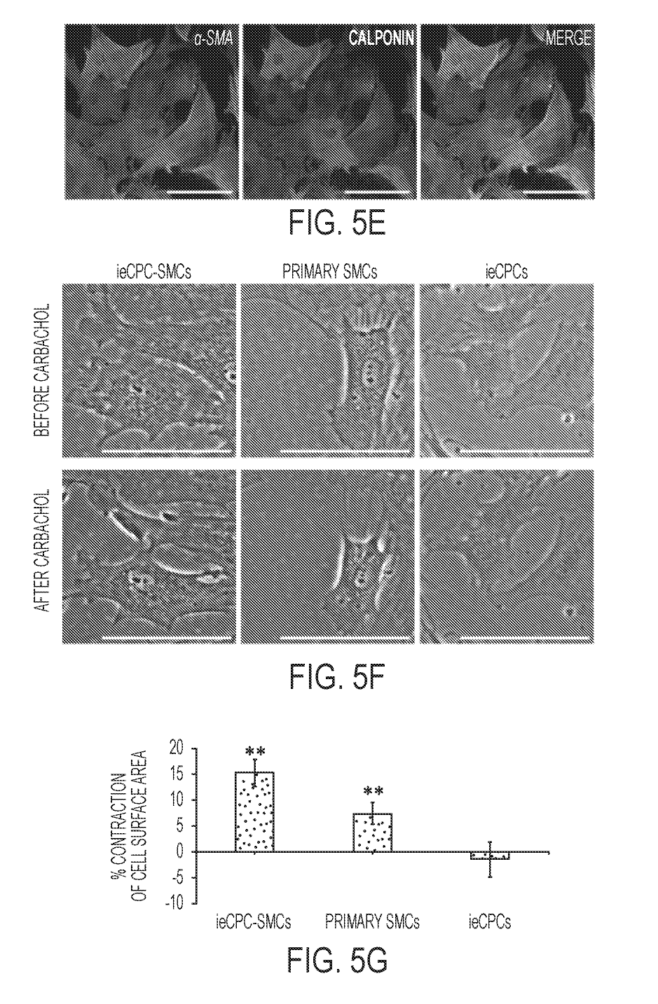

To test whether ieCPCs expanded long-term can give rise to functional endothelial cells (ECs) in vitro, EC gene expression was examined after 10 days of EC differentiation. ECs generated from ieCPCs (ieCPC-ECs) showed typical morphology and highly expressed the EC-specific markers CD31 and VE-cadherin (FIG. 5A). FACS analysis revealed that more than 90% of the total cell population expressed CD31 (FIG. 5B). Moreover, in contrast to the parental MEFs, ieCPC-ECs could robustly form vessel-like structures (FIG. 5C) and efficiently took up fluorescent-labeled acetylated low-density lipoprotein (ac-LDL) (FIG. 5D). These findings confirmed that ieCPC-ECs exhibited a similar phenotype and function to primary ECs (Kaufman et al., Blood 103: 1325-1332 (2004).

To examine SMC differentiation, the expression of SMC-specific markers was analyzed 10 days after SMC differentiation of ieCPCs. Most cells (>98%) were positive for the SMC-specific markers as detected by immunofluorescence staining (FIG. 5E). Carbachol (100 μM) induced contraction of SMCs derived from ieCPCs (ieCPC-SMCs), a phenomenon observed in primary SMCs but not undifferentiated ieCPCs (FIGS. 5F and 5G). These findings show that ieCPC-SMCs have similar functional properties to primary SMCs.

To determine whether ieCPCs are multipotent at a single-cell level, clonal assays were performed on single ieCPCs and the potentials of the ieCPCs were examined to differentiate into CMs, ECs, and SMCs. After 28 days of differentiation, 31.8% of the single ieCPC-derived clones were tripotent, as demonstrated by co-expression of CM, EC, and SMC genes (Table 2). The inventors also found that 22.7% of the clones were bipotent and 45.5% of them were unipotent, in part due to the differentiation conditions and associated efficiency.

TABLE 2
Summary of the cardiovascular-lineage potency of various ieCPC clones

| | | | Single-ieCPC-derived colonies | | | | | | | | | | | | | | | | | | | | | | | |
|---|---|---|---|---|---|---|---|---|---|---|---|---|---|---|---|---|---|---|---|---|---|---|---|---|---|---|
| | | | 1 | 2 | 3 | 4 | 5 | 6 | 7 | 8 | 9 | 10 | 11 | 12 | 13 | 14 | 15 | 16 | 17 | 18 | 19 | 20 | 21 | 22 | Total | % |
| Lineage- | CM | Tnnt2 | + | | + | | + | + | + | | | + | + | + | + | | + | | | + | | + | | | 12 | 54.5 |
| specific | | Actc1 | + | | + | | + | + | + | | | + | + | + | + | | + | | | + | | + | | | | |
| markers | EC | Pecam1 | | | + | | | + | + | | | + | | + | + | | + | | | | | + | | | 8 | 36.4 |
| | | Cdh5 | | | + | | | + | + | | | + | | + | + | | + | | | | | + | | | | |
| | SMC | Myh11 | + | + | + | + | + | + | + | + | + | + | + | + | + | + | | + | + | + | + | + | + | + | 21 | 95.5 |
| | | Tagln | + | + | + | + | + | + | + | + | + | + | + | + | + | + | | + | + | + | + | + | + | + | | |

| | | | | | | | | | | | | | | | | | | | | | | | | | |
|---|---|---|---|---|---|---|---|---|---|---|---|---|---|---|---|---|---|---|---|---|---|---|---|---|---|
| Potency | CM + | | | + | | | + | + | | | + | | + | + | | | | | | | + | | | 7 | 31.8 |
| | EC + | | | | | | | | | | | | | | | | | | | | | | | | |
| | SMC | | | | | | | | | | | | | | | | | | | | | | | | |
| | CM + EC | | | | | | | | | | | | | | | + | | | | | | | | 1 | 4.5 |
| | CM + | + | | | | + | | | | | | + | | | | | | | + | | | | | 4 | 18.2 |
| | SMC | | | | | | | | | | | | | | | | | | | | | | | | |
| | SMC only | | + | | + | | | | + | + | | | | | + | | + | + | | + | | + | + | 10 | 45.5 |

### Example 7: ieCPCs Give Rise to CMs, ECs, and SMCs When Transplanted into Mouse Hearts

After demonstrating that ieCPCs expanded long-term undergo cardiovascular differentiation in vitro, ieCPCs were administered to the native heart environment in vivo.

The ieCPCs were labeled at passage 10 with red fluorescent protein (RFP) and transplanted them into infarcted hearts of immunodeficient mice. Parental MEFs served as a negative control. For each mouse, one million ieCPCs or MEFs were directly injected into the mouse heart immediately after coronary ligation. The recipient mice were sacrificed 2 weeks after transplantation and tissue sections were examined for expression of markers for differentiated cardiovascular lineages.

Control MEFs that were administered did not express CM, EC, and SMC markers and did not convert into cardiovascular cells in the native cardiac in vivo.

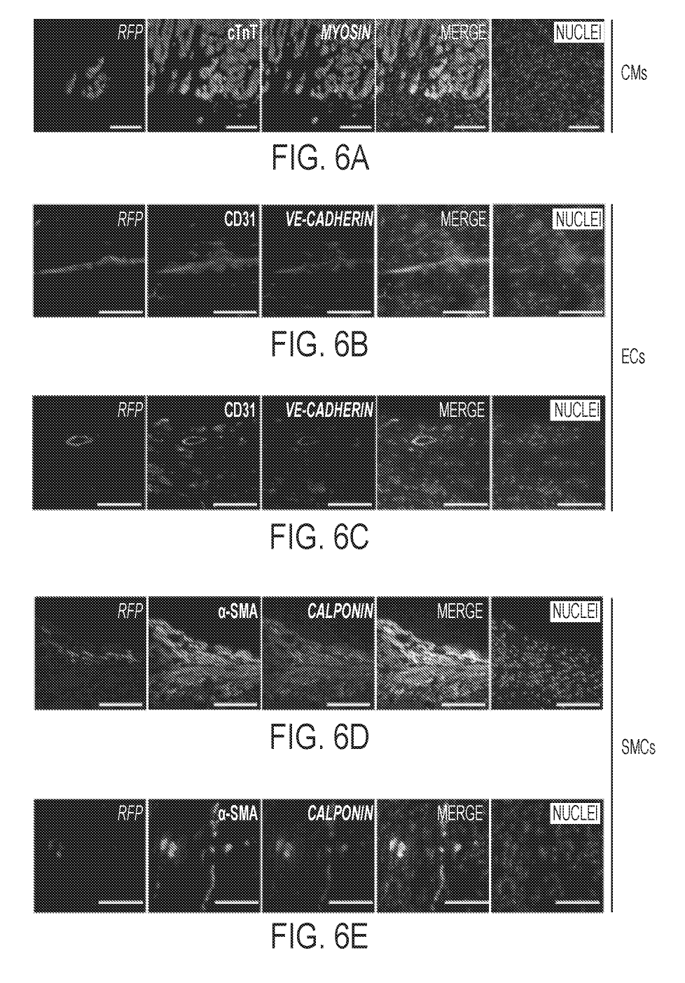

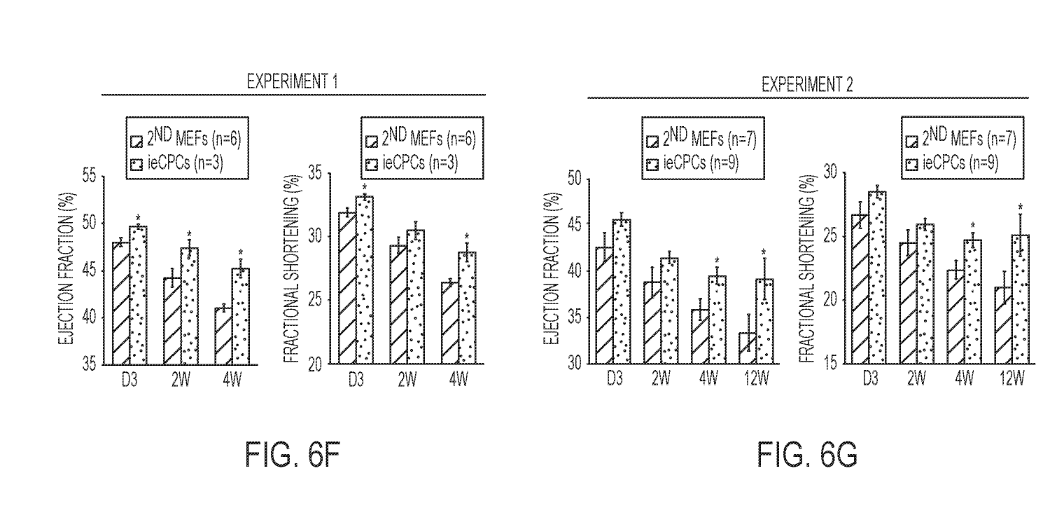

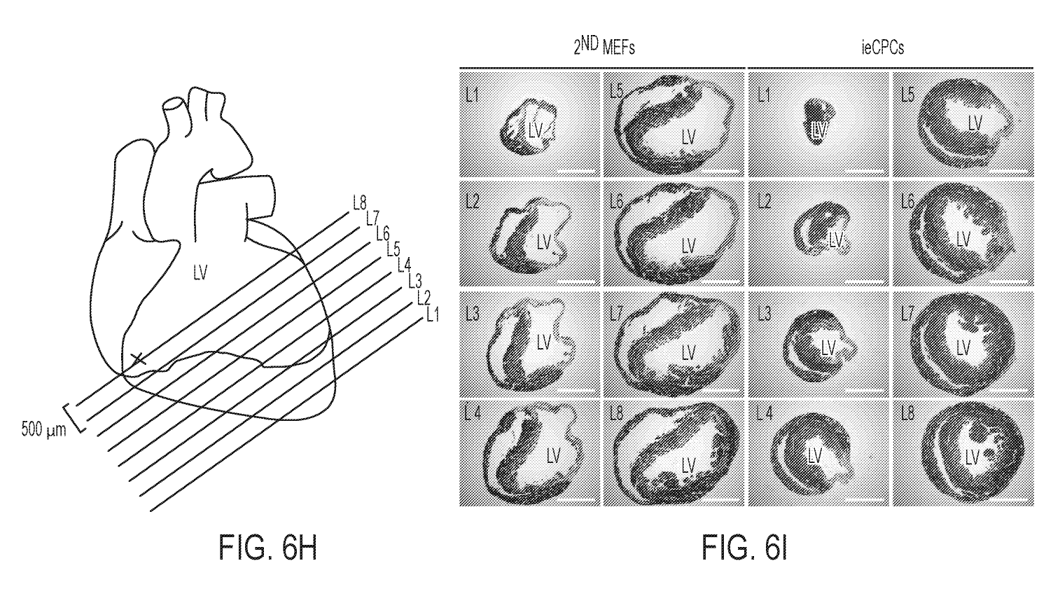

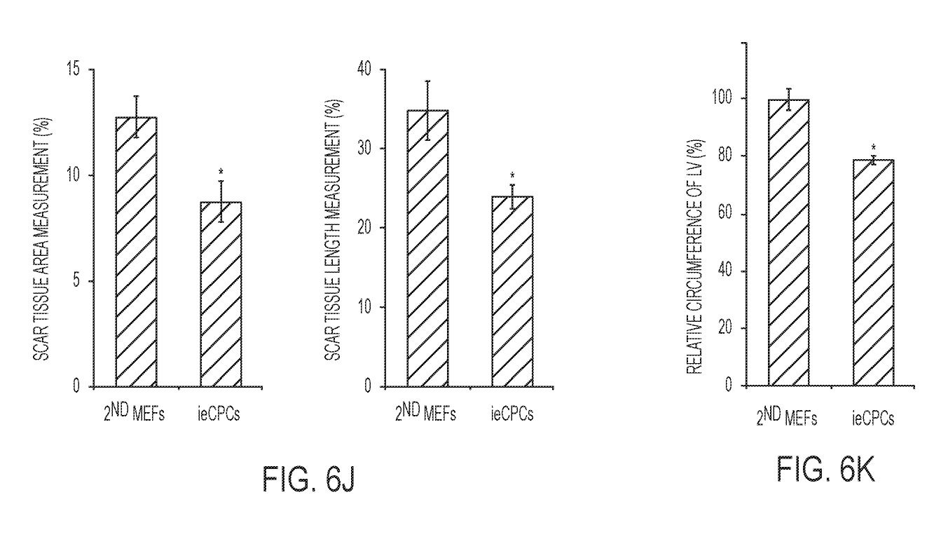

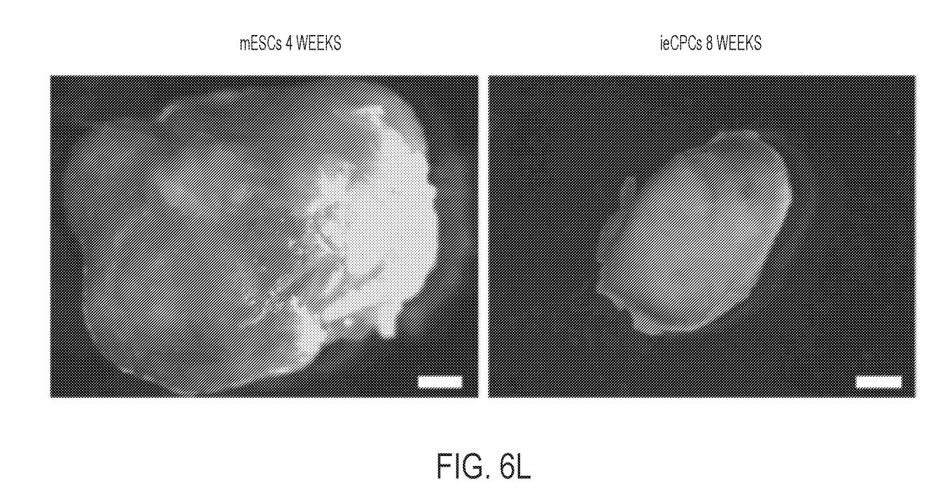

In striking contrast, the detected RFP⁺ ieCPCs co-expressed cTnT and myosin (FIG. 6A), CD31 and VE-cadherin (FIGS. 6B and 6C), or α-SMA and calponin (FIGS. 6D and 6E) in grafts. These data show that ieCPCs successfully converted into CMs, ECs, and SMCs, respectively, in vivo.

RFP⁺ cells were found in capillaries and arterioles that were CD31/VE-cadherin⁺ or α-SMA⁺/calponin⁺, showing that the transplanted cells formed blood vessels, although at a low frequency (FIGS. 6C and 6E). The inventors found that 96 clusters (30.8%) expressed CM marker cTnT, 184 clusters expressed SMC marker α-SMA (59.2%), and 21 clusters expressed EC marker CD31 (6.8%) in 311 engrafted RFP⁺ clusters, showing that more than 90% of the engrafted ieCPCs efficiently differentiated into three cardiovascular cell types after transplantation. Similar to the observations in vitro, the inventors did not detect non-cardiovascular-lineage markers in the grafted ieCPCs.

These results show that the in vivo environment of the infarcted mouse ventricle can trigger multi-lineage cardiovascular differentiation of ieCPCs.

### Example 8: Intramyocardial Transplantation of ieCPCs Retards Adverse Remodeling and Improves Heart Outcome after MI

To determine whether the transplantation of ieCPCs could impact cardiac function following cardiac injury, the inventors examined heart function via high-resolution echocardiography (Echo) over the course of 12 weeks after injecting cells into immunodeficient mice subjected to coronary ligation. All studies were performed in a blinded fashion and were revealed only at the end of the experiments.

The inventors observed that heart function in the control group, injected with parental 2^{nd} MEFs, continued to decline over time, reflected by the left-ventricular fractional shortening and ejection fraction evaluated by Echo (FIGS. 6F and 6G).

In contrast, the inventors discovered that the natural reduction of heart performance post-myocardial infarction was significantly attenuated after transplantation of ieCPCs when compared to the control group, and these differences became more pronounced over time (FIGS. 6F and 6G). Furthermore, significantly smaller scar size was observed in mice transplanted with ieCPCs 12 weeks post-MI (FIGS. 6H-6J). Adverse remodeling, such as dilation and compensatory hypertrophy that are part of the natural course of events after MI, was also reduced in mouse hearts that received ieCPCs (FIGS. 6I and 6K).

Moreover, the inventors did not observe the formation of teratomas in ieCPC-transplanted mice 8 weeks after transplantation (FIG. 6L and Table 3).

TABLE 3
Summary of teratoma-forming ability of mESCs and ieCPCs

| | Injected | Mice bearing | |
|---|---|---|---|
| | Cell Number | teratoma/total | Period |
| Cells | (×10⁶) | mice injected | (weeks) |
| mESCs | 0.5 | 2/2 | 4 |
| ieCPCs | 1 | 0/6 | 8 |

These observations show that transplantation of ieCPCs improves cardiac function after acute ischemic myocardial injury.

### Example 9: BACS Capture and Expand CPCs Derived from PSCs

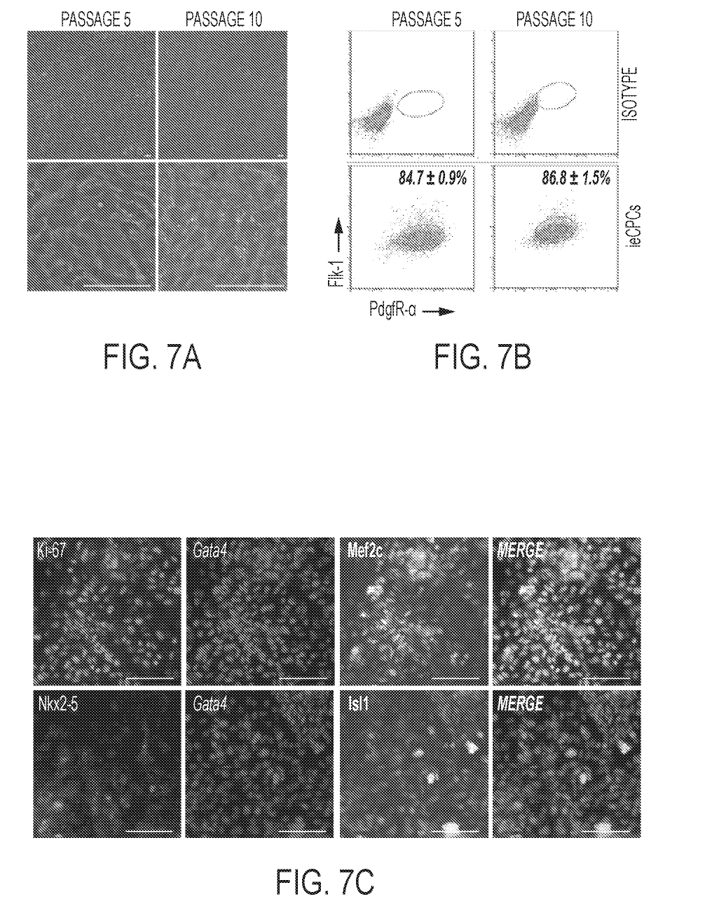

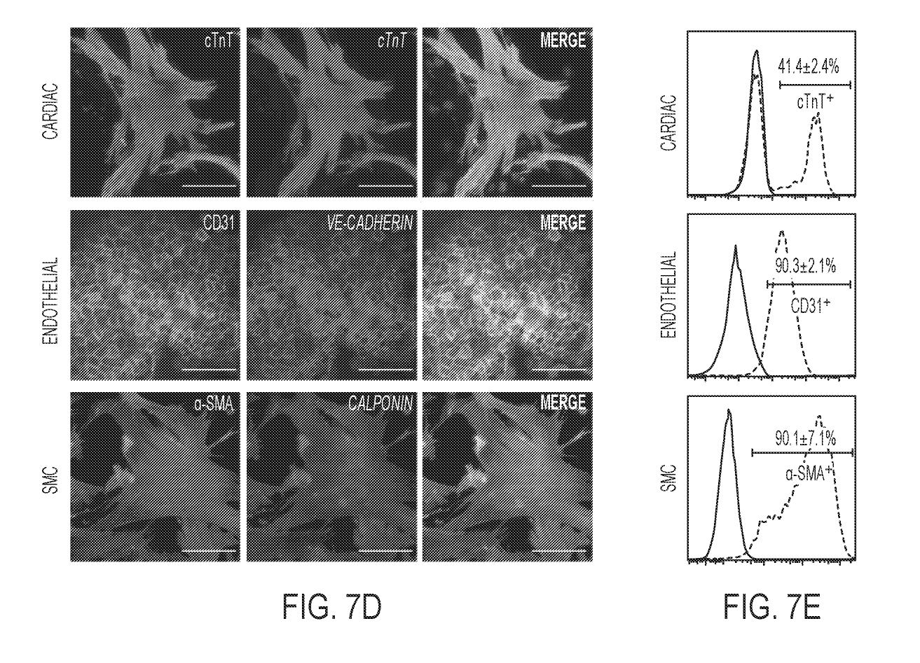

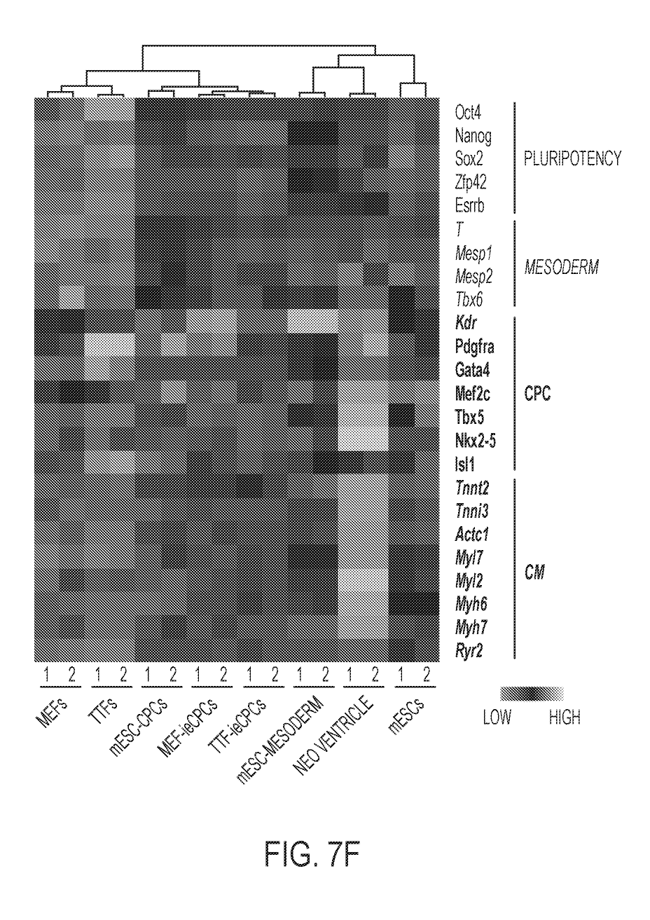

Next, the inventors evaluated whether ieCPCs could be captured during cardiac differentiation of PSCs, which represents embryonic cardiac development. Upon cardiac differentiation of mESCs, Flk-1⁺/PdgfR-α⁺ CPCs were detected at day 3 of differentiation. This population of cells was isolated by FACS and cultured in ieCPC-expansion medium supplemented with BACS. The Flk-1⁺/PdgfR-α⁺ CPCs derived from mESCs exhibited morphologies similar to what was observed for fibroblast-derived ieCPCs (FIG. 7A). The Flk-1⁺/PdgfR-α⁺ CPCs derived from mESCs also expressed CPC and proliferative markers (FIGS. 7B and 7C), robustly expanded in BACS for more than 12 passages, and differentiated into all three cardiovascular lineages with similar efficiencies as fibroblast-derived ieCPCs when stimulated (FIG. 7D-7E). In addition, ieCPCs and mESC-derived CPCs expanded in BACS had very similar gene-expression profiles, exhibiting CPC-specific gene signatures (FIG. 7F). These results demonstrate that ieCPCs appearing during the cardiac transdifferentiation of fibroblasts also exist during normal cardiac differentiation of mESCs.

### Example 10: ieCPCs Can Be Derived from Tail-Tip Fibroblasts

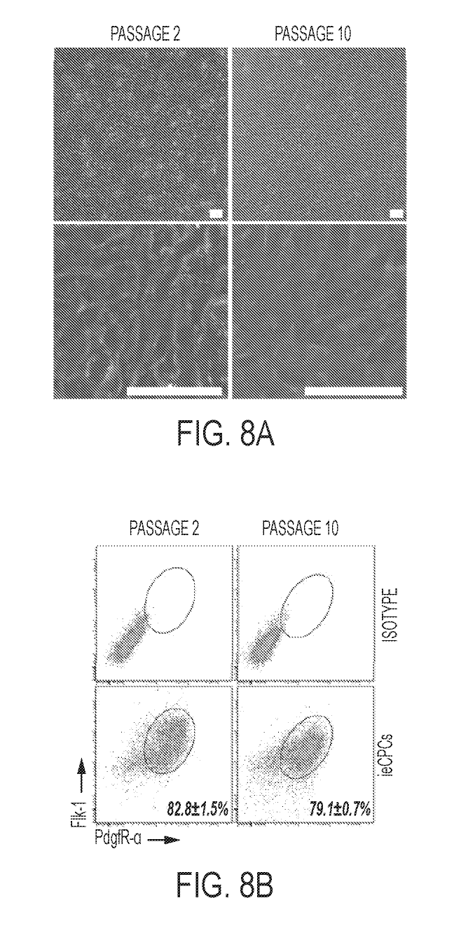

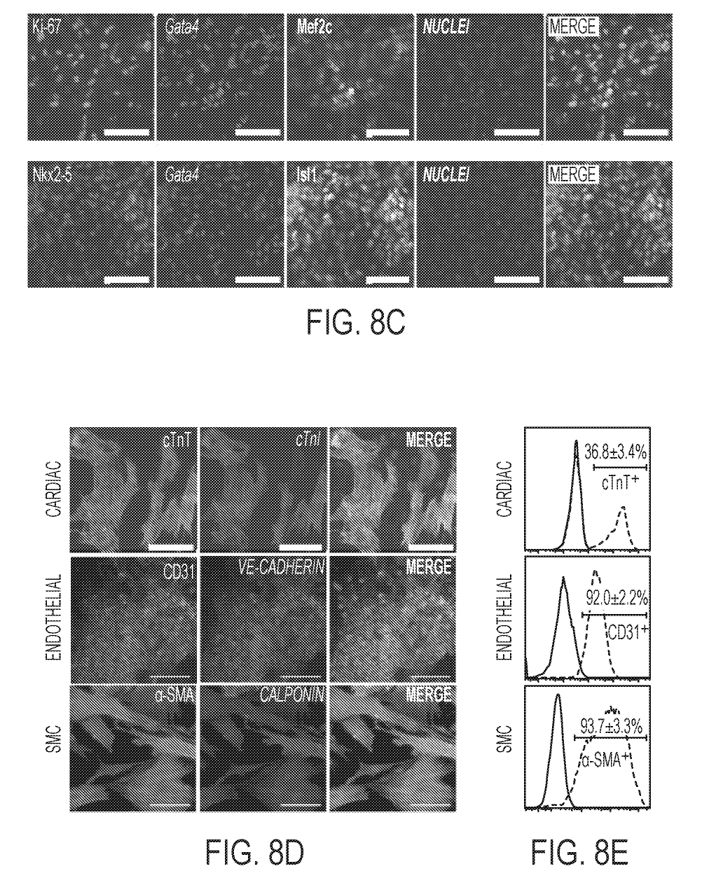

To determine whether ieCPCs could be reprogrammed from other types of fibroblasts that are genetically unmodified, mouse tail-tip fibroblasts (TTFs) were isolated and infected with a lentivirus construct harboring a doxycycline-inducible transgene encoding the reprogramming factors. Under the same conditions as used for 2^{nd} MEFs, a similar Flk-1⁺/PdgfR-α⁺ population was induced from TTFs after BACS treatment. After FACS sorting, these Flk-1⁺/PdgfR-α⁺ cells exhibited normal undifferentiated morphologies (FIG. 8A), sustained expression of CPC and proliferative markers (FIG. 8B-8C), and were steadily expanded in BACS for more than 12 passages.

When cultured in the differentiation conditions of 2^{nd} MEF-derived ieCPCs, the TTF-derived ieCPCs rapidly differentiated into all three cardiovascular lineages with comparable efficiencies (FIG. 8D-8E). These results show that fibroblasts from different tissues of origin can be stably reprogrammed into multipotent ieCPCs, independent of the transgenic system.

### REFERENCES

Birket, M. J., Ribeiro, M. C., Verkerk, A. O., Ward, D., Leitoguinho, A. R., den Hartogh, S. C., Orlova, V. V., Devalla, H. D., Schwach, V., Bellin, M., et al. (2015). Expansion and patterning of cardiovascular progenitors derived from human pluripotent stem cells. Nat Biotechnol.
Blin, G., Nury, D., Stefanovic, S., Neri, T., Guillevic, O., Brinon, B., Bellamy, V., Rucker-Martin, C., Barbry, P., Bel, A., et al. (2010). A purified population of multipotent cardiovascular progenitors derived from primate pluripotent stem cells engrafts in postmyocardial infarcted nonhuman primates. J Clin Invest 120, 1125-1139.
Bruneau, B. G. (2013). Signaling and transcriptional networks in heart development and regeneration. Cold Spring Harb Perspect Biol 5, a008292.
Burridge, P. W., Keller, G., Gold, J. D., and Wu, J. C. (2012). Production of de novo cardiomyocytes: human pluripotent stem cell differentiation and direct reprogramming. Cell Stem Cell 10, 16-28.
Cao, N., Liang, H., Huang, J., Wang, J., Chen, Y., Chen, Z., and Yang, H. T. (2013). Highly efficient induction and long-term maintenance of multipotent cardiovascular progenitors from human pluripotent stem cells under defined conditions. Cell Res 23, 1119-1132.
Chan, S. S., Shi, X., Toyama, A., Arpke, R. W., Dandapat, A., Iacovino, M., Kang, J., Le, G., Hagen, H. R., Garry, D. J., et al. (2013). Mesp1 patterns mesoderm into cardiac, hematopoietic, or skeletal myogenic progenitors in a context-dependent manner. Cell Stem Cell 12, 587-601.
Cheung, C., Bernardo, A. S., Trotter, M. W., Pedersen, R. A., and Sinha, S. (2012). Generation of human vascular smooth muscle subtypes provides insight into embryological origin-dependent disease susceptibility. Nat Biotechnol 30, 165-173.
Devine, W. P., Wythe, J. D., George, M., Koshiba-Takeuchi, K., and Bruneau, B. G. (2014). Early patterning and specification of cardiac progenitors in gastrulating mesoderm. Elife 3.
Efe, J. A., Hilcove, S., Kim, J., Zhou, H., Ouyang, K., Wang, G., Chen, J., and Ding, S. (2011). Conversion of mouse fibroblasts into cardiomyocytes using a direct reprogramming strategy. Nat Cell Biol 13, 215-222.
Hirata, H., Kawamata, S., Murakami, Y., Inoue, K., Nagahashi, A., Tosaka, M., Yoshimura, N., Miyamoto, Y., Iwasaki, H., Asahara, T., et al. (2007). Coexpression of platelet-derived growth factor receptor alpha and fetal liver kinase 1 enhances cardiogenic potential in embryonic stem cell differentiation in vitro. J Biosci Bioeng 103, 412-419.
Kattman, S. J., Witty, A. D., Gagliardi, M., Dubois, N. C., Niapour, M., Hotta, A., Ellis, J., and Keller, G. (2011). Stage-specific optimization of activin/nodal and BMP signaling promotes cardiac differentiation of mouse and human pluripotent stem cell lines. Cell Stem Cell 8, 228-240.
Kaufman, D. S., Lewis, R. L., Hanson, E. T., Auerbach, R., Plendl, J., and Thomson, J. A. (2004). Functional endothelial cells derived from rhesus monkey embryonic stem cells. Blood 103, 1325-1332.
Kuzmenkin, A., Liang, H., Xu, G., Pfannkuche, K., Eichhorn. H., Fatima, A., Luo, H., Saric, T., Wernig, M., Jaenisch, R., et al. (2009). Functional characterization of cardiomyocytes derived from murine induced pluripotent stem cells in vitro. FASEB J 23, 4168-4180.
Lam, J. T., Moretti, A., and Laugwitz, K. L. (2009). Multipotent progenitor cells in regenerative cardiovascular medicine. Pediatr Cardiol 30, 690-698.
Laugwitz, K. L., Moretti, A., Lam, J., Gruber, P., Chen, Y., Woodard, S., Lin, L. Z., Cai, C. L., Lu, M. M., Reth, M., et al. (2005). Postnatal isl1+ cardioblasts enter fully differentiated cardiomyocyte lineages. Nature 433, 647-653.
Moretti, A., Caron, L., Nakano, A., Lam, J. T., Bernshausen, A., Chen, Y., Qyang. Y., Bu, L., Sasaki, M., Martin-Puig, S., et al. (2006). Multipotent embryonic isl1+ progenitor cells lead to cardiac, smooth muscle, and endothelial cell diversification. Cell 127, 1151-1165.
Murry, C. E., and Keller, G. (2008). Differentiation of embryonic stem cells to clinically relevant populations: lessons from embryonic development. Cell 132, 661-680.
Naito, A. T., Shiojima, I., Akazawa, H., Hidaka, K., Morisaki, T., Kikuchi, A., and Komuro, I. (2006). Developmental stage-specific biphasic roles of Wnt/beta-catenin signaling in cardiomyogenesis and hematopoiesis. Proc Natl Acad Sci USA 103, 19812-19817.
Qyang, Y., Martin-Puig, S., Chiravuri, M., Chen, S., Xu, H., Bu, L., Jiang, X., Lin, L., Granger, A., Moretti, A., et al. (2007). The renewal and differentiation of Isl1+ cardiovascular progenitors are controlled by a Wnt/beta-catenin pathway. Cell Stem Cell 1, 165-179.
van Laake, L. W., Passier, R., Monshouwer-Kloots, J., Verkleij, A. J., Lips, D. J., Freund, C., den Ouden, K., Ward-van Oostwaard, D., Korving, J., Tertoolen, L. G., et al. (2007). Human embryonic stem cell-derived cardiomyocytes survive and mature in the mouse heart and transiently improve function after myocardial infarction. Stem Cell Res 1, 9-24.
Wamstad, J. A., Alexander, J. M., Truty, R. M., Shrikumar, A., Li, F., Eilertson, K. E., Ding, H., Wylie, J. N., Pico, A. R., Capra, J. A., et al. (2012). Dynamic and coordinated epigenetic regulation of developmental transitions in the cardiac lineage. Cell 151, 206-220.
Wang, H., Cao, N., Spencer, C. I., Nie, B., Ma, T., Xu, T., Zhang, Y., Wang, X., Srivastava, D., and Ding, S. (2014). Small molecules enable cardiac reprogramming of mouse fibroblasts with a single factor, Oct4. Cell Rep 6, 951-960.
Yang, L., Soonpaa, M. H., Adler, E. D., Roepke, T. K., Kattman, S. J., Kennedy, M., Henckaerts, E., Bonham, K., Abbott, G. W., Linden, R. M., et al. (2008). Human cardiovascular progenitor cells develop from a KDR+ embryonic-stem-cell-derived population. Nature 453, 524-528.
Carey, B. W., Markoulaki, S., Hanna, J., Saha, K., Gao, Q., Mitalipova, M., and Jaenisch, R. (2009). Reprogramming of murine and human somatic cells using a single polycistronic vector. Proceedings of the National Academy of Sciences of the United States of America 106, 157-162.
Devine, W. P., Wythe, J. D., George, M., Koshiba-Takeuchi, K., and Bruneau, B. G. (2014). Early patterning and specification of cardiac progenitors in gastrulating mesoderm. eLife 3.
Fu, J. D., Stone, N. R., Liu, L., Spencer, C. I., Qian, L., Hayashi, Y., Delgado-Olguin, P., Ding, S., Bruneau, B. G., and Srivastava, D. (2013). Direct reprogramming of human fibroblasts toward a cardiomyocyte-like state. Stem cell reports 1, 235-247.
Grigoriadis, A. E., Kennedy, M., Bozec, A., Brunton, F., Stenbeck, G., Park, I. H., Wagner, E. F., and Keller, G. M. (2010). Directed differentiation of hematopoietic precursors and functional osteoclasts from human ES and iPS cells. Blood 115, 2769-2776.
Kim, D., Pertea, G., Trapnell, C., Pimentel, H., Kelley, R., and Salzberg, S. L. (2013). TopHat2: accurate alignment of transcriptomes in the presence of insertions, deletions and gene fusions. Genome biology 14, R36.
Lee, R. H., Kim, B., Choi, I., Kim, H., Choi, H. S., Suh, K., Bae, Y. C., and Jung, J. S. (2004). Characterization and expression analysis of mesenchymal stem cells from human bone marrow and adipose tissue. Cellular physiology and biochemistry: international journal of experimental cellular physiology, biochemistry, and pharmacology 14, 311-324.
Liao, Y., Smyth, G. K., and Shi, W. (2014). featureCounts: an efficient general purpose program for assigning sequence reads to genomic features. Bioinformatics 30, 923-930.
Mizuno, Y., Chang, H., Umeda, K., Niwa, A., Iwasa, T., Awaya, T., Fukada, S., Yamamoto, H., Yamanaka, S., Nakahata, T., et al. (2010). Generation of skeletal muscle stem/progenitor cells from murine induced pluripotent stem cells. FASEB journal: official publication of the Federation of American Societies for Experimental Biology 24, 2245-2253.
Qian, L., Huang, Y., Spencer, C. I., Foley, A., Vedantham, V., Liu, L., Conway, S. J., Fu, J. D., and Srivastava, D. (2012). In vivo reprogramming of murine cardiac fibroblasts into induced cardiomyocytes. Nature 485, 593-598.
Risso, D., Ngai, J., Speed, T. P., and Dudoit, S. (2014). Normalization of RNA-seq data using factor analysis of control genes or samples. Nature biotechnology 32, 896-902.
Robinson, M. D., McCarthy, D. J., and Smyth, G. K. (2010). edgeR: a Bioconductor package for differential expression analysis of digital gene expression data. Bioinformatics 26, 139-140.
Takagawa, J., Zhang, Y., Wong, M. L., Sievers, R. E., Kapasi, N. K., Wang, Y., Yeghiazarians, Y., Lee, R. J., Grossman, W., and Springer, M. L. (2007). Myocardial infarct size measurement in the mouse chronic infarction model: comparison of area- and length-based approaches. Journal of applied physiology 102, 2104-2111.

Wamstad, J. A., Alexander, J. M., Truty, R. M., Shrikumar, A., Li, F., Eilertson, K. E., Ding, H., Wylie, J. N., Pico, A. R., Capra, J. A., et al. (2012). Dynamic and coordinated epigenetic regulation of developmental transitions in the cardiac lineage. Cell 151, 206-220.
Wernig, M., Lengner, C. J., Hanna, J., Lodato, M. A., Steine, E., Foreman, R., Staerk, J., Markoulaki, S., and Jaenisch, R. (2008). A drug-inducible transgenic system for direct reprogramming of multiple somatic cell types. Nat Biotechnol 26, 916-924.
Wobus, A. M., Guan, K., Yang, H. T., and Boheler, K. R. (2002). Embryonic stem cells as a model to study cardiac, skeletal muscle, and vascular smooth muscle cell differentiation. Methods in molecular biology 185, 127-156.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A method for expanding cardiovascular progenitor cells comprising contacting the cardiovascular progenitor cells with a culture medium comprising BMP4, Activin A, a glycogen synthase kinase 3 inhibitor, and an inhibitor of FGF, VEGF, and PDGF signaling, to generate an expanded cardiovascular progenitor cell population.
2. The method of statement 1, wherein the glycogen synthase kinase 3 inhibitor is CHIR99021, 1-azakenpaullone, AR-A014418, indirubin-3′-monoxime, 5-Iodo-indirubin-3′-monoxime, kenpaullone, SB-415286, SB-216763, 2-anilino-5-phenyl-1,3,4-oxadiazole), (Z)-5-(2,3-Memylenedioxyphenyl)imidazolidine-2,4-dione, TWS119, CHIR98014, SB415286, Tideglusib, LY2090314, a lithium salt, or a combination thereof.
3. The method of statement 1 or 2, wherein the glycogen synthase kinase 3 inhibitor is CHIR99021.
4. The method of any of statements 1-3, wherein the inhibitor of FGF, VEGF, and PDGF signaling is SU5402, AP 24534, FIN 1 hydrochloride, R 1530, SU 6668, Sunitinib malate, Toceranib, Brivanib alaninate, or a combination thereof.
5. The method of any of statements 1-4, wherein the inhibitor of FGF, VEGF, and PDGF signaling is SU5402.
6. The method of any of statements 1-5, wherein the BMP4 is present in the culture medium at a concentration of 0.5 to 50 ng/mL, about 0.5 to 20 ng/ml, about 1.0 to 30 ng/mL, or 1 to 15 ng/ml, about 1.5 to 20 ng/mL, 2 to 10 ng/ml, about 2.0 to 15 ng/mL, about 2.5 to 10 ng/mL, about 3 to 7 ng/ml, about 3 to 8 ng/m L, or 4 to 6 ng/ml, or about 4 to 6 ng/m L.
7. The method of any of statements 1-6, wherein the Activin A is present in the culture medium at a concentration of about 0.5 ng/ml to about 100 ng/ml, or about 1.0 ng/ml to about 75 ng/ml, or from about 1 to 30 ng/ml, or from about 2 to 25 ng/ml, or from about 2 ng/ml to about 50 ng/ml, or from about 3 to 20 ng/ml, or from about 3 ng/ml to about 40 ng/ml, or from about or 5 to 15 ng/ml, or from about 5 ng/ml to about 30 ng/ml, or from about 6 ng/ml to about 20 ng/ml, or from about 7 ng/ml to about 15 ng/ml, or from about 7 to 13 ng/ml, or from about 8 ng/ml to about 12 ng/ml, or about 10 ng/ml.
8. The method of any of statements 1-7, wherein the glycogen synthase kinase 3 inhibitor is present in the culture medium at a concentration of about 0.01 micromolar to about 1 millimolar in a solution, or about 0.1 micromolar to about 100 micromolar in a solution, or about 0.5 micromolar to about 10 micromolar in a solution, or about 1 micromolar to about 8 micromolar in a solution, or about 1.5 micromolar to about 7 micromolar in a solution, or about 2 micromolar to about 5 micromolar in a solution, or at about 3 micromolar.
9. The method of any of statements 1-8, wherein the inhibitor of FGF, VEGF, and PDGF signaling is present in the culture medium at a concentration of about 0.01 micromolar to about 1 millimolar in a solution, or about 0.1 micromolar to about 100 micromolar in a solution, or about 0.5 micromolar to about 10 micromolar in a solution, or about 1 micromolar to about 5 micromolar in a solution, or at a concentration of about 2 micromolar.
10. The method of any of statements 1-9, wherein the culture medium also comprises Jak inhibitor 1, ascorbic acid, or a combination thereof.
11. The method of any of statements 1-10, wherein the cardiovascular progenitor cells can be sub-cultured at least 3 times, or at least 5 times, or at least 9 times, or at least 12 times, or at least 15 times, or at least 18 times, or at least 20 times, or at least 25 times without loss of phenotype or genotype.
12. The method of any of statements 1-11, wherein the cardiovascular progenitor cells express Gata4, Mef2c, Tbx5, and Nkx2-5 before contact with the culture medium.
13. The method of any of statements 1-12, wherein the cardiovascular progenitor cells express Flk1 and PdgJra before contact with the culture medium.
14. The method of any of statements 1-13, wherein cells in the expanded cardiovascular progenitor cell population express Gata4. Mef2c, Tbx5, and Nkx2-5 after contact with the culture medium.
15. The method of any of statements 1-14, wherein cells in the expanded cardiovascular progenitor cell population express Flk1 and Pdgfra after contact with the culture medium.
16. The method of any of statements 1-15, wherein the cardiovascular progenitor cells express little or no differentiated CM markers before contact with the culture medium.
17. The method of any of statements 1-16, wherein the cardiovascular progenitor cells do not express cardiomyocyte markers selected from Actc1, Tnnt2, cTnI, cTnT, alpha-actinin, myosin, or any combination thereof before contact with the culture medium.
18. The method of any of statements 1-17, wherein the expanded cardiovascular progenitor cells do not express cardiomyocyte markers selected from Actc1, Tnnt2, cTnI, cTnT, alpha-actinin, myosin, or any combination thereof after contact with the culture medium.
19. The method of any of statements 1-18, wherein the cardiovascular progenitor cells expand by at least 100-fold, or at least 1000-fold, or at least 10,000-fold, or at least 100,000-fold, or at least 1,000,000-fold, or at least 10,000,000-fold, or at least 100,000,000-fold, or at least 1,000,000,000-fold.
20. The method of any of statements 1-19, wherein the cardiovascular progenitor cells express little or no Tnnt2 and Myh6, before and after contact with the culture medium so long as the cardiovascular progenitor cells are not contacted with factors that reduce or increase differentiation.
21. The method of any of statements 1-20, further comprising differentiating the cardiovascular progenitor cells with one or more factors that increase differentiation.
22. The method of any of statements 1-21, wherein the one or more factors that increase differentiation are selected from serum-free differentiation (SFD) medium, N2 supplement, B27 without Vitamin A, B27 with vitamin A, Glutamax, BSA, monothioglycerol (MTG), ascorbic acid, IWP2, VEGF, TGF-β1, PDGF-BB, FBS, or any combination thereof.
23. The method of any of statements 1-22, further comprising differentiating the cardiovascular progenitor cells into cardiomyocytes (CMs), endothelial cells (ECs), smooth muscle cell (SMC), or combinations thereof.
24. The method of any of statements 1-23, further comprising differentiating the expanded cardiovascular progenitor cells into cardiomyocytes (CMs) by culturing the cardiovascular progenitor cells on matrigel-coated plates at a density of about 1×10⁴ cells/cm² to about 1×10⁶, or about 3×10⁵ cells/cm² in serum-free differentiation (SFD) medium with IWP2.
25. The method of any of statements 1-24, further comprising differentiating the expanded cardiovascular progenitor cells into cardiomyocytes (CMs) by culturing the cardiovascular progenitor cells on matrigel-coated plates at a density of about 1×10⁴ cells/cm² to about 1×10⁶, or about 3×10⁵ cells/cm² in serum-free differentiation (SFD) medium with IWP2 for 10 days.
26. The method of any of statements 1-25, further comprising differentiating the expanded cardiovascular progenitor cells into cardiomyocytes (CMs) by culturing the cardiovascular progenitor cells on matrigel-coated plates at a density of about 1×10⁴ cells/cm² to about 1×10⁶, or about 3×10′ cells/cm² in serum-free differentiation (SFD) medium with IWP2, where the IWP2 is at a concentration of about 0.01 micromolar to about 1 millimolar in a solution, or about 0.1 micromolar to about 100 micromolar in a solution, or about 0.5 micromolar to about 10 micromolar in a solution, or about 1 micromolar to about 9 micromolar in a solution, or about 2 micromolar to about 8 micromolar in a solution, or about 3 micromolar to about 7 micromolar in a solution, or at about 5 micromolar.
27. The method of any of statements 1-23, further comprising differentiating the expanded cardiovascular progenitor cells into smooth muscle cells (SMCs).
28. The method of any of statements 1-23, or 27, where the expanded cardiovascular progenitor cells are differentiated into smooth muscle cells (SMCs) in a culture medium supplemented with TGF-β1 and PDGF-BB.
29. The method of any of statements 1-23, 27, or 28, where the expanded cardiovascular progenitor cells are differentiated into smooth muscle cells (SMCs) in a culture medium supplemented with TGF-β1 and PDGF-BB for about 7 to about 14 days.
30. The method of any of statements 1-23, 27, 28, or 29, where the expanded cardiovascular progenitor cells are differentiated into smooth muscle cells (SMCs) in a culture medium supplemented with TGF-β1 (2 ng/ml) and PDGF-BB (10 ng/ml).
31. The method of any of statements 1-30, further comprising administering the expanded cardiovascular progenitor cells, the cardiomyocytes, the smooth muscle cells, or any combination thereof to a mammal.
32. The method of any of statements 1-31, further comprising administering the expanded cardiovascular progenitor cells, the cardiomyocytes, the smooth muscle cells, or any combination thereof to a mammal in need thereof.
33. The method of statement 31 or 32, wherein the mammal has a cardiac disease or condition.
34. The method of any of statements 31-33, wherein the mammal suffers from a cardiac disease or condition congestive heart failure, myocardial infarction, cardiac ischemia, myocarditis, arrhythmia or any combination thereof.
35. The method of any of statements 31-34, wherein the mammal is a human.
36. A composition comprising BMP4, Activin A, a glycogen synthase kinase 3 inhibitor, and an inhibitor of FGF, VEGF, and PDGF signaling.
37. The composition of statement 36, wherein the glycogen synthase kinase 3 inhibitor is CHIR99021, 1-azakenpaullone, AR-A014418, indirubin-3′-monoxime, 5-Iodo-indirubin-3′-monoxime, kenpaullone, SB-415286, SB-216763, 2-anilino-5-phenyl-1,3,4-oxadiazole), (Z)-5-(2,3-Memylenedioxyphenyl)imidazolidine-2,4-dione, TWS119, CHIR98014, SB415286, Tideglusib, LY2090314, a lithium salt, or a combination thereof.
38. The composition of statement 36 or 37, wherein the glycogen synthase kinase 3 inhibitor is CHIR99021.
39. The composition of any of statements 36-38, wherein the inhibitor of FGF, VEGF, and PDGF signaling is SU5402, AP 24534, FIN 1 hydrochloride, R 1530, SU 6668, Sunitinib malate, Toceranib; Brivanib alaninate, or a combination thereof.
40. The composition of any of statements 36-39, wherein the inhibitor of FGF, VEGF, and PDGF signaling is SU5402.
41. The composition of any of statements 36-40, wherein the BMP4 is present in the culture medium at a concentration of 0.5 to 50 ng/mL, about 0.5 to 20 ng/ml, about 1.0 to 30 ng/mL, or 1 to 15 ng/ml, about 1.5 to 20 ng/mL, 2 to 10 ng/ml, about 2.0 to 15 ng/mL, about 2.5 to 10 ng/mL, about 3 to 7 ng/ml, about 3 to 8 ng/mL, or 4 to 6 ng/ml, or about 4 to 6 ng/mL.
42. The composition of any of statements 36-41, wherein the Activin A is present in the culture medium at a concentration of about 0.5 ng/ml to about 100 ng/ml, or about 1.0 ng/ml to about 75 ng/ml, or from about 1 to 30 ng/ml, or from about 2 to 25 ng/ml, or from about 2 ng/ml to about 50 ng/ml, or from about 3 to 20 ng/ml, or from about 3 ng/ml to about 40 ng/ml, or from about or 5 to 15 ng/ml, or from about 5 ng/ml to about 30 ng/ml, or from about 6 ng/ml to about 20 ng/ml, or from about 7 ng/ml to about 15 ng/ml, or from about 7 to 13 ng/ml, or from about 8 ng/ml to about 12 ng/ml, or about 10 ng/ml.
43. The composition of any of statements 36-42, wherein the glycogen synthase kinase 3 inhibitor is present in the culture medium at a concentration of about 0.01 micromolar to about 1 millimolar in a solution, or about 0.1 micromolar to about 100 micromolar in a solution, or about 0.5 micromolar to about 10 micromolar in a solution, or about 1 micromolar to about 8 micromolar in a solution, or about 1.5 micromolar to about 7 micromolar in a solution, or about 2 micromolar to about 5 micromolar in a solution, or at about 3 micromolar.
44. The composition of any of statements 36-43, wherein the inhibitor of FGF, VEGF, and PDGF signaling is present in the culture medium at a concentration of about 0.01 micromolar to about 1 millimolar in a solution, or about 0.1 micromolar to about 100 micromolar in a solution, or about 0.5 micromolar to about 10 micromolar in a solution, or about 1 micromolar to about 5 micromolar in a solution, or at a concentration of about 2 micromolar.
45. The composition of any of statements 36-44, wherein the culture medium also comprises Jak inhibitor 1, ascorbic acid, or a combination thereof.
46. The composition of any of statements 36-44, formulated as a cell culture medium.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential.

The methods and processes illustratively described herein suitably may be practiced in differing orders of steps, and the methods and processes are not necessarily restricted to the orders of steps indicated herein or in the claims. As used herein and in the appended claims, the singular forms “a,” “an,” and “the” include plural reference unless the context clearly dictates otherwise. Thus, for example, a reference to “a nucleic acid” or “a promoter” includes a plurality of such nucleic acids or promoters (for example, a solution of nucleic acids or a series of promoters), and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as summarized by the statements of the invention and as defined by the appended claims.

What is claimed:

1. A method for expanding cardiovascular progenitor cells comprising contacting the cardiovascular progenitor cells with a culture medium comprising BMP4, Activin A, a glycogen synthase kinase 3 inhibitor, and an inhibitor of FGF, VEGF, and PDGF signaling, to generate an expanded cardiovascular progenitor cell population.

2. The method of claim 1, wherein the glycogen synthase kinase 3 inhibitor is CHIR99021, 1-azakenpaullone, AR-A014418, indirubin-3′-monoxime, 5-Iodo-indirubin-3′-monoxime, kenpaullone, SB-415286, SB-216763, 2-anilino-5-phenyl-1,3,4-oxadiazole), (Z)-5-(2,3-Memylenedioxyphenyl)imidazolidine-2,4-dione, TWS119, CHIR98014, SB415286, Tideglusib, LY2090314, a lithium salt, or a combination thereof.

3. The method of claim 1, wherein the inhibitor of FGF, VEGF, and PDGF signaling is SU5402, AP 24534, FIIN 1 hydrochloride, R 1530, SU 6668, Sunitinib malate, Toceranib; Brivanib alaninate, or a combination thereof.

4. The method of claim 1, wherein the BMP4 is present in the culture medium at a concentration of 0.5 to 50 ng/mL, about 0.5 to 20 ng/ml.

5. The method of claim 1, wherein the Activin A is present in the culture medium at a concentration of about 0.5 ng/ml to about 100 ng/ml.

6. The method of claim 1, wherein the glycogen synthase kinase 3 inhibitor is present in the culture medium at a concentration of about 0.01 micromolar to about 1 millimolar.

7. The method of claim 1, wherein the inhibitor of FGF, VEGF, and PDGF signaling is present in the culture medium at a concentration of about 0.01 micromolar to about 1 millimolar.

8. The method claim 1, wherein the culture medium also comprises Jak inhibitor 1, ascorbic acid, or a combination thereof.

9. The method of claim 1, wherein the cardiovascular progenitor cells can be sub-cultured at least 3 times without loss of phenotype or genotype.

10. The method of claim 1, wherein the cardiovascular progenitor cells express Gata4, Mef2c, Tbx5, and Nkx2-5 before and after contact with the culture medium.

11. The method of claim 1, wherein the cardiovascular progenitor cells express Flk1 and Pdgfra before and after contact with the culture medium.

12. The method of claim 1, wherein the cardiovascular progenitor cells express little or no differentiated cardiomyocyte markers before or after contact with the culture medium.

13. The method of claim 1, wherein the cardiovascular progenitor cells expand at least 100-fold.

14. The method of claim 1, wherein the cardiovascular progenitor cells express little or no Tnnt2 and Myh6, before and after contact with the culture medium.

15. The method of claim 1, further comprising differentiating the cardiovascular progenitor cells into cardiomyocytes (CMs), endothelial cells (ECs), smooth muscle cell (SMC), or combinations thereof.

16. The method of claim 1, further comprising differentiating the expanded cardiovascular progenitor cells into cardiomyocytes (CMs) by culturing the cardiovascular progenitor cells in serum-free differentiation (SFD) medium with IWP2.

17. The method of claim 1, further comprising differentiating the expanded cardiovascular progenitor cells into smooth muscle cells (SMCs).

18. The method of claim 1, further comprising administering the expanded cardiovascular progenitor cells, or cardiomyocytes differentiated therefrom, or smooth muscle cells derived therefrom, or any combination thereof to a mammal.

19. The method of claim 18, wherein the mammal has a cardiac disease or condition.

20. A composition comprising BMP4, Activin A, a glycogen synthase kinase 3 inhibitor, and an inhibitor of FGF, VEGF, and PDGF signaling.

21. The composition of claim 20, wherein the glycogen synthase kinase 3 inhibitor is CHIR99021, 1-azakenpaullone, AR-A014418, indirubin-3′-monoxime, 5-Iodo-indirubin-3′-monoxime, kenpaullone, SB-415286, SB-216763, 2-anilino-5-phenyl-1,3,4-oxadiazole), (Z)-5-(2,3-Memylenedioxyphenyl)imidazolidine-2,4-dione, TWS119, CHIR98014, SB415286, Tideglusib, LY2090314, a lithium salt, or a combination thereof.

22. The composition of claim 20, wherein the inhibitor of FGF, VEGF, and PDGF signaling is SU5402, AP 24534, FIN 1 hydrochloride, R 1530, SU 6668, Sunitinib malate, Toceranib, Brivanib alaninate, or a combination thereof.

23. The composition of claim 20, wherein the culture medium also comprises Jak inhibitor 1, ascorbic acid, or a combination thereof.

24. The composition of claim 20, formulated as a cell culture medium.


SEQUENCE LISTING (100 sequences)

SEQ ID NO: 1 - DNA, 23 bp, Artificial Sequence
  A synthetic oligonucleotide
ttgaactttcctccatgtgctga

SEQ ID NO: 2 - DNA, 20 bp, Artificial Sequence
  A synthetic oligonucleotide
tcccaagagcctgccacttt

SEQ ID NO: 3 - DNA, 20 bp, Artificial Sequence
  A synthetic oligonucleotide
catcgttccagtacgcagaa

SEQ ID NO: 4 - DNA, 20 bp, Artificial Sequence
  A synthetic oligonucleotide
ctagaagagccagcatgtcg

SEQ ID NO: 5 - DNA, 20 bp, Artificial Sequence
  A synthetic oligonucleotide
cccagagcctaggaacaaga

SEQ ID NO: 6 - DNA, 21 bp, Artificial Sequence
  A synthetic oligonucleotide
gggttctggagacacagaaag

SEQ ID NO: 7 - DNA, 20 bp, Artificial Sequence
  A synthetic oligonucleotide
ccgagaaaatggcagaaact

SEQ ID NO: 8 - DNA, 20 bp, Artificial Sequence
  A synthetic oligonucleotide
gtgtatccccactcccacag

SEQ ID NO: 9 - DNA, 20 bp, Artificial Sequence
  A synthetic oligonucleotide
aaacctcctgcaagcaaatg

SEQ ID NO: 10 - DNA, 20 bp, Artificial Sequence
  A synthetic oligonucleotide
tccagaatcctcttccatgc

SEQ ID NO: 11 - DNA, 20 bp, Artificial Sequence
  A synthetic oligonucleotide
agaggaggagcttgagggag

SEQ ID NO: 12 - DNA, 20 bp, Artificial Sequence
  A synthetic oligonucleotide
agaaaatccgatacccggag

SEQ ID NO: 13 - DNA, 20 bp, Artificial Sequence
  A synthetic oligonucleotide
ctggaagacaccccaatctc

SEQ ID NO: 14 - DNA, 20 bp, Artificial Sequence
  A synthetic oligonucleotide
caggcattgcacaggtagtg

SEQ ID NO: 15 - DNA, 20 bp, Artificial Sequence
  A synthetic oligonucleotide
tggagagatgaagtgaagcg

SEQ ID NO: 16 - DNA, 20 bp, Artificial Sequence
  A synthetic oligonucleotide
gcacagctcagttcccaaat

SEQ ID NO: 17 - DNA, 20 bp, Artificial Sequence
  A synthetic oligonucleotide
ggcagtgatgacctggagtt

SEQ ID NO: 18 - DNA, 20 bp, Artificial Sequence
  A synthetic oligonucleotide
tggttggaggtgactttgtg

SEQ ID NO: 19 - DNA, 20 bp, Artificial Sequence
  A synthetic oligonucleotide
ccaagtgctctcctgctttc

SEQ ID NO: 20 - DNA, 19 bp, Artificial Sequence
  A synthetic oligonucleotide
ggctttgtccagctccact

SEQ ID NO: 21 - DNA, 20 bp, Artificial Sequence
  A synthetic oligonucleotide
tgtttgaaatgtgcggagtg

SEQ ID NO: 22 - DNA, 20 bp, Artificial Sequence
  A synthetic oligonucleotide
gcatttgatcccgtacaacc

SEQ ID NO: 23 - DNA, 19 bp, Artificial Sequence
  A synthetic oligonucleotide
agctgtcttcccgtccatc

SEQ ID NO: 24 - DNA, 20 bp, Artificial Sequence
  A synthetic oligonucleotide
gctctgggcttcatcaccta

SEQ ID NO: 25 - DNA, 20 bp, Artificial Sequence
  A synthetic oligonucleotide
atgaggcccatcactgtagg

SEQ ID NO: 26 - DNA, 20 bp, Artificial Sequence
  A synthetic oligonucleotide
aaaattggagacgatgacgg

SEQ ID NO: 27 - DNA, 20 bp, Artificial Sequence
  A synthetic oligonucleotide
cccagctaagctcccataag

SEQ ID NO: 28 - DNA, 19 bp, Artificial Sequence
  A synthetic oligonucleotide
aacaggcagccaaatgtga

SEQ ID NO: 29 - DNA, 24 bp, Artificial Sequence
  A synthetic oligonucleotide
ctggtgatatagtggaaattgctg

SEQ ID NO: 30 - DNA, 20 bp, Artificial Sequence
  A synthetic oligonucleotide
ggtcagggacagggtcagta

SEQ ID NO: 31 - DNA, 20 bp, Artificial Sequence
  A synthetic oligonucleotide
ttgaggacacctgtggagtg

SEQ ID NO: 32 - DNA, 21 bp, Artificial Sequence
  A synthetic oligonucleotide
ttctcatactcctcgtcatcg

SEQ ID NO: 33 - DNA, 20 bp, Artificial Sequence
  A synthetic oligonucleotide
ccatgatcctgtaccagcaa

SEQ ID NO: 34 - DNA, 20 bp, Artificial Sequence
  A synthetic oligonucleotide
agagatggatgcttccgaga

SEQ ID NO: 35 - DNA, 20 bp, Artificial Sequence
  A synthetic oligonucleotide
ctaccgcatagtggagcaca

SEQ ID NO: 36 - DNA, 20 bp, Artificial Sequence
  A synthetic oligonucleotide
cgctcctccaggtagatgtc

SEQ ID NO: 37 - DNA, 20 bp, Artificial Sequence
  A synthetic oligonucleotide
gcgcattgagttcaagaaga

SEQ ID NO: 38 - DNA, 20 bp, Artificial Sequence
  A synthetic oligonucleotide
cttcatccatggccaattct

SEQ ID NO: 39 - DNA, 21 bp, Artificial Sequence
  A synthetic oligonucleotide
aagggcctgaatgaggagtag

SEQ ID NO: 40 - DNA, 20 bp, Artificial Sequence
  A synthetic oligonucleotide
tgcaaaggctccaggtctga

SEQ ID NO: 41 - DNA, 20 bp, Artificial Sequence
  A synthetic oligonucleotide
gtgtgcagtccctgttcaga

SEQ ID NO: 42 - DNA, 19 bp, Artificial Sequence
  A synthetic oligonucleotide
accctcaggctcaggttca

SEQ ID NO: 43 - DNA, 20 bp, Artificial Sequence
  A synthetic oligonucleotide
gcctctggagatcatcatgg

SEQ ID NO: 44 - DNA, 20 bp, Artificial Sequence
  A synthetic oligonucleotide
ctcggtagttggcagaggag

SEQ ID NO: 45 - DNA, 24 bp, Artificial Sequence
  A synthetic oligonucleotide
cttcactatcatggaccagaacag

SEQ ID NO: 46 - DNA, 24 bp, Artificial Sequence
  A synthetic oligonucleotide
acactttgaatgcgttgagaatgg

SEQ ID NO: 47 - DNA, 20 bp, Artificial Sequence
  A synthetic oligonucleotide
ctcacactcttcggggagaa

SEQ ID NO: 48 - DNA, 20 bp, Artificial Sequence
  A synthetic oligonucleotide
ctcttccttgttcaccaccc

SEQ ID NO: 49 - DNA, 20 bp, Artificial Sequence
  A synthetic oligonucleotide
cagttgaacagcagccagag

SEQ ID NO: 50 - DNA, 20 bp, Artificial Sequence
  A synthetic oligonucleotide
agctccagtcacccatcttg

SEQ ID NO: 51 - DNA, 22 bp, Artificial Sequence
  A synthetic oligonucleotide
acatcatgttttaccgcctgag

SEQ ID NO: 52 - DNA, 23 bp, Artificial Sequence
  A synthetic oligonucleotide
tttgtggttattgaactctggct

SEQ ID NO: 53 - DNA, 22 bp, Artificial Sequence
  A synthetic oligonucleotide
cctccagcagatgcaagaactc

SEQ ID NO: 54 - DNA, 23 bp, Artificial Sequence
  A synthetic oligonucleotide
cttcaaccactggtttttctgcc

SEQ ID NO: 55 - DNA, 20 bp, Artificial Sequence
  A synthetic oligonucleotide
tatgactcacttccaggggg

SEQ ID NO: 56 - DNA, 20 bp, Artificial Sequence
  A synthetic oligonucleotide
agaagaaagcaggatcgcct

SEQ ID NO: 57 - DNA, 20 bp, Artificial Sequence
  A synthetic oligonucleotide
ctcgccaactcagattcgat

SEQ ID NO: 58 - DNA, 20 bp, Artificial Sequence
  A synthetic oligonucleotide
agaagtgttgcacggctttg

SEQ ID NO: 59 - DNA, 20 bp, Artificial Sequence
  A synthetic oligonucleotide
acgctggtgctctatgcaag

SEQ ID NO: 60 - DNA, 21 bp, Artificial Sequence
  A synthetic oligonucleotide
tcagttgctgcccattcatca

SEQ ID NO: 61 - DNA, 19 bp, Artificial Sequence
  A synthetic oligonucleotide
cactgctttgggagccttc

SEQ ID NO: 62 - DNA, 21 bp, Artificial Sequence
  A synthetic oligonucleotide
ggggcagcgattcatttttct

SEQ ID NO: 63 - DNA, 22 bp, Artificial Sequence
  A synthetic oligonucleotide
gagtcagcttgctcctttatgg

SEQ ID NO: 64 - DNA, 23 bp, Artificial Sequence
  A synthetic oligonucleotide
agacacaagaggtagggaattga

SEQ ID NO: 65 - DNA, 21 bp, Artificial Sequence
  A synthetic oligonucleotide
caacaagggtccatcctacgg

SEQ ID NO: 66 - DNA, 19 bp, Artificial Sequence
  A synthetic oligonucleotide
atctgggcggcctacatca

SEQ ID NO: 67 - DNA, 19 bp, Artificial Sequence
  A synthetic oligonucleotide
aagctgcggctagaggtca

SEQ ID NO: 68 - DNA, 20 bp, Artificial Sequence
  A synthetic oligonucleotide
ccctccctttgatggctgag

SEQ ID NO: 69 - DNA, 22 bp, Artificial Sequence
  A synthetic oligonucleotide
tctgcacattttaaccgaggtc

SEQ ID NO: 70 - DNA, 23 bp, Artificial Sequence
  A synthetic oligonucleotide
gccagcttgttctttacttcagc

SEQ ID NO: 71 - DNA, 22 bp, Artificial Sequence
  A synthetic oligonucleotide
atgttacaggcgtgcaaaatgg

SEQ ID NO: 72 - DNA, 21 bp, Artificial Sequence
  A synthetic oligonucleotide
tgatcgctatggctttctcca

SEQ ID NO: 73 - DNA, 19 bp, Artificial Sequence
  A synthetic oligonucleotide
tggggacctcagaaccctt

SEQ ID NO: 74 - DNA, 19 bp, Artificial Sequence
  A synthetic oligonucleotide
ggctgcatttggggaagtg

SEQ ID NO: 75 - DNA, 19 bp, Artificial Sequence
  A synthetic oligonucleotide
gatggcactctggtcaccg

SEQ ID NO: 76 - DNA, 19 bp, Artificial Sequence
  A synthetic oligonucleotide
gccgctcggaaaaggacaa

SEQ ID NO: 77 - DNA, 21 bp, Artificial Sequence
  A synthetic oligonucleotide
ccactccgggacatagacttg

SEQ ID NO: 78 - DNA, 20 bp, Artificial Sequence
  A synthetic oligonucleotide
aaaagcgcaggtctggtgag

SEQ ID NO: 79 - DNA, 22 bp, Artificial Sequence
  A synthetic oligonucleotide
gagacatccccctatttctacc

SEQ ID NO: 80 - DNA, 19 bp, Artificial Sequence
  A synthetic oligonucleotide
gctcagtccgctcatagcc

SEQ ID NO: 81 - DNA, 21 bp, Artificial Sequence
  A synthetic oligonucleotide
aaggctcctgtatcccctcac

SEQ ID NO: 82 - DNA, 21 bp, Artificial Sequence
  A synthetic oligonucleotide
tgaccttcttcaggcgtctac

SEQ ID NO: 83 - DNA, 21 bp, Artificial Sequence
  A synthetic oligonucleotide
tgttcctcttaatcctgccca

SEQ ID NO: 84 - DNA, 21 bp, Artificial Sequence
  A synthetic oligonucleotide
ccaacctgcacaagttccctt

SEQ ID NO: 85 - DNA, 20 bp, Artificial Sequence
  A synthetic oligonucleotide
tcgctgatgcactgcctatg

SEQ ID NO: 86 - DNA, 21 bp, Artificial Sequence
  A synthetic oligonucleotide
gagaggtccacagagctgatt

SEQ ID NO: 87 - DNA, 19 bp, Artificial Sequence
  A synthetic oligonucleotide
ccagcctgagggcttactg

SEQ ID NO: 88 - DNA, 22 bp, Artificial Sequence
  A synthetic oligonucleotide
ctccattgactgtgacatctcg

SEQ ID NO: 89 - DNA, 19 bp, Artificial Sequence
  A synthetic oligonucleotide
agccgcaatgcaggtttct

SEQ ID NO: 90 - DNA, 22 bp, Artificial Sequence
  A synthetic oligonucleotide
ttgtgctcttgtctgtgtgaat

SEQ ID NO: 91 - DNA, 20 bp, Artificial Sequence
  A synthetic oligonucleotide
gagccggatctgaagaggga

SEQ ID NO: 92 - DNA, 20 bp, Artificial Sequence
  A synthetic oligonucleotide
gcttgacgtgtggcttgttc

SEQ ID NO: 93 - DNA, 20 bp, Artificial Sequence
  A synthetic oligonucleotide
cctgctacttcatcgacccc

SEQ ID NO: 94 - DNA, 21 bp, Artificial Sequence
  A synthetic oligonucleotide
agatgctgttgactcgaacct

SEQ ID NO: 95 - DNA, 20 bp, Artificial Sequence
  A synthetic oligonucleotide
ttctttgcagctccttcgtt

SEQ ID NO: 96 - DNA, 19 bp, Artificial Sequence
  A synthetic oligonucleotide
atggaggggaatacagccc

SEQ ID NO: 97 - DNA, 20 bp, Artificial Sequence
  A synthetic oligonucleotide
tctctcttcttttccggctg

SEQ ID NO: 98 - DNA, 20 bp, Artificial Sequence
  A synthetic oligonucleotide
ttcttgagggtttctggcac

SEQ ID NO: 99 - DNA, 19 bp, Artificial Sequence
  A synthetic oligonucleotide
cctgctggtggttagcgtg

SEQ ID NO: 100 - DNA, 21 bp, Artificial Sequence
  A synthetic oligonucleotide
tctgcctccgtctgtttggaa